(19) United States
(12) Patent Application Publication
(10) Pub. No.: US 2005/0256003 A1
(43) Pub. Date: Nov. 17, 2005

(54) Novel herbicides

(76) Inventors: Christoph Luthy, Basel (CH); Renaud Beauedegnies, Basel (CH); Andrew Edmunds, Basel (CH); Roger Graham Hall, Basel (CH); Sebastian Volker Wendeborn, Basel (CH)

Correspondence Address: SYNGENTA CROP PROTECTION , INC.;PATENT AND TRADEMARK DEPARTMENT, 410 SWING ROAD, GREENSBORO, NC, 27409, US

(21) Appl. No.: 10/517,964

(22) PCT Filed: Jun. 13, 2003
(86) PCT No.: PCT/EP03/06273

§ 371 Date: Dec. 14, 2004

(30) Foreign Application Priority Data
Jun. 14, 2002 (CH) 1029/02

(51) Int. Cl. A01N 25/00; A01N 43/48; C07D 43/02

(52) U.S. Cl. 504/251; 504/253; 546/268.4; 546/275.4

(57) ABSTRACT

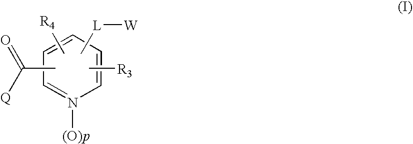

Compounds of formula (I) wherein the substituents are as defined in claim 1, and the agrochemically acceptable salts and all stereoisomeric and tautomeric forms of compounds of formula (I) are suitable for use as herbicides.

The present invention relates to novel, herbicidally active nicotinoyl derivatives, to processes for their preparation, to compositions comprising such compounds, and to their use in the control of weeds, especially in crops of useful plants, or in the inhibition of plant growth.

Nicotinoyl derivatives having herbicidal action are described, for example, in WO 00/15615, WO 00/39094 and WO 01/94339. Novel nicotinoyl derivatives having herbicidal and growth-inhibiting properties have now been found.

The present invention accordingly relates to compounds of formula I 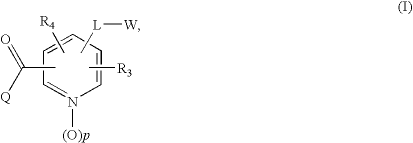
wherein
L is either a direct bond, an —O—, —S—, —S(O)—, —SO₂—, —N(R₅ₐ)—, —SO₂N(R_{5b})—, —N(R_{5b})SO₂—, —C(O)N(R_{5c})— or —N(R_{5c})C(O)— bridge, or a C₁-C₄alkylene, C₂-C₄alkenylene or C₂-C₄alkynylene chain which may be mono- or poly-substituted by R₅ and/or interrupted once or twice by an —O—, —S—, —S(O)—, —SO₂—, —N(R_{5d})—, —SO₂N(R₅ₑ)—, —N(R₅ₑ)SO₂—, —C(O)N(R_{5f})— and/or —N(R_{5f})C(O)— bridge, and when two such bridges are present those bridges are separated at least by one carbon atom, and W is bonded to L by way of a carbon atom or a —N(R₅ₑ)SO₂— or —N(R_{5f})C(O)— bridge when the bridge L is bonded to the nitrogen atom of W;
W is a 4- to 7-membered, saturated, partially saturated or unsaturated ring system U 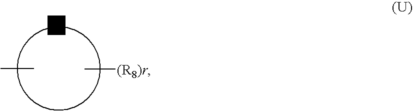
   which contains a ring element U₁, and may contain from one to four further ring nitrogen atoms, and/or two further ring oxygen atoms, and/or two further ring sulfur atoms and/or one or two further ring elements U₂, and the ring system U may be mono- or poly-substituted at a saturated or unsaturated ring carbon atom and/or at a ring nitrogen atom by a group R₈, and two substituents R₈ together are a further fused-on or spirocyclic 3- to 7-membered ring system which may be unsaturated, partially saturated or fully saturated and may in turn be substituted by one or more groups R₈ₐ and/or interrupted once or twice by a ring element —O—, —S—, —N(R_{8b})— and/or —C(═O)—; and
U₁ and U₂ are each independently of the other(s) —C(═O)—, —C(═S)—, —C(═NR₆)—, —(N═O)—, —S(═O)— or —SO₂—;
R₃ and R₄ are each independently of the other C₁-C₃alkyl, C₁-C₃haloalkyl, C₁-C₃alkoxy-C₁-C₃alkyl, hydrogen, hydroxy, mercapto, halogen, C₁-C₃alkoxy, C₁-C₃haloalkoxy, C₁-C₃alkoxy-C₁-C₃alkoxy, C₁-C₃alkylthio, C₁-C₃alkylsulfinyl, C₁-C₃alkylsulfonyl, C₁-C₃halo-alkylthio, C₁-C₃haloalkylsulfinyl, C₁-C₃haloalkylsulfonyl or C₁-C₃alkylsulfonyloxy;
R₅ is halogen, C₁-C₃alkyl, C₁-C₃alkoxy, C₁-C₃alkylthio, C₁-C₃alkylsulfinyl, C₁-C₃alkylsulfonyl, C₁-C₃alkoxy-C₁-C₃alkyl or C₁-C₃alkoxy-C₁-C₃alkoxy;
R₅ₐ, R_{5b} and R₅ₑ are independently hydrogen, C₁-C₆alkyl, C₃-C₆alkenyl, C₃-C₆alkynyl or C₁-C₃alkoxy-C₁-C₃alkyl;
R_{5d} is hydrogen, C₁-C₆alkyl, C₃-C₆alkenyl, C₃-C₆alkynyl, C₁-C₃alkoxy-C₁-C₃alkyl, benzyl, cyano, formyl, C₁-C₄alkylcarbonyl, C₁-C₄alkoxycarbonyl, C₁-C₄alkylsulfonyl or phenylsulfonyl, it being possible for the phenyl-containing groups to be substituted by R₇;
R_{5c} and R_{5f} are each independently of the other hydrogen or C₁-C₃alkyl;
R₆ is C₁-C₆alkyl, hydroxy, C₁-C₆alkoxy, cyano or nitro;
R₇ is halogen, C₁-C₃alkyl, C₁-C₃haloalkyl, hydroxy, C₁-C₃alkoxy, C₁-C₃haloalkoxy, cyano or nitro;
each R₈ independently is hydrogen, halogen, C₁-C₆alkyl, C₁-C₆haloalkyl, C₃-C₆cycloalkyl, C₂-C₆alkenyl, C₂-C₆alkynyl, hydroxy, C₁-C₆alkoxy, C₁-C₆haloalkoxy, C₃-C₆alkenyloxy, C₃-C₆alkynyloxy, C₁-C₃alkoxy-C₁-C₃alkoxy, mercapto, C₁-C₆alkylthio, C₁-C₆alkylsulfinyl, C₁-C₆alkylsulfonyl, C₁-C₆alkylsulfonyloxy, C₁-C₆haloalkylsulfonyloxy, C₃-C₆alkenylthio, C₃-C₆alkynylthio, amino, C₁-C₆alkylamino, di(C₁-C₆alkyl)amino, C₁-C₃alkoxy-C₁-C₃alkyl, formyl, C₁-C₄alkylcarbonyl, C₁-C₄alkoxycarbonyl, benzyloxycarbonyl, C₁-C₄alkylthiocarbonyl, carboxy, cyano, carbamoyl, phenyl, benzyl, heteroaryl or heterocyclyl, it being possible for the phenyl, benzyl, heteroaryl and heterocyclyl groups to be mono- or poly-substituted by R₇ₐ;
each R₇ₐ independently is halogen, C₁-C₃alkyl, C₁-C₃haloalkyl, hydroxy, C₁-C₃alkoxy, C₁-C₃haloalkoxy, cyano or nitro;
each R₇ₐ independently is halogen, C₁-C₆alkyl, C₁-C₆haloalkyl, C₃-C₆cycloalkyl, C₂-C₆alkenyl, C₂-C₆alkynyl, hydroxy, C₁-C₆alkoxy, C₁-C₆haloalkoxy, C₃-C₆alkenyloxy, C₃-C₆alkynyloxy, mercapto, C₁-C₆alkylthio, C₁-C₆alkylsulfinyl, C₁-C₆alkylsulfonyl, C₁-C₄alkylcarbonyl, C₁-C₄alkoxycarbonyl, cyano or nitro;
R_{8b} is hydrogen, C₁-C₃alkyl, C₃-C₆alkenyl, C₃-C₆alkynyl, C₁-C₃alkoxy-C₁-C₃alkyl or benzyl, it being possible for the phenyl group to be substituted by R_{7b};
R_{7b} is halogen, C₁-C₃alkyl, C₁-C₃haloalkyl, hydroxy, C₁-C₃alkoxy, C₁-C₃haloalkoxy, cyano or nitro;
p is 0 or 1;
r is 1, 2, 3, 4, 5 or 6;
with the provisos that
a) R₈ and R₈ₐ as halogen or hydrogenmercapto cannot be bonded to a nitrogen atom,
b) U₁ as —C(═O)— or —C(═S)— does not form a tautomeric form with a substituent R₈ as hydrogen when the radical W is bonded to the pyridyl group by way of a C₁-C₄alkylene, C₂-C₄alkenylene or C₂-C₄alkynylene chain L that is interrupted by —O—, —S—, —S(O)—, —SO₂—, —N(R_{5d})—, —SO₂N(R₅ₑ)— or —N(R₅ₑ)SO₂—,
c) U₁ as —C(═S)— does not form a tautomeric form with a substituent R₈ as hydrogen when the radical W is bonded to the pyridyl group by way of a —CH═CH— or —C≡C— bridge L or by way of a C₁-C₄alkylene chain L that is interrupted by —O—, —S—, —S(O)—, —SO₂— or —N(C₁-C₄alkyl)—,
d) U₁ as —C(═S)— or —C(═NR₆)— wherein R₆ is C₁-C₆alkyl or C₁-C₆alkoxy does not form a tautomeric form with a substituent R₈ as hydrogen when the radical W is bonded to the pyridyl group directly or by way of a C₁-C₄alkylene chain L; either
Q is a group Q₁ 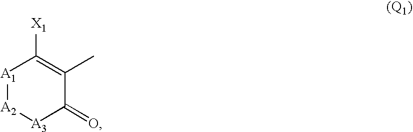
   wherein
A₁ is C(R₁₁R₁₂) or NR₁₃;
A₂ is C(R₁₄R₁₅)ₘ, C(O), oxygen, NR₁₆ or S(O)_{q};
A₃ is C(R₁₇R₁₈) or NR₁₉;
   with the proviso that A₂ is other than S(O)_{q} when A₁ is NR₁₃ and/or A₃ is NR₁₉;
X₁ is hydroxy, O⁻M⁺, wherein M⁺ is a metal cation or an ammonium cation; halogen or S(O)ₙR₉,
   wherein
m is 1 or 2;
q, n and k are each independently of the others 0, 1 or 2;
R₉ is C₁-C₁₂alkyl, C₂-C₁₂alkenyl, C₂-C₁₂alkynyl, C₃-C₁₂allenyl, C₃-C₁₂cycloalkyl, C₅-C₁₂cyclo-alkenyl, R₁₀-C₁-C₁₂alkylene or R₁₀-C₂-C₁₂alkenylene, wherein the alkylene or alkenylene chain may be interrupted by —O—, —S(O)ₖ— and/or —C(O)— and/or mono- to penta-substituted by R₂₀; or phenyl, which may be mono- to penta-substituted by R_{7c};
R_{7c} is halogen, C₁-C₃alkyl, C₁-C₃haloalkyl, hydroxy, C₁-C₃alkoxy, C₁-C₃haloalkoxy, cyano or nitro;
R₁₀ is halogen, cyano, rhodano, hydroxy, C₁-C₆alkoxy, C₂-C₆alkenyloxy, C₂-C₆alkynyloxy, C₁-C₆alkylthio, C₁-C₆alkylsulfinyl, C₁-C₆alkylsulfonyl, C₂-C₆alkenylthio, C₂-C₆alkynylthio, C₁-C₆alkylsulfonyloxy, phenylsulfonyloxy, C₁-C₆alkylcarbonyloxy, benzoyloxy, C₁-C₄alkoxy-carbonyloxy, C₁-C₆alkylcarbonyl, C₁-C₄alkoxycarbonyl, benzoyl, aminocarbonyl, C₁-C₄alkyl-aminocarbonyl, C₃-C₆cycloalkyl, phenyl, phenoxy, phenylthio, phenylsulfinyl or phenyl-sulfonyl; it being possible for the phenyl-containing groups in turn to be substituted by R_{7d};
R_{7d} is halogen, C₁-C₃alkyl, C₁-C₃haloalkyl, hydroxy, C₁-C₃alkoxy, C₁-C₃haloalkoxy, cyano or nitro;
R₂₀ is hydroxy, halogen, C₁-C₆alkyl, C₁-C₆alkoxy, C₁-C₆alkylthio, C₁-C₆alkylsulfinyl, C₁-C₆alkylsulfonyl, cyano, carbamoyl, carboxy, C₁-C₄alkoxycarbonyl or phenyl; it being possible for phenyl to be substituted by R₇ₑ;
R₇ₑ is halogen, C₁-C₃alkyl, C₁-C₃haloalkyl, hydroxy, C₁-C₃alkoxy, C₁-C₃haloalkoxy, cyano or nitro; R₁₁ and R₁₇ are each independently of the other hydrogen, C₁-C₄alkyl, C₂-C₄alkenyl, C₂-C₄alkynyl, C₁-C₄alkylthio, C₁-C₄alkylsulfinyl, C₁-C₄alkylsulfonyl, C₁-C₄alkoxycarbonyl, hydroxy, C₁-C₄alkoxy, C₃-C₄alkenyloxy, C₃-C₄alkynyloxy, hydroxy-C₁-C₄alkyl, C₁-C₄alkyl-sulfonyloxy-C₁-C₄alkyl, halogen, cyano or nitro;
or, when A₂ is C(R₁₄R₁₅)ₘ, R₁₇ together with R₁₁ forms a direct bond or a C₁-C₃alkylene bridge;
R₁₂ and R₁₈ are each independently of the other hydrogen, C₁-C₄alkyl or C₁-C₄alkylthio, C₁-C₄alkylsulfinyl or C₁-C₄alkylsulfonyl;
or R₁₂ together with R₁₁, and/or R₁₈ together with R₁₇ form a C₂-C₅alkylene chain which may be interrupted by —O—, —C(O)—, —O— and —C(O)— or —S(O)ₜ—;
R₁₃ and R₁₉ are each independently of the other hydrogen, C₁-C₄alkyl, C₁-C₄haloalkyl, C₃-C₄alkenyl, C₃-C₄alkynyl or C₁-C₄alkoxy;
R₁₄ is hydrogen, hydroxy, C₁-C₄alkyl, C₁-C₄haloalkyl, C₁-C₃hydroxyalkyl, C₁-C₄alkoxy-C₁-C₃-alkyl, C₁-C₄alkylthio-C₁-C₃alkyl, C₁-C₄alkylcarbonyloxy-C₁-C₃alkyl, C₁-C₄alkylsulfonyloxy-C₁-C₃alkyl, tosyloxy-C₁-C₃alkyl, di(C₁-C₄alkoxy)-C₁-C₃alkyl, C₁-C₄alkoxycarbonyl, C₃-C₅-oxacycloalkyl, C₃-C₅thiacycloalkyl, C₃-C₄dioxacycloalkyl, C₃-C₄dithiacycloalkyl, C₃-C₄oxa-thiacycloalkyl, formyl, C₁-C₄alkoxyiminomethyl, carbamoyl, C₁-C₄alkylaminocarbonyl or di-(C₁-C₄alkyl)aminocarbonyl;
or R₁₄ together with R₁₁, R₁₂, R₁₃, R₁₅, R₁₇, R₁₈ or R₁₉ or, when m is 2, also together with R₁₄ forms a direct bond or a C₁-C₄alkylene bridge;
R₁₅ is hydrogen, C₁-C₃alkyl or C₁-C₃haloalkyl;
R₁₆ is hydrogen, C₁-C₃alkyl, C₀-C₃haloalkyl, C₁-C₄alkoxycarbonyl, C₁-C₄alkylcarbonyl or N,N-di(C₁-C₄alkyl)aminocarbonyl; or
Q is a group Q₂ 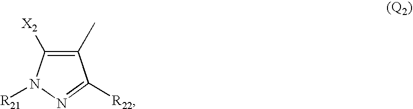
   wherein
R₂₁ and R₂₂ are hydrogen or C₁-C₄alkyl;
X₂ is hydroxy, O⁻M⁺, wherein M⁺ is an alkali metal cation or ammonium cation; halogen, C₁-C₁₂alkylsulfonyloxy, C₁-C₁₂alkylthio, C₁-C₁₂alkylsulfinyl, C₁-C₁₂alkylsulfonyl, C₁-C₁₂halo-alkylthio, C₁-C₁₂haloalkylsulfinyl, C₁-C₁₂haloalkylsulfonyl, C₁-C₆alkoxy-C₁-C₆alkylthio, C₁-C₆-alkoxy-C₁-C₆alkylsulfinyl, C₁-C₆alkoxy-C₁-C₆alkylsulfonyl, C₃-C₁₂alkenylthio, C₃-C₁₂alkenyl-sulfinyl, C₃-C₁₂alkenylsulfonyl, C₃-C₁₂alkynylthio, C₃-C₁₂alkynylsulfinyl, C₃-C₁₂alkynylsulfonyl, C₁-C₄alkoxycarbonyl-C₁-C₄alkylthio, C₁-C₄alkoxycarbonyl-C₁-C₄alkylsulfinyl, C₁-C₄alkoxy-carbonyl-C₁-C₄alkylsulfonyl, benzyloxy or phenylcarbonylmethoxy; it being possible for the phenyl-containing groups to be substituted by R_{7f};
R_{7f} is halogen, C₁-C₃alkyl, C₁-C₃haloalkyl, hydroxy, C₁-C₃alkoxy, C₁-C₃haloalkoxy, cyano or nitro; or
Q is a group Q₃ 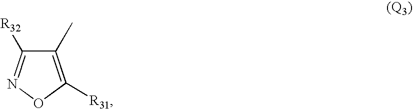
   wherein
R₃₁ is C₁-C₆alkyl, C₁-C₆haloalkyl, C₃-C₆cycloalkyl or halo-substituted C₃-C₆cycloalkyl;
R₃₂ is hydrogen, C₁-C₄alkoxycarbonyl, carboxy or a group S(O)₅R₃₃;
R₃₃ is C₁-C₆alkyl or C₁-C₃alkylene, which may be substituted by halogen, C₁-C₃alkoxy, C₂-C₃alkenyl or by C₂-C₃alkynyl; and
s is 0, 1 or 2; or
Q is a group Q₄ 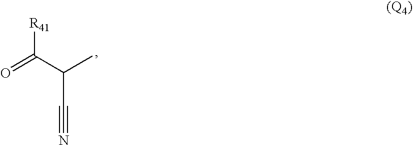
   wherein
R₄₁ is C₁-C₆alkyl, C₁-C₆haloalkyl, C₃-C₆cycloalkyl or halo-substituted C₃-C₆cycloalkyl; and to the agrochemically acceptable salts and to all stereoisomers and tautomers of compounds of formula I.

The alkyl groups appearing in the substituent definitions may be straight-chain or branched and are, for example, methyl, ethyl, n-propyl, isopropyl, n-butyl, sec-butyl, isobutyl, tert-butyl, pentyl, hexyl, heptyl, octyl, nonyl, decyl, undecyl and dodecyl and the branched isomers thereof. Alkoxy, alkenyl and alkynyl radicals are derived from the mentioned alkyl radicals. The alkenyl and alkynyl groups may be mono- or poly-unsaturated, C₂-C₁₁alkyl chains having one or more double or triple bonds also being included. Alkenyl is, for example, vinyl, allyl, isobuten-3-yl, CH₂═CH—CH₂—CH═CH₂—, CH₂═CH—CH₂—CH₂—CH═CH₂— or CH₃—CH═CH—CH₂CH═CH—. A preferred alkynyl is, for example, propargyl, and CH₂═C═CH₂— is a preferred allenyl.

An alkylene chain may be substituted by one or more C₁-C₃alkyl groups, especially by methyl groups; such alkylene chains and alkylene groups are preferably unsubstituted. The same applies to all groups containing C₃-C₆cycloalkyl, C₃-C₄oxacycloalkyl, C₃-C₅thiacycloalkyl, C₃-C₄dioxacycloalkyl, C₃-C₄dithiacycloalkyl or C₃-C₄oxaathiacycloalkyl.

An alkylene chain uninterrupted or interrupted by oxygen, S(O)ₖ, —S(O)ₗ, —NR₅— or by carbonyl and especially a C₁-C₄alkylene chain L which can be unsubstituted or substituted one or more times (up to five times) by R₅ and/or uninterrupted or interrupted once or twice by —O—, —S(O)ₗ—, —N(R_{5d})—, —SO₂N(R₅ₑ)—, —N(R₅ₑ)SO₂—, —C(O)N(R_{5f})— or —N(R_{5f})C(O)—, the latter being separated at least by one carbon atom, and W is bonded to L by way of a carbon atom or a —N(R₅ₑ)SO₂— or —N(R_{5f})C(O)— bridge when the bridge L is bonded to the nitrogen atom of W; is to be understood as being, for example, a chain —CH₂—, —CH₂CH₂—, —CH₂CH₂CH₂—, —CH₂CH₂CH₂CH₂—, —CH(CH₃)—, —CH₂CH(CH₃)—, —CH₂CH(CH₃)CH₂—, —CH₂CH(Cl)CH₂—, —CH₂CH(OCH₃)CH₂—, —CH₂O—, —OCH₂—, —CH₂OCH₂—, —OCH₂CH₂—, —OCH₂CH₂CH₂—, —CH₂OCH₂CH₂—, —CH₂OCH(CH₃)CH₂—, —SCH₂—, —SCH₂CH₂—, —SCH₂CH₂CH₂—, —CH₂S—, —CH₂SCH₂—, —CH₂S(O)CH₂—, —CH₂SO₂CH₂—, —CH₂SCH₂CH₂—, —CH₂S(O)CH₂CH₂—, —CH₂SO₂CH₂CH₂—, —CH₂SO₂NH—, —CH₂N(CH₃)SO₂CH₂CH₂—, —N(SO₂Me)CH₂CH₂—, —CH₂C(O)NH— or —CH₂NHC(O)CH₂—. The definition R₁₀—C₁-C₁₂alkylene which may be interrupted by oxygen or by —S(O)ₙ— denotes, for example, CH₃OCH₂CH₂O—, phenoxy, phenoxymethyl, benzyloxy, benzylthio or benzyloxymethyl.

A C₂-C₄alkenylene chain which can be uninterrupted or interrupted by oxygen is accordingly to be understood as being, for example, —CH═CH—CH₂—, —CH═CH—CH₂CH₂— or —CH═CHCH₂OCH₂—, and a C₂-C₄alkynylene chain which can be uninterrupted or interrupted by oxygen is to be understood as being, for example, —C≡C—, —C≡CCH₂—, —C≡CCH₂O—, —C≡CCH₂OCH₂— or —OC≡CCH₂—.

An alkylene chain which can be mono- or poly-substituted by R₅ in C₁-C₄alkylene or by R₂₀ in R₁₀—C₁-C₁₂alkylene can be substituted, for example, up to five times. Two such substituents as C₁-C₃alkyl can together also form a 3- to 8-membered ring, the groups in question being located at the same carbon atom or at adjacent atoms.

W as a 4- to 7-membered, saturated, partially saturated or unsaturated ring system U 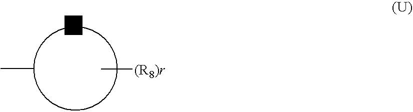
is to be understood as being especially a heterocyclic ring system U which contains a ring element U₁ and which may contain from one to four further ring nitrogen atoms, and/or one or two further ring oxygen atoms, and/or one or two further ring sulfur atoms and/or one or two further ring elements U₂, and which may be substituted one or more times (e.g. up to six times) at a saturated or unsaturated ring carbon atom and/or at a ring nitrogen atom by a group R₈, and in which two radicals R₈ together may be a further fused-on or spirocyclic 3- to 7-membered ring system, which may likewise be unsaturated, partially saturated or fully saturated and may itself be substituted by one or more groups R₈ₐ; and wherein U₁ and U₂ are each independently of the other —C(═O)—, —C(═S)—, —C(═NR₆)—, —(N═O)—, —S(═O)— or —SO₂—. Such ring systems U are, for example, 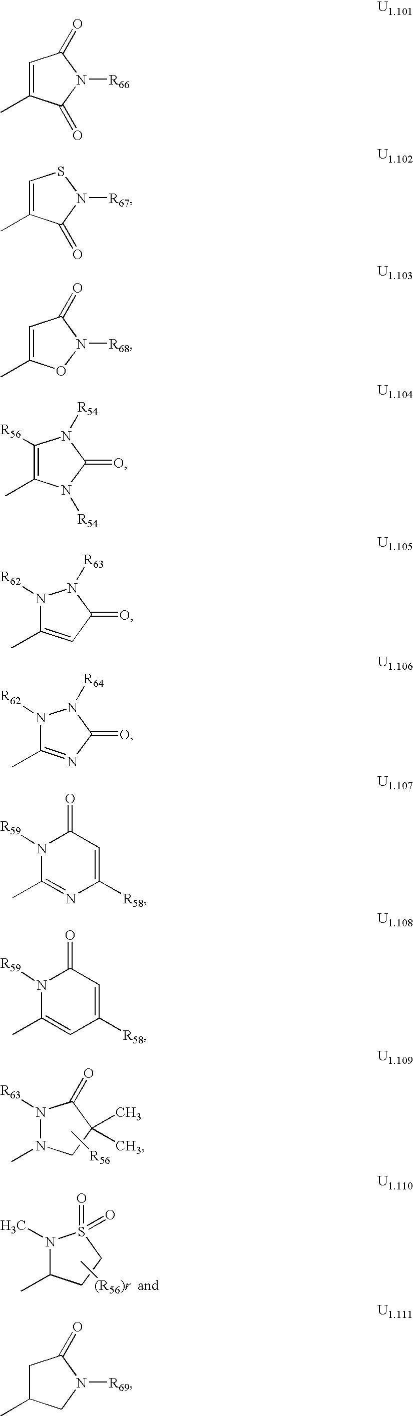
wherein R₅₄, R₅₆, R₅₈, R₅₉, R₆₂, R₆₃, R₆₆, R₆₇, R₆₈ and R₆₉ as sub-groups of selected substituents R₈ have the definitions and preferred meanings indicated hereinbelow.

Preferably W as a 4- to 7-membered, saturated, partially saturated or unsaturated ring system U is a heterocyclic group U₀ 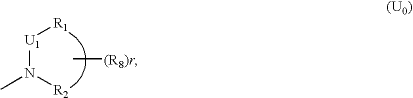
wherein R₁ together with R₂, by way of the nitrogen atom and the ring element U₁, forms the corresponding ring system U, which may additionally contain up to 3 nitrogen atoms, a further oxygen atom, a further sulfur atom or a further group U₂ and which may additionally be substituted one or more times (for example up to six times) at a saturated or unsaturated ring carbon atom and/or at a ring nitrogen atom by a group R₈, and in which two substituents R₈ together may be a further fused-on or spirocyclic 3- to 7-membered ring system, which may likewise be unsaturated, partially saturated or unsaturated and may itself be substituted by one or more groups R₈ₐ. W is especially a heterocycle selected from the groups 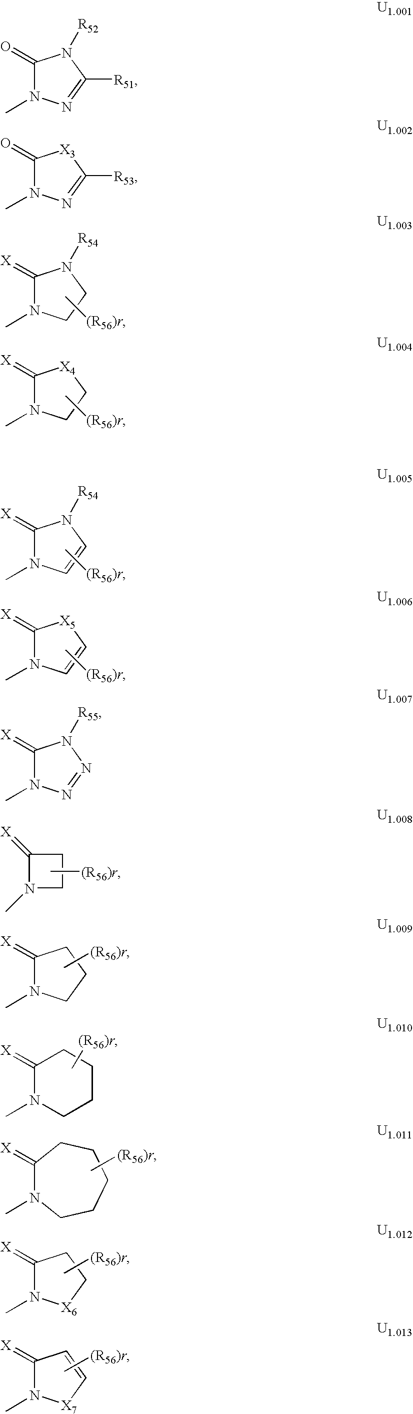 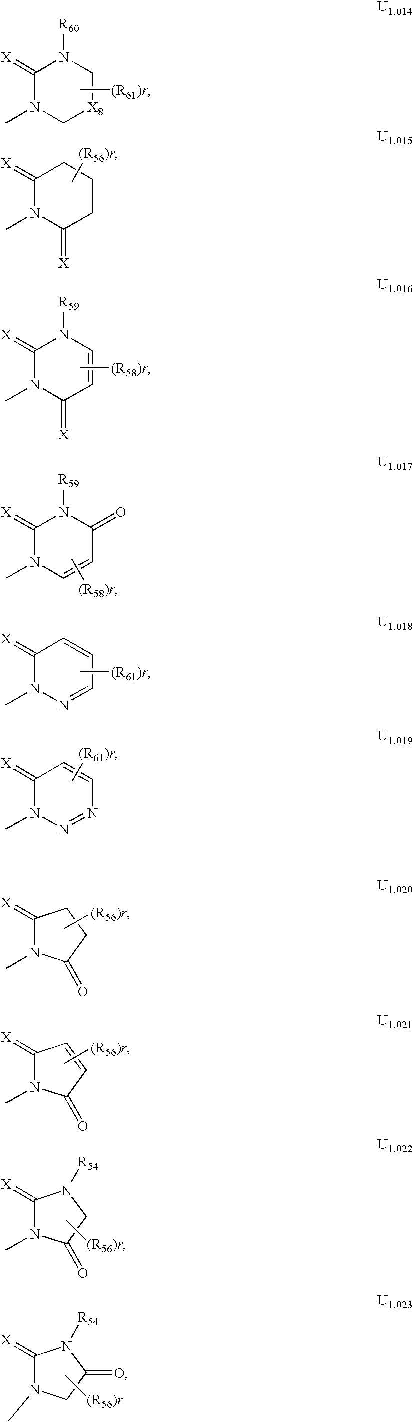 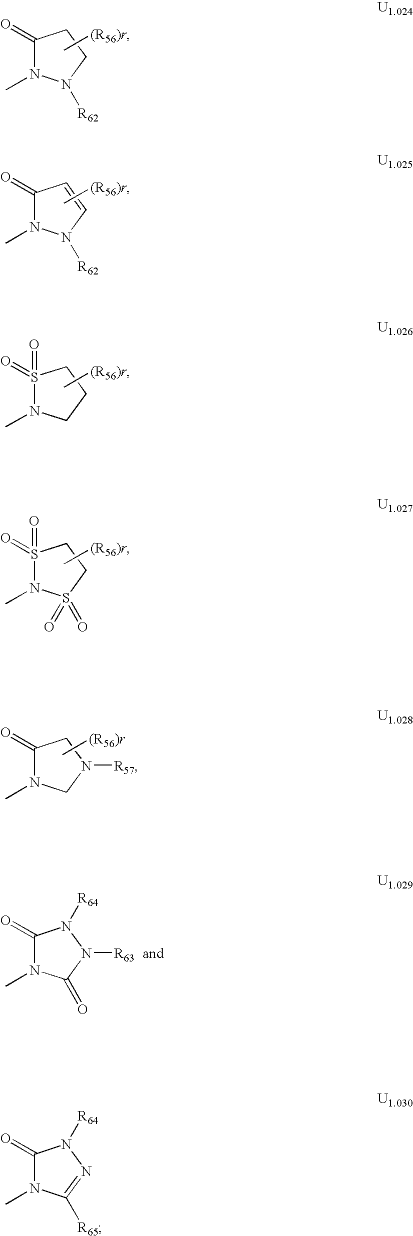
wherein R₅₁, R₅₃, R₅₆, R₆₅ are each independently of the others hydrogen, halogen, C₁-C₆-alkyl, C₁-C₆haloalkyl, C₃-C₆cycloalkyl, C₃-C₆alkenyl, C₃-C₆alkynyl, C₁-C₃alkoxy-C₁-C₃alkyl, C₁-C₆alkoxy, C₃-C₆alkenyloxy, C₃-C₆alkynyloxy, C₁-C₆alkylthio, C₁-C₆alkylsulfinyl, C₁-C₆alkyl-sulfonyl, C₃-C₆alkenylthio or C₃-C₆alkynylthio; R₅₂ is hydrogen, C₁-C₆alkyl, C₁-C₆haloalkyl, C₃-C₆cycloalkyl, C₃-C₆alkenyl, C₃-C₆alkynyl, C₁-C₆alkoxy, amino, or phenyl which may in turn be substituted by R₇₀; R₅₄, R₅₅, R₆₀ are hydrogen, C₁-C₆alkyl, C₁-C₆haloalkyl, C₃-C₆alkenyl, C₃-C₆alkynyl or C₃-C₆cycloalkyl; R₅₇, R₆₃, R₆₆, R₆₇, R₆₈, R₆₉ are C₁-C₆alkyl, or phenyl which may in turn be substituted by R₇₀; R₆₄ is C₁-C₆alkyl, C₁-C₆haloalkyl, C₃-C₆cycloalkyl, C₃-C₆-alkenyl, C₃-C₆alkynyl, or phenyl which may in turn be substituted by R₇₀; R₅₈, R₆, are hydrogen, halogen, C₁-C₆alkyl or C₁-C₆haloalkyl; R₅₉ is C₁-C₆alkyl, C₁-C₆haloalkyl, C₁-C₃-alkoxy-C₁-C₃alkyl, C₃-C₆alkenyl or C₃-C₆alkynyl; R₆₂ is hydrogen, C₁-C₆alkyl, C₁-C₄alkoxy-carbonyl or C₁-C₄alkylthiocarbonyl; or R₅₁ together with R₅₂, or R₅₄ together with an adjacent group R₅₆, or R₅₈ together with an adjacent group R₅₉, or R₆₀ together with an adjacent group R₆₁, or, when r is 2, two adjacent groups R₅₆ or two adjacent groups R₆₁ together may form a saturated or unsaturated C₁-C₅alkylene or C₃-C₄alkenylene bridge which may in turn be substituted by a group R₇₀ or interrupted by oxygen, sulfur or nitrogen; each R₇₀ independently is halogen, C₁-C₃alkyl, C₁-C₃haloalkyl, hydroxy, C₁-C₃alkoxy, C₁-C₃haloalkoxy, cyano or nitro; X is oxygen, sulfur or NR₆; X₃, X₄ and X₅ are oxygen or sulfur; X₆ and X₇ are oxygen or S, S(O), SO₂; and X₈ is CH₂, oxygen, S, S(O), SO₂ or NR₇₁, wherein R₇₁ is hydrogen or C₁-C₆alkyl.

Two substituents R₈ as hydroxy may be a further carbonyl group when they are located at the same carbon atom, and two substituents R₅ that together form a further 3- to 7-membered ring system can be located at the same carbon atom to form a spiro ring or at two adjacent carbon and/or nitrogen atoms to form a fused ring system, such as, for example, in the case of the groups: 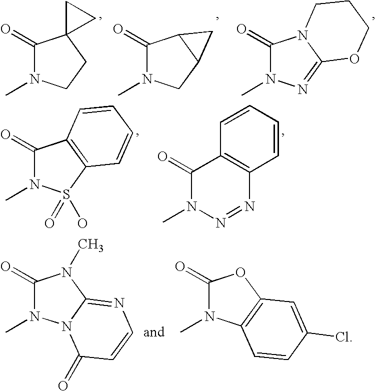
The provisos that U₁ as either —C(═O)— or —C(═S)— or —C(═NR_{5d})— does not form a tautomeric form with a substituent R₈ as hydrogen are to be understood as meaning especially that an enol form is not formed under physiological conditions in a pH range of from about 2 to about 11. Accordingly, the present invention likewise relates, for example, to compounds of formulae

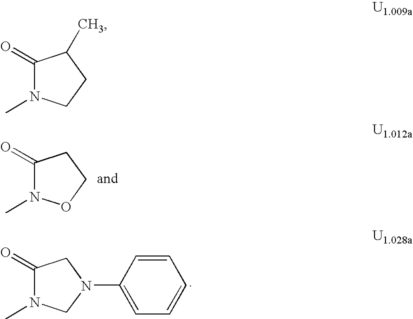

Halogen is generally fluorine, chlorine, bromine or iodine, preferably fluorine or chlorine. The same is true of halogen in conjunction with other meanings, such as haloalkyl, haloalkoxy or halophenyl.

Haloalkyl groups having a chain length of from 1 to 6 carbon atoms are, for example, fluoro-methyl, difluoromethyl, trifluoromethyl, chloromethyl, dichloromethyl, trichloromethyl, 2,2,2-trifluoroethyl, 1-fluoroethyl, 2-fluoroethyl, 2-chloroethyl, 2-fluoroprop-2-yl, pentafluoroethyl, 1,1-difluoro-2,2,2-trichloroethyl, 2,2,3,3-tetrafluoroethyl and 2,2,2-trichloroethyl, pentafluoro-ethyl, heptafluoro-n-propyl, perfluoro-n-hexyl. Preferred haloalkyl groups in the definitions R to Rₓ, and particularly the group R₃, are fluoromethyl, difluoromethyl, difluorochloromethyl, trifluoromethyl and pentafluoroethyl.

As haloalkenyl there come into consideration alkenyl groups mono- or poly-substituted by halogen, halogen being fluorine, chlorine, bromine or iodine, and especially fluorine or chlorine, for example 1-chlorovinyl, 2-chlorovinyl, 2,2-difluoro-vinyl, 2,2-difluoro-prop-1-en-2-yl, 2,2-dichloro-vinyl, 3-fluoroprop-1-enyl, chloroprop-1-en-1-yl, 3-bromoprop-1-en-1-yl, 3-iodoprop-1-en-1-yl, 2,3,3-trifluoroprop-2-en-1-yl, 2,3,3-trichloroprop-2-en-1-yl and 4,4,4-trifluoro-but-2-en-1-yl.

As haloalkynyl there come into consideration, for example, alkynyl groups mono- or poly-substituted by halogen, halogen being bromine, iodine and especially fluorine or chlorine, for example 3-fluoropropynyl, 3-chloropropynyl, 3-bromopropynyl, 3,3,3-trifluoropropynyl and 4,4,4trifluoro-but-2-yn-1-yl.

A C₃-C₆cycloalkyl group may likewise be mono- or poly-substituted by halogen, for example 2,2-dichlorocyclopropyl, 2,2-dibromocyclopropyl, 2,2,3,3-tetrafluorocyclobutyl or 2,2-difluoro-3,3-dichlorocyclobutyl.

Alkoxy groups preferably have a chain length of from 1 to 6 carbon atoms. Alkoxy is, for example, methoxy, ethoxy, propoxy, isopropoxy, n-butoxy, isobutoxy, sec-butoxy or tert-butoxy or a pentyloxy or hexyloxy isomer; preferably methoxy or ethoxy.

Haloalkoxy groups preferably have a chain length of from 1 to 6 carbon atoms, e.g. fluoro-methoxy, difluoromethoxy, trifluoromethoxy, 2,2,2-trifluoroethoxy, 1,1,2,2-tetrafluoroethoxy, 1-fluoroethoxy, 2-fluoroethoxy, 2-chloroethoxy, 2,2-difluoroethoxy and 2,2,2-trichloroethoxy; preferably fluoromethoxy, difluoromethoxy, 2-chloroethoxy and trifluoromethoxy.

Alkylthio groups preferably have a chain length of from 1 to 8 carbon atoms.

Alkylthio is, for example, methylthio, ethylthio, propylthio, isopropylthio, n-butylthio, isobutyl-thio, sec-butylthio or tert-butylthio, preferably methylthio or ethylthio. Alkylsulfinyl is, for example, methylsulfinyl, ethylsulfinyl, propylsulfinyl, isopropylsulfinyl, n-butylsulfinyl, isobutyl-sulfinyl, sec-butylsulfinyl, tert-butylsulfinyl; preferably methylsulfinyl or ethylsulfinyl.

Alkylsulfonyl is, for example, methylsulfonyl, ethylsulfonyl, propylsulfonyl, isopropylsulfonyl, n-butylsulfonyl, isobutylsulfonyl, sec-butylsulfonyl or tert-butylsulfonyl; preferably methyl-sulfonyl or ethylsulfonyl.

Alkylamino is, for example, methylamino, ethylamino, n-propylamino, isopropylamino or a butylamine isomer. Dialkylamino is, for example, dimethylamino, methylethylamino, diethyl-amino, n-propylmethylamino, dibutylamino or diisopropylamino. Alkylamino groups having a chain length of from 1 to 4 carbon atoms are preferred.

Alkoxyalkyl groups preferably have from 2 to 6 carbon atoms. Alkoxyalkyl is, for example, methoxymethyl, methoxyethyl, ethoxymethyl, ethoxyethyl, n-propoxymethyl, n-propoxyethyl, isopropoxymethyl or isopropoxyethyl. Alkoxy-alkoxyalkyl groups preferably have from 3 to 8 carbon atoms, e.g. methoxymethoxymethyl, methoxyethoxymethyl, ethoxymethoxymethyl, ethoxyethoxymethyl. Di(C₁-C₄alkoxy)-C₁-C₄alkyl is to be understood as being, for example, dimethoxymethyl or diethoxymethyl.

Alkylthioalkyl groups preferably have from 2 to 6 carbon atoms. Alkylthioalkyl is, for example, methylthiomethyl, methylthioethyl, ethylthiomethyl, ethylthioethyl, n-propylthiomethyl, n-propylthioethyl, isopropylthiomethyl, isopropylthioethyl, butylthiomethyl, butylthioethyl or butylthiobutyl.

Alkylcarbonyl is preferably acetyl or propionyl. Alkoxycarbonyl is, for example, methoxy-carbonyl, ethoxycarbonyl, propoxycarbonyl, isopropoxycarbonyl, n-butoxycarbonyl, iso-butoxycarbonyl, sec-butoxycarbonyl or tert-butoxycarbonyl; preferably methoxycarbonyl, ethoxycarbonyl or tert-butoxycarbonyl.

Phenyl, including as part of a substituent such as phenoxy, benzyl, benzyloxy, benzoyl, phenylthio, phenylalkyl, phenoxyalkyl or tosyl, can be in mono- or poly-substituted form. The substituents can in that case be as desired, preferably with a substituent having a meaning of R₇ in the ortho-, meta- and/or para-position.

Heteroaryl is to be understood as being a 5- or 6-membered group containing both nitrogen and oxygen and/or sulfur, for example furyl, thienyl, pyrrolyl, pyrazolyl, imidazolyl, triazolyl, oxazolyl, thiazolyl, pyridyl, pyrimidinyl, triazinyl, pyrrolyl, pyrazolyl, triazolyl, tetrazolyl, oxadiazolyl, thiadiazolyl, 4,5-dihydro-isoxazole, 2-pyranyl, 1,3-dioxol-2-yl, oxiranyl, 3-oxetanyl, tetrahydrofuranyl, tetrahydropyranyl or one of the groups U₁ defined above.

Heterocyclyl is to be understood as being a ring system containing, in addition to carbon atoms, at least one hetero atom, such as nitrogen, oxygen and/or sulfur. It can be saturated or unsaturated. Heterocyclyl ring systems in the context of the present invention can also be substituted. Suitable substituents are, for example, C₁-C₄alkyl, C₁-C₄haloalkyl, C₁-C₄alkoxy, cyano, nitro, C₁-C₄alkylsulfonyl, C₁-C₄alkylsulfinyl, C₁-C₄alkylthio and C₃-C₆cycloalkyl.

The present invention relates also to the salts which the compounds of formula I and especially the compounds of formula Ia are able to form with amines, alkali metal and alkaline earth metal bases or quaternary ammonium bases. Among the alkali metal and alkaline earth metal bases as salt formers, special mention should be made of the hydroxides of lithium, sodium, potassium, magnesium and calcium, but especially the hydroxides of sodium and potassium. Examples of amines suitable for ammonium salt formation include ammonia as well as primary, secondary and tertiary C₁-C₁₋₈alkylamines, C₁-C₄hydroxyalkyl-amines and C₂-C₄alkoxyalkylamines, for example methylamine, ethylamine, n-propylamine, isopropylamine, the four butylamine isomers, n-amylamine, isoamylamine, hexylamine, heptylamine, octylamine, nonylamine, decylamine, pentadecylamine, hexadecylamine, heptadecylamine, octadecylamine, methylethylamine, methylisopropylamine, methylhexyl-amine, methylnonylamine, methylpentadecylamine, methyloctadecylamine, ethylbutylamine, ethylheptylamine, ethyloctylamine, hexylheptylamine, hexyloctylamine, dimethylamine, diethylamine, di-n-propylamine, diisopropylamine, di-n-butylamine, di-n-amylamine, diiso-amylamine, dihexylamine, diheptylamine, dioctylamine, ethanolamine, n-propanolamine, iso-propanolamine, N,N-diethanolamine, N-ethylpropanolamine, N-butylethanolamine, allyl-amine, n-butenyl-2-amine, n-pentenyl-2-amine, 2,3-dimethylbutenyl-2-amine, dibutenyl-2-amine, n-hexenyl-2-amine, propylenediamine, trimethylamine, triethylamine, tri-n-propyl-amine, triisopropylamine, tri-n-butylamine, triisobutylamine, tri-sec-butylamine, tri-n-amyl-amine, methoxyethylamine and ethoxyethylamine; heterocyclic amines, for example pyridine, quinoline, isoquinoline, morpholine, piperidine, pyrrolidine, indoline, quinuclidine and azepine; primary arylamines, for example anilines, methoxyanilines, ethoxyanilines, o-, m- and p-toluidines, phenylenediamines, naphthylamines and o-, m- and p-chloroanilines; but especially triethylamine, isopropylamine and diisopropylamine. Quaternary ammonium bases suitable for salt formation are, for example, [N(Rₐ R_{b} R_{c} R_{d})]⁺OH⁻ wherein Rₐ, R_{b}, R_{c} and R_{d} are each independently of the others C₁-C₄alkyl. Further suitable tetraalkylammonium bases with other anions can be obtained, for example, by anion exchange reactions. M⁺ is preferably an ammonium salt, especially NH₄⁺, or an alkali metal, especially potassium or sodium.

Depending upon the preparation process, the compounds of formula I may be obtained in various tautomeric forms, such as, for example, in Form A shown below or in Form B or in Form C, preference being given to Form A, as shown by way of example for compounds of formula IA wherein Q is a group Q₁ and the group -L-W is in the 2-position. 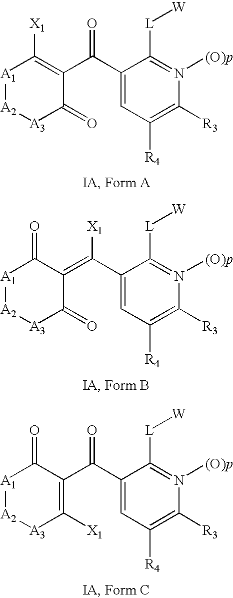
When X₁ is hydroxy, the structure of formula I can also be represented by the tautomeric Form D 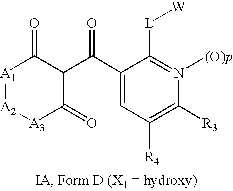
as shown likewise by way of the example of compounds of formula IA wherein Q is a group Q₁ and the group -L-W is in the 2-position. Compounds of formula I wherein Q is a group Q₂ or a group Q₄can accordingly be present in the tautomeric forms A, B, C or D. When a C═N or C═C double bond is present in compounds of formula I, the compounds of formula I, when asymmetric, may be in the E form or the Z form. When a further asymmetric centre is present, for example an asymmetric carbon atom, chiral R or S forms may occur. The present invention therefore relates also to all such stereoisomeric and tautomeric forms of the compound of formula I.

Of the compounds of formula I, the formulae IA, IB, IC, ID, IE, IF, IG and IH are preferred.

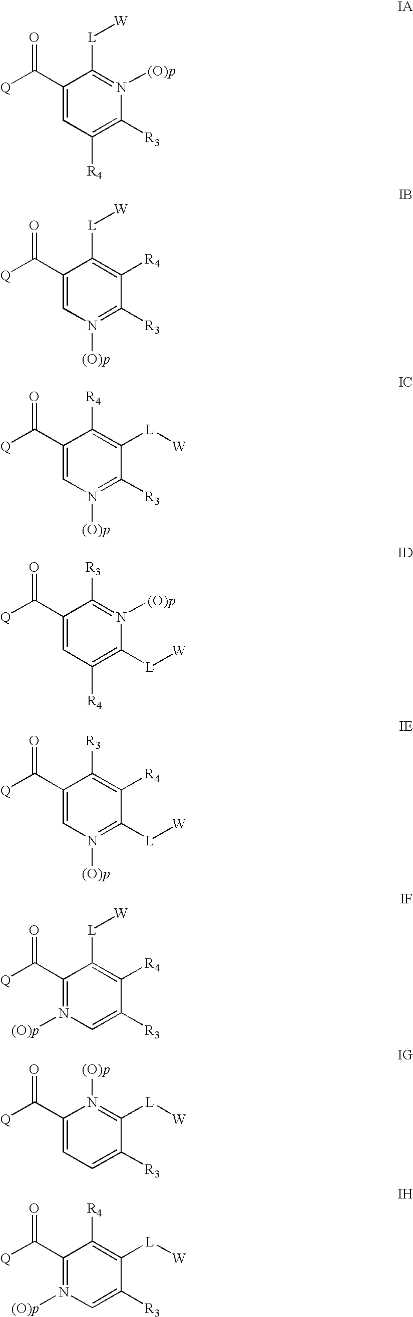

Special preference is given to the compounds of formula IA.

Of the compounds of formula I, special preference is given to those wherein W, as a 4- to 7-membered, saturated, partially saturated or unsaturated ring system U 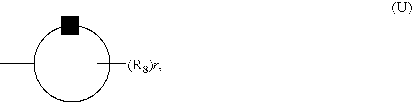
is a group bonded to L by way of the nitrogen atom adjacent to the ring element U₁ and is accordingly a cyclic group U₀ mono- or poly-substituted by R₈ 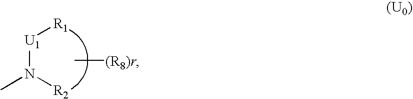
wherein R₁ together with R₂, by way of the nitrogen atom and the group U₁, forms the corresponding ring system U and wherein U₁, R₈ and r are as defined above.

Of the compounds of formula I and especially of the compounds of formula IA, special preference is given in turn to those groups wherein:
a) Q is a group Q₁, A₁ is CR₁₁R₁₂ and R₁₁ is hydrogen, methyl, ethyl, propargyl, methoxy-carbonyl, ethoxycarbonyl, methylthio, methylsulfinyl or methylsulfonyl and R₁₂ is hydrogen or methyl, or R₁₁ together with R₁₂ forms an ethylene bridge —(CH₂)₂—;
b) Q is a group Q₁ and A₂ is CR₁₄R₁₅ or an ethylene bridge —(CH₂)₂—, and R₁₄ is hydrogen, methyl or trifluoromethyl and R₁₅ is hydrogen or methyl, or R₁₄ together with R₁₁, or R₁₄ together with R₁₇ forms a direct bond or a methylene bridge;
c) Q is a group Q₁ and A₂ is C(O) and R₁₁, R₁₂, R₁₇ and R₁₈ are each methyl;
d) Q is a group Q₁ and A₂ is oxygen and R₁₁, R₁₂, R₁₇ and R₁₈ are each hydrogen or methyl;
e) Q is a group Q, and A₃ is CR₁₇R₁₈ and R₁₇ and R₁₈ are hydrogen or methyl, or R₁₇ together with R₁, forms a methylene or ethylene bridge;
f) Q is a group Q, and X₁ is hydroxy;
g) Q is a group Q₂ and R₂₁ is methyl or ethyl and R₂₂ is hydrogen or methyl;
h) Q is a group Q₂ and X₂ is hydroxy;
i) Q is a group Q₃ or Q₄ and R₃₂ is hydrogen, methylthio or methylsulfinyl, and R₃₁ and R₄₁ are cyclopropyl;
j) p is 0;
k) R₄ is hydrogen, methyl, chlorine or trifluoromethyl, especially hydrogen;
l) R₃ is C₁-C₃haloalkyl, especially difluoromethyl, chlorodifluoromethyl or trifluoromethyl;
m) L is either a direct bond or an unsubstituted C₁-C₃alkylene group or a C₁-C₃alkylene group uninterrupted or interrupted by oxygen, such as especially a methylene group —CH₂— or an ethylenemethoxymethylene group —CH₂OCH₂CH₂—;
n) R₁ and R₂ in the group —N(R₂)U₁R₁ form a 4- to 6-membered, saturated or partially saturated ring system which may additionally be substituted from one to three times by —N(R_{8b})—, once by oxygen, once by sulfur, sulfinyl or sulfonyl and/or once by a further carbonyl group;
o) U₁ is preferably a —C(═O)— group, a —C(═S)— group, a C(═NR₆)— group or a —SO₂— group;
p) the group —N(R₂)U₁R₁ is 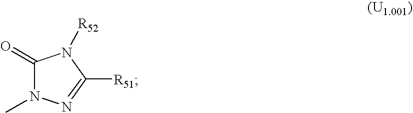
q) the group —N(R₂)U₁R₁ is 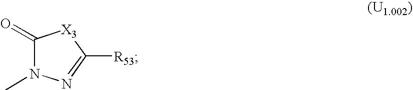
r) the group —N(R₂)U₁R₁ is 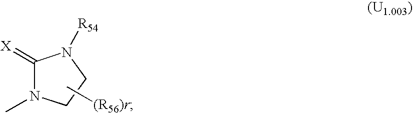
s) the group —N(R₂)U₁R₁ is 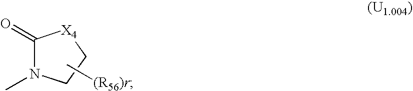
t) the group —N(R₂)U₁R₁ is 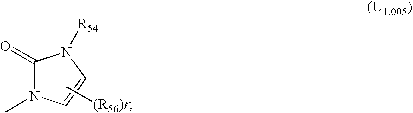
   u) the group —N(R₂)U₁R₁ is 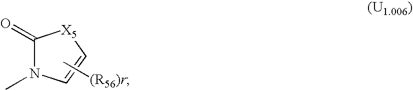
v) the group —N(R₂)U₁R₁ is 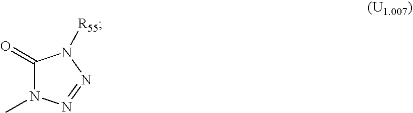
w) the group —N(R₂)U₁R₁ is a group selected from 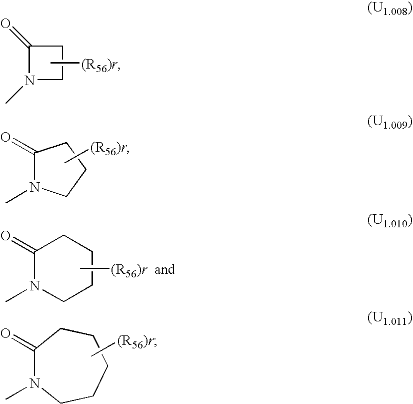
x) the group —N(R₂)U₁R₁ is 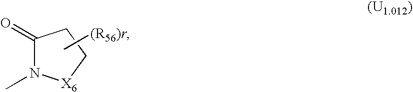
   wherein X₆ is oxygen or sulfur;
y) the group —N(R₂)U₁R₁ is 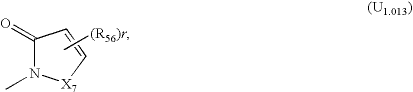
   wherein X₇ is oxygen or sulfur;
   z) the group —N(R₂)U₁R₁ is 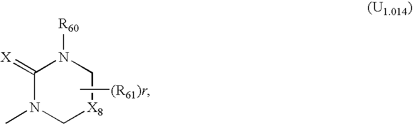
      wherein X is oxygen or sulfur and X₈ is —CH₂—;
aa) the group —N(R₂)U₁R₁ is 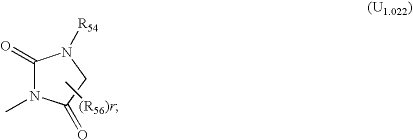
bb) the group —N(R₂)U₁R₁ is 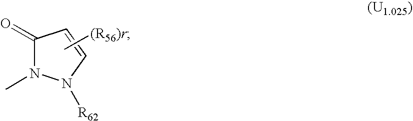
cc) the group —N(R₂)U₁R₁ is 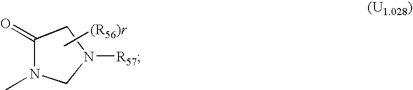
dd) the group —N(R₂)U₁R₁ is 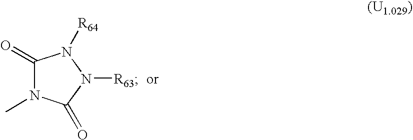
ee) the group —N(R₂)U₁R₁ is

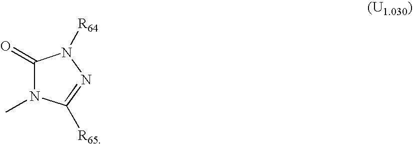

Special preference is given to the compounds of formula IA 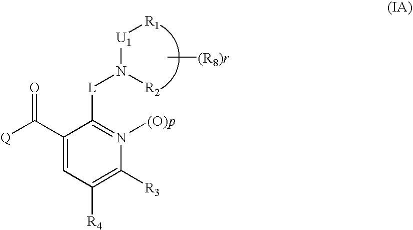
wherein Q, L, U₁, R₁, R₂, R₈ and r are as defined above and R₃ is difluoromethyl, chlorodifluoromethyl or trifluoromethyl, R₄ is hydrogen and p is 0.

The compounds of formula I can be prepared by means of processes known per se, as described below using the example of compounds of formula IA 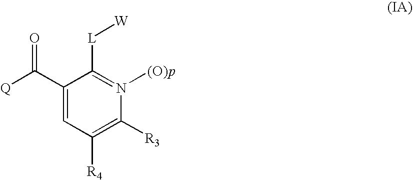
wherein W is a heterocyclic group U₀ 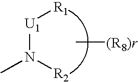
or, simplified, 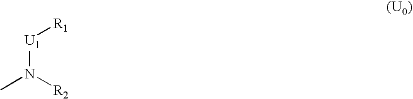
and wherein the group -L-N(R₂)U₁R₁ is located in the 2-position of the nicotinoyl group. In a preferred process, for example for the preparation of a compound of formula IA 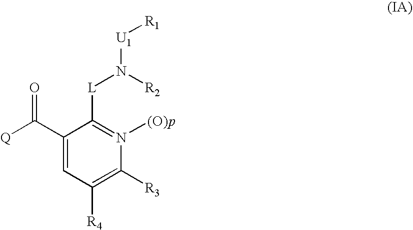
wherein L, U₁, R₁, R₂, R₃, R₄ and p are as defined above and Q is a group Q₁, Q₂ or Q₄, a compound of formula IIA 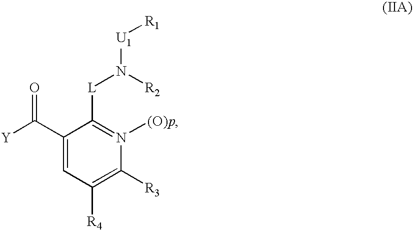
wherein L, U₁, R₁, R₂, R₃, R₄ and p are as defined above and Y is chlorine or cyano, is reacted in the presence of a base with a keto compound of formula IIIa, IIIb or IIId 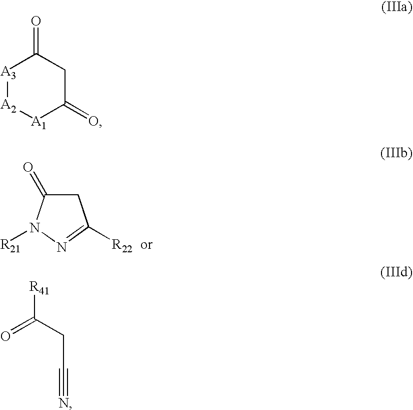
wherein A₁, A₂, A₃, R₂₁, R₂₂ and R₄₁, are as defined above, thus yielding the compound of formula IA directly in situ or yielding a compound of formula IVA 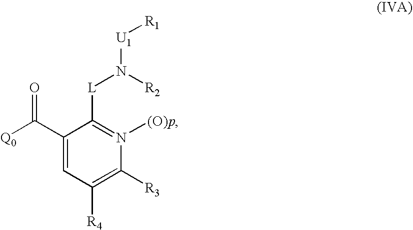
wherein L, U₁, R₁, R₂, R₃, R₄ and p are as defined above and Q₀ is accordingly the group Q linked to oxygen, which compound, especially when Y is chlorine, is then rearranged in the presence of an additional amount of cyanide ions, e.g. potassium cyanide, trimethylsilyl cyanide or acetone cyanohydrin, and in the presence of a base, e.g. triethylamine, to form a C-C-linked compound IA.

That process is illustrated by way of example with respect to compounds of formula IA wherein Q is a group Q₁, that is to say with respect to compounds of formula IAa, in Scheme 1.

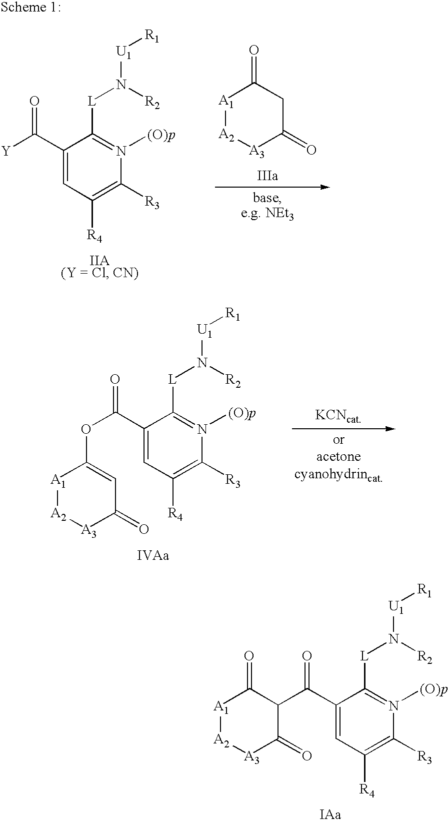

In a variant of that process, for example for the preparation of a compound of formula IA 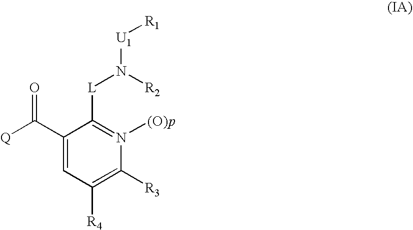
wherein L, U₁, R₁, R₂, R₃, R₄ and p are as defined above and Q is a group Q₁, Q₂ or Q₄, a compound of formula IIAd 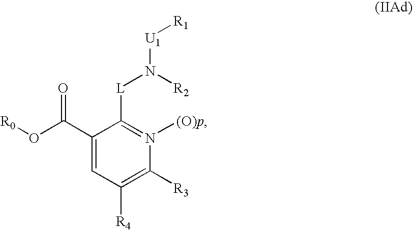
wherein L, U₁, R₁, R₂, R₃, R₄ and p are as defined above and R₀ is hydroxy, is reacted with the aid of a coupling reagent, for example dicyclohexylcarbodiimide, (1-chloro-2-methyl-propenyl)-dimethylamine or 2-chloro-1-methylpyridinium iodide, in the presence of a base, e.g. triethylamine or Hünig base, with a keto compound of formula IIIa, IIIb or IIId, respectively, 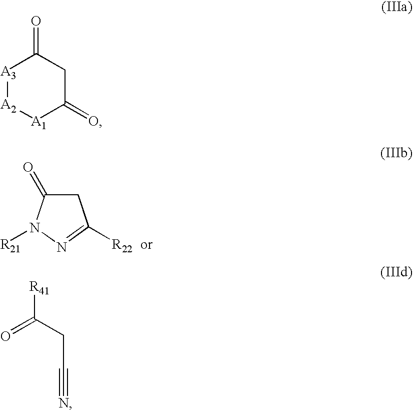
wherein A₁, A₂, A₃, R₂₁, R₂₂ and R₄₁ are as defined above, optionally via an intermediate of an activated ester of formula IIAe 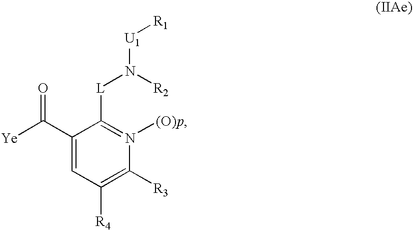
wherein L, U₁, R₁, R₂, R₃, R₄ and p are as defined above and the meaning of Ye depends upon the coupling reagent used, to form a compound of formula IVA 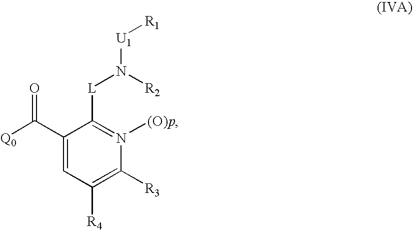
wherein L, U₁, R₁, R₂, R₃, R₄ and p are as defined above and Q₀ is accordingly the group Q linked to oxygen, and that compound is then, after isolation in a second reaction step or directly in situ, rearranged in the presence of a base, e.g. triethylamine, and a catalytic amount of cyanide ions, e.g. potassium cyanide or acetone cyanohydrin, or a catalytic amount of dimethylaminopyridine, to form a C-C-linked compound IA.

That process is illustrated by way of example with respect to compounds of formula IA wherein Q is a group Q., that is to say with respect to compounds of formula IAa, in Scheme 2.

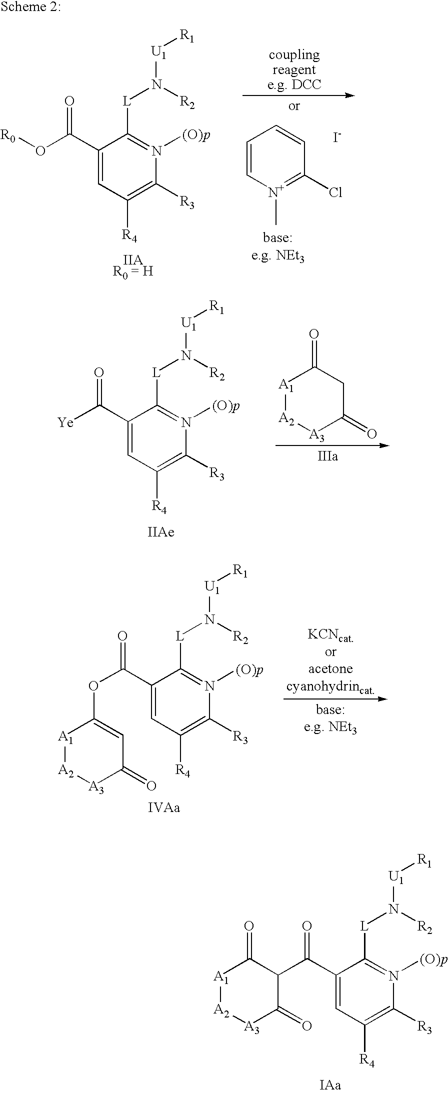

In a further process for the preparation of compounds of formula IA, a compound of formula VA 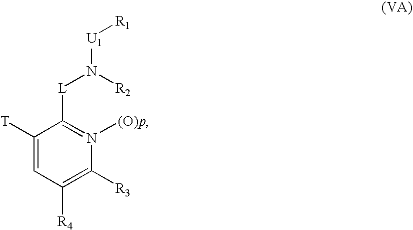
wherein L, U₁, R₁, R₂, R₃, R₄ and p are as defined above and T is chlorine, bromine, iodine or trifluoromethanesulfonyloxy, is reacted under carbonylation conditions, as described, for example, in Tetrahedron Letters, 31, 2841, 1990 and in WO 02/16305, in the presence of noble metal catalysts and suitable phosphine ligands, e.g. Pd(PPh₃)₄ or Pd(PPh₃)₂Cl₂, and suitable bases, e.g. triethylamine, with a compound of formula II, for example of formula IIIa or IIIb 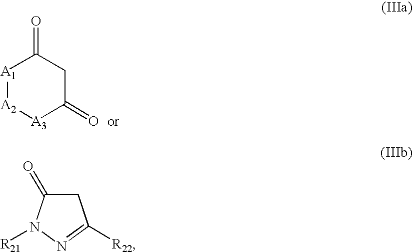
wherein A₁, A₂, A₃, R₂₁ and R₂₂ are as defined above, as illustrated in Scheme 3 for compounds of formula IAa wherein X₁ is hydroxy.

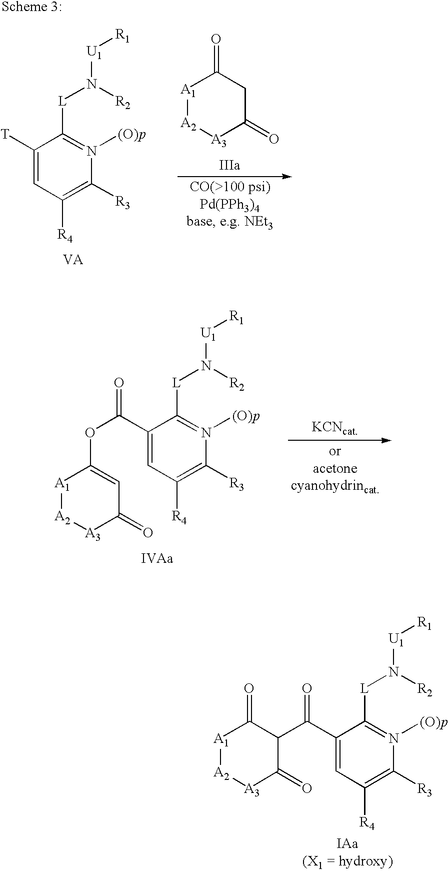

Compounds of formula IA 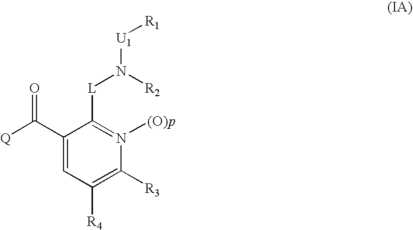
wherein L, U₁, R₁, R₂, R₃, R₄ and p are as defined above and Q is a group Q₃ 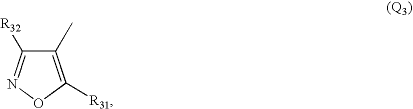
that is to say compounds of formula IAc, can likewise be prepared analogously to known procedures (for example analogously to the procedures described in WO 00/15615, WO 00/39094 and WO 01/94339), for example as follows: when X₃ is oxygen and R₃₂ is a group S(O)ₙR₃₃ wherein R₃₃ is as defined above, a compound of formula IIA 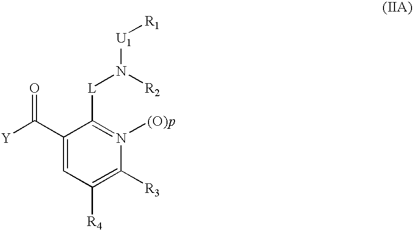
wherein L, U₁, R₁, R₂, R₃, R₄ and p are as defined above and Y is chlorine is converted in a Claisen condensation with a ketocarboxylic acid salt of formula XIV
R₃₁C(O)CH₂COO⁻M⁺ (XIV)
or with a trialkyl silyl ester of formula XIVa
R₃₁C(O)CH₂COOSi(R′R″R′″)₃ (XIVa),
wherein R₃₁ is as defined above and M⁺ is a metal salt cation, e.g. Li⁺ or K⁺, and R′, R″, R′″ are an alkyl group, e.g. methyl, into a compound of formula IIAa 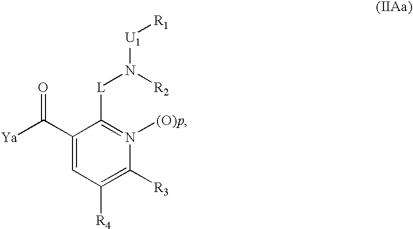
wherein L, U₁, R¹, R₂, R₃, R₄ and p are as defined above and Ya is CH₂C(O)R₃₁, that compound is then treated in the presence of a base with carbon disulfide and an alkylating reagent of formula XV
R₃₃Y₂ (XV),
wherein R₃₃ is as defined for formula I and Y₂ is a leaving group, such as halogen or sulfonyloxy, and converted into a compound of formula IIAb 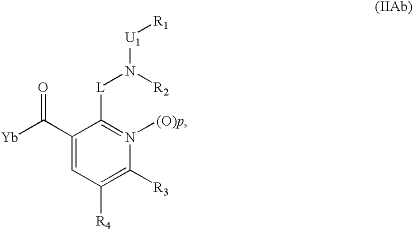
wherein L, U₁, R₁, R₂, R₃, R₄ and p are as defined above and Yb is a group Yb 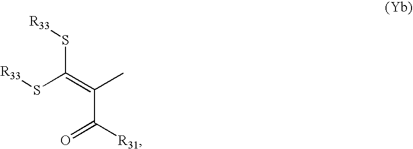
and then the compound of formula IIAb is cyclised with hydroxylamine hydrochloride and optionally in a solvent and in the presence of a base, for example sodium acetate, to form isomeric compounds of formula IAc and/or IAe, and the latter are then, when n is 1 or 2, oxidised with an oxidising agent, e.g. with a peracid, such as meta-chloroperbenzoic acid (m-CPBA) or peracetic acid, to form corresponding sulfoxides (n=1) or sulfones (n=2) of formula IAc 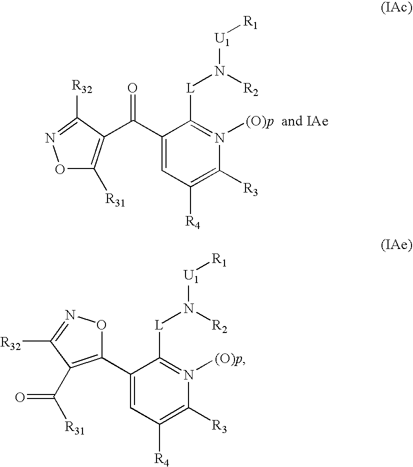
wherein L, U₁, R₁, R₂, R₃, R₄, R₃₁, and p are as defined above and R₃₂ is a group S(O)ₙR₃₃. That process is illustrated in Scheme 4.

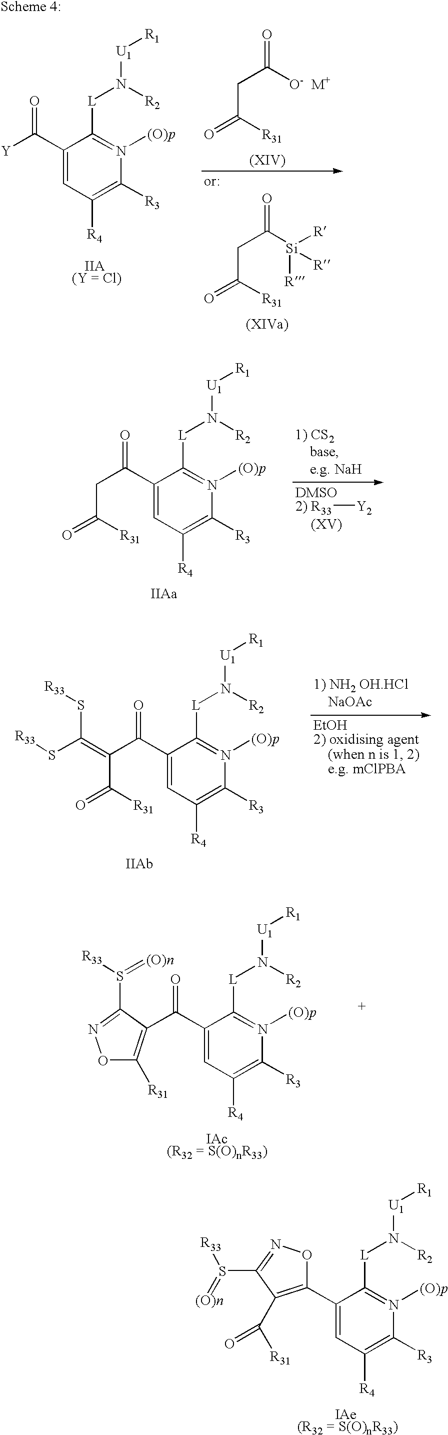

Compounds of formula IAc 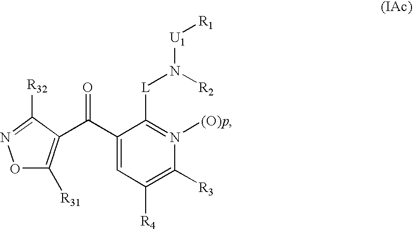
wherein L, U₁, R₁, R₂, R₃, R₄, R₃₁ and p are as defined above and R₃₂ is hydrogen, C₁-C₄-alkoxycarbonyl or carboxy, can likewise be prepared analogously to known procedures (e.g. analogously to the procedures described in WO 97/46530), for example as follows: a compound of formula IIAa 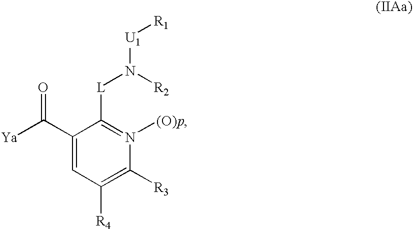
wherein L, U₁, R₁, R₂, R₃, R₄ and p are as defined above and Ya is CH₂C(O)R₃₁, is converted in the presence of a base with an ortho ester of formula XVI
R₃₂C(OR″)₂Y₃ (XVI)
or with a cyanic acid ester of formula XVII
R′″OC(O)CN (XVII),
wherein R₃₂ is hydrogen, Y₃ is a leaving group, such as C₁-C₄alkoxy or di(C₁-C₄alkyl)amino, and R′ and R′″ are C₁-C₄alkoxy, into a compound of formula IIAc 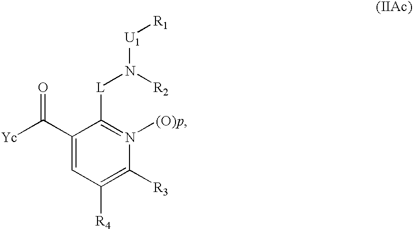
wherein L, U₁, R₁, R₂, R₃, R₄ and p are as defined above and Yc is a group Yc 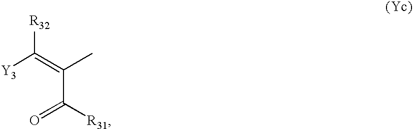
wherein R₃₁ is as defined above and R₃₂ is hydrogen or C₁-C₄alkoxycarbonyl and Y₃ is a leaving group, such as C₁-C₄alkoxy or di(C₁-C₄alkyl)amino, or hydroxy, and then the compound of formula IIAc is cyclised with hydroxylamine hydrochloride and optionally in a solvent and in the presence of a base, for example sodium acetate, to form isomeric compounds of formula IAc and/or IAe, and the latter are then, when R₃₂ is carboxyl or hydrogen, treated with a hydrolysing agent, e.g. with potassium hydroxide followed by a mineral acid, such as hydrochloric acid, to yield compounds of formula IAc 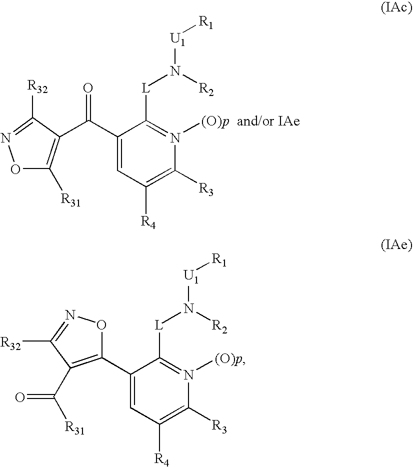
wherein L, U₁, R₁, R₂, R₃, R₄, R₃₁ and p are as defined above and R₃₂ is hydrogen, C₁-C₄-alkoxycarbonyl or carboxy. That process is illustrated in Scheme 5.

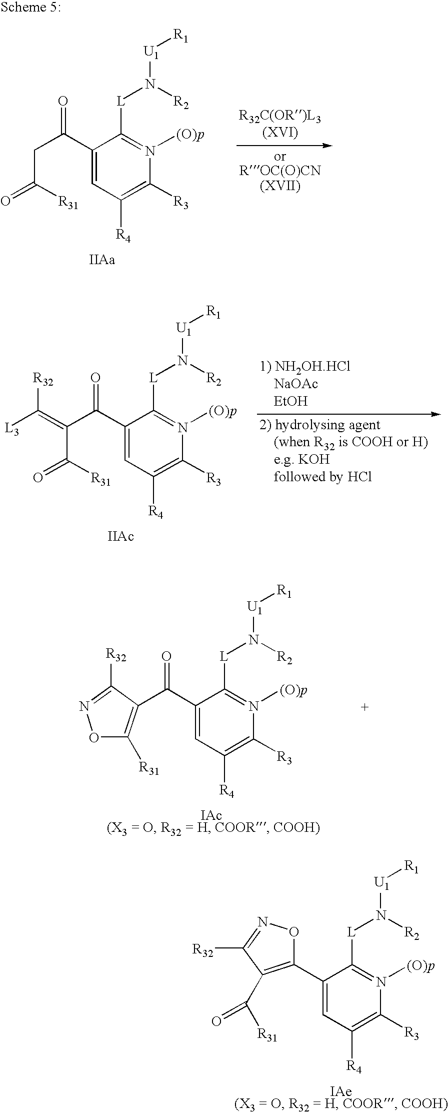

The isomeric compounds of formula IAc and IAe can be separated and purified, for example by means of column chromatography and a suitable eluant. In addition, compounds of formula IAe represent a sub-group of compounds of formula IA and accordingly the present invention relates likewise thereto.

Compounds of formula IA 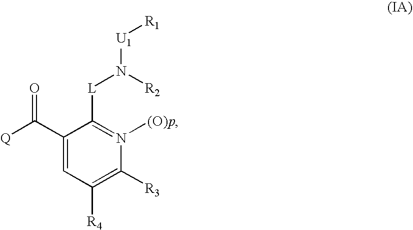
wherein L, U₁, R₁, R₂, R₃, R₄ and p are as defined above and X, or X₂ in the group Q₀ or Q₂, as the case may be, is S(O)ₙR₉ can likewise be prepared in accordance with known procedures by reacting a compound of formula IA wherein L, U₁, R₁, R₂, R₃, R₄ and p are as defined above and X₁ or X₂ in the group Q, or Q₂, respectively, is hydroxy, with a chlorinating agent, e.g. with oxalyl chloride, and then reacting the resulting compound of formula IA wherein L, U₁, R₁, R₂, R₃, R₄ and p are as defined above and X₁ or X₂ in the group Q₁ or Q₂, respectively, is chlorine, with a thio compound of formula VI
HSR₉ (VI)
or with a salt of formula VIa
M⁺⁻SR₉ (VIa),
wherein R₉ is as defined above, and optionally with an additional base, e.g. triethylamine, sodium hydride, sodium hydrogen carbonate or potassium carbonate, and for the preparation of a compound of formula IA wherein L, U₁, R₁, R₂, R₃, R₄ and p are as defined above and X₁ or X₂ in the group Q₁ or Q₂, respectively, is S(O)ₙR₉ and n is 1 or 2, treating the resulting compound of formula IA wherein L, U₁, R₁, R₂, R₃, R₄ and p are as defined above and X₁ or X₂ in the group Q₁ or Q₂, respectively, is SR₉, with an oxidising agent, e.g. sodium perbromate, sodium iodate, peracetic acid or m-chloroperbenzoic acid. That process sequence is illustrated in Scheme 6 using the example of compounds of formula IAa as defined above.

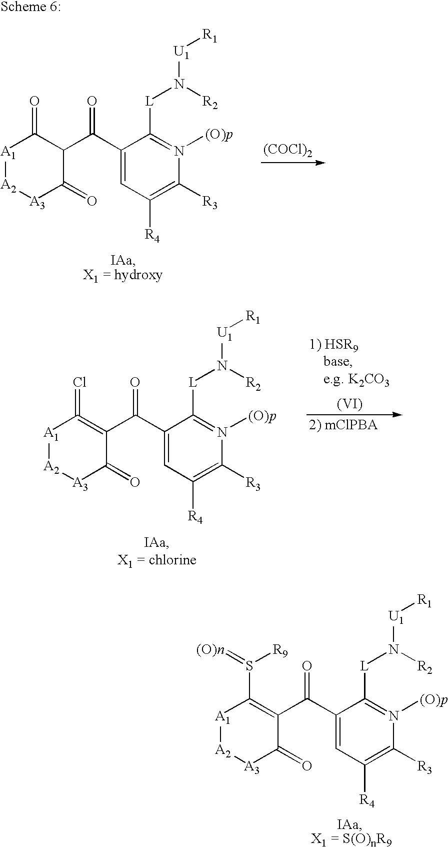

The compounds of formula IA 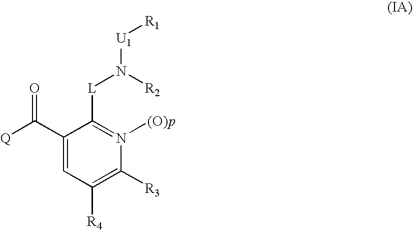
wherein Q, L, U₁, R₁, R₂, R₃, R₄ and p are as defined above can also be prepared by reacting a compound of formula XIIA 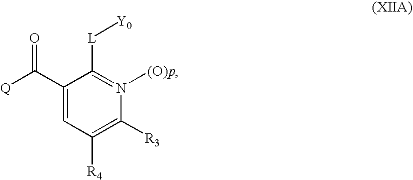
wherein Q, L, R₃, R₄ and p are as defined above and Y₀ is a leaving group, such as chlorine, bromine, mesyloxy or tosyloxy, with a corresponding amine compound of formula VIII
HN(R₂)U₁R₁ (VII)
or with a salt of formula VIIIa
M⁺⁻N(R₂)U₁R₁ (VIIIa)
wherein R₁, R₂ and U₁ are as defined above and M⁺ is a metal cation, it being possible to add a base, such as potassium carbonate, sodium hydride, sodium hydroxide, lithium hexa-methyldisilazane or lithium diisopropylamide. That general process is illustrated in Scheme 7.

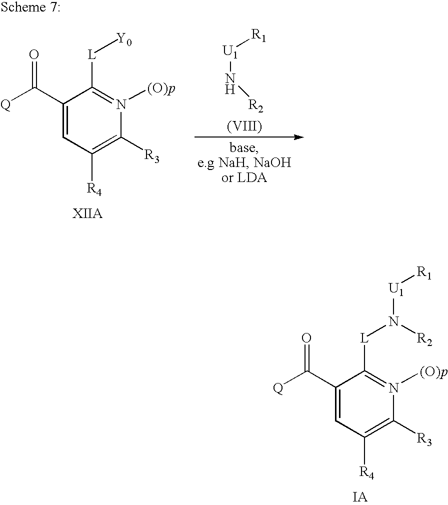

The compounds of formula IIA 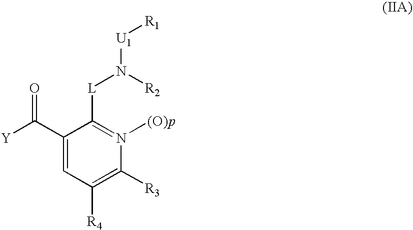
wherein L, U₁, R₁, R₂, R₃, R₄ and p are as defined above and Y is chlorine or cyano can be prepared by known methods from compounds of formula IIA wherein Y is hydroxy, C₁-C₄-alkoxy, benzyloxy, phenoxy or allyloxy, that is to say from compounds of formula IIAd 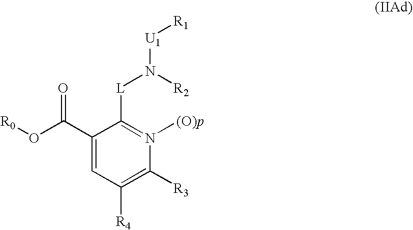
wherein L, U₁, R₀, R₁, R₂, R₃, R₄ and p are as defined above.

Such compounds of formula IIAa can be prepared, for example, from compounds of formula VIIA 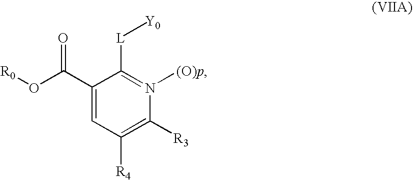
wherein L, R₀, R₃, R₄ and p are as defined above and Y₀ is a leaving group, such as chlorine, bromine, mesyloxy or tosyloxy, with a corresponding amino compound of formula VIII
HN(R₂)U₁R₁ (VIII)
or with a salt of formula VIIIa
M⁺⁻N(R₂)U₁R₁ (VIIIa)
wherein R₁, R₂ and U₁ are as defined above and M⁺ is a metal cation, it being possible to, add a base, such as potassium carbonate, sodium hydride, sodium hydroxide, potassium hydroxide, lithium hexamethyidisilazane or lithium diisopropylamide. That general process is illustrated in Scheme 8.

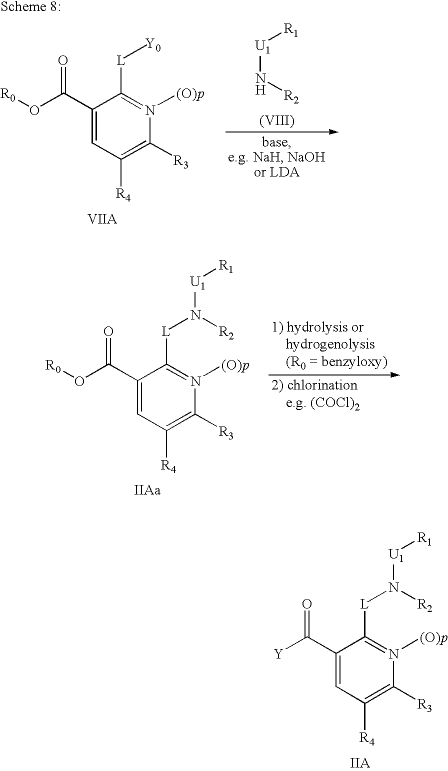

Compounds of formulae IIA and IIAa 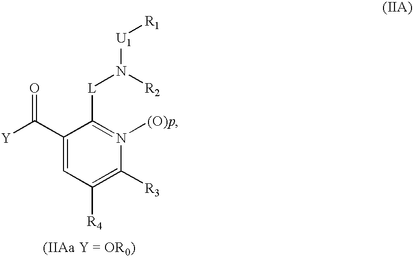
wherein L, U₁, R₀, R₁, R₂, R₄ and p are as defined above and R₃ is C₁-C₃haloalkyl can also be prepared by reacting a compound of formula IX 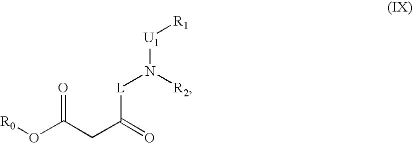
wherein L, U₁, R₀, R₁ and R₂ are as defined above, with an enamine of formula X 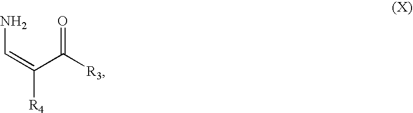
wherein R₄ is as defined above and R₃ is C₁-C₃haloalkyl, yielding a corresponding compound of formula IIAd 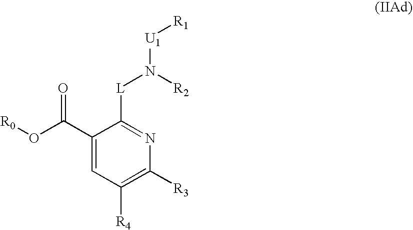
wherein L, U₁, R₀, R₁, R₂ and R₄ are as defined above and R₃ is C₁-C₃haloalkyl and p is 0, and that compound is then reacted further by generally known reaction methods for the conversion of the group R₀—O into a meaning of Y and optionally oxidation of the pyridyl nitrogen atom to the pyridyl-N-oxide, thus yielding a corresponding compound as defined above for formula IIA. That process is illustrated in Scheme 9.

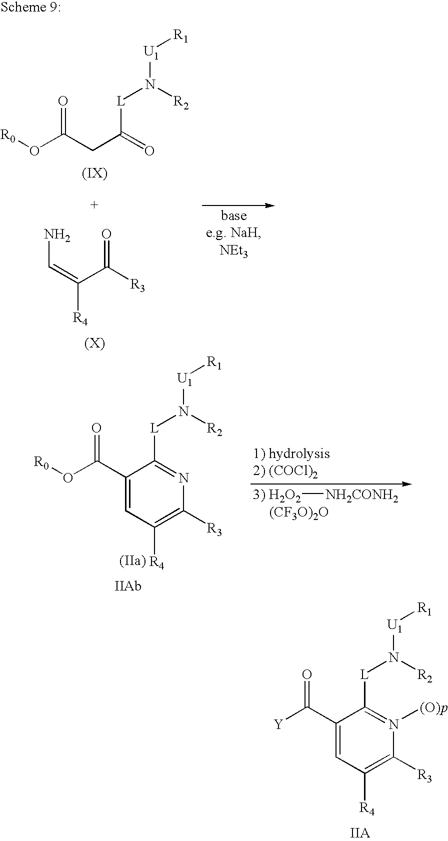

Compounds of formula IX can be prepared by reacting an acetoacetic acid ester of formula XI
R₀OC(O)CH₂C(O)CH₂Y₀ (XI),
wherein Y₀ is especially chlorine or bromine and R₀ is C₁-C₄alkoxy, with a corresponding amino compound of formula VIII.
HN(R₂)U₁R₁ (VIII)
or with a salt of formula VIIIa
M⁺⁻N(R₂)U₁R₁ (VIIIa),
wherein R₁, R₂ and U₁ are as defined above and M⁺ is a metal cation, the reaction advantageously being carried out in the presence of potassium carbonate, sodium hydride, sodium hydroxide, lithium hexamethyldisilazane or lithium diisopropylamide as acid-binding agent and base. That process is illustrated in Scheme 10.

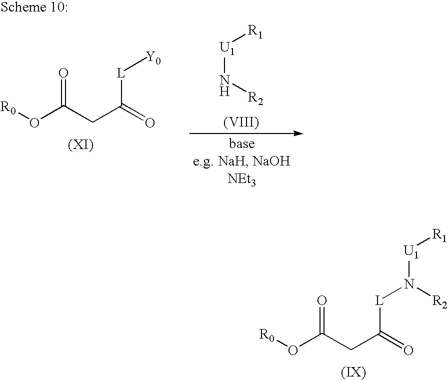

The compounds of formulae IIA, IIAa, IIAb, IIAc, IIAd, IVA and VA are valuable intermediates in the preparation of compounds of formula IA wherein R₃ is C₁-C₃haloalkyl and accordingly the present invention relates also thereto.

Those intermediates according to the invention are represented by the formula II 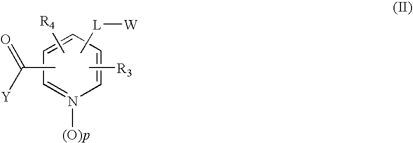
wherein Y is chlorine, cyano, hydroxy, C₁-C₄alkoxy, benzyloxy, phenoxy, allyloxy, a group 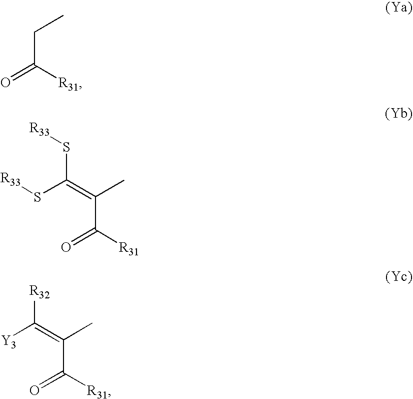
or a group Q₀, wherein Q₀ is accordingly a group Q linked to oxygen and Q, L, U₁, R₁, R₂, R₃, R₄, R₃₁, R₃₂, R₃₃ and p are as defined above for formula I.

The compounds of formula VII and especially compounds of formula VIIA are either known or can be prepared analogously to the methods described in WO 00/15615, WO 00/39094 and WO 01/94339. The compounds of formula XII and especially of formula XIIA are likewise known from the patent specifications mentioned above or can be prepared in accordance with the processes described therein.

The compounds of formula III used as starting materials are known or can be prepared in accordance with generally described methods, e.g. as described in the references mentioned above. The compounds of formula VIII are either known or can be prepared analogously to known methods, e.g. according to WO 99/18089.

All other compounds of formula I, such as especially those of formulae IB, IC, ID, IE, IF, IG and IH, can be prepared analogously to the processes described above.

The reactions to form compounds of formula I are advantageously carried out in aprotic, inert organic solvents. Such solvents are hydrocarbons, such as benzene, toluene, xylene or cyclohexane, chlorinated hydrocarbons, such as dichloromethane, trichloromethane, tetra-chloromethane or chlorobenzene, ethers, such as diethyl ether, ethylene glycol dimethyl ether, diethylene glycol dimethyl ether, tetrahydrofuran or dioxane, nitriles, such as aceto nitrile or propionitrile, amides, such as N,N-dimethylformamide, diethylformamide or N-methylpyrrolidinone. The reaction temperatures are preferably from −20° C. to +120° C. If the reactions proceed slightly exothermically, they can generally be carried out at room temperature. In order to shorten the reaction time or to initiate the reaction, brief heating, up to the boiling point of the reaction mixture, can be carried out. The reaction times can likewise be shortened by the addition of suitable bases as reaction catalysts. As bases there are used especially the tertiary amines, such as trimethylamine, triethylamine, quinuclidine, 2-methyl-4-ethylpyridine, dimethylaminopyridine, 1,4-diazabicyclo[2.2.2]octane, 1,5-diazabicyclo-[4.3.0]non-5-ene or 1,5-diazabicyclo[5.4.0]undec-7-ene. It is also possible, however, to use as bases inorganic bases, such as hydrides, e.g. sodium or calcium hydride, hydroxides, e.g. dry sodium or potassium hydroxide, carbonates, e.g. sodium or potassium carbonate, or hydrogen carbonates, e.g. sodium or potassium hydrogen carbonate.

According to Reaction Schemes 6, 8 and 9, the compounds of formulae I and II are prepared using a chlorinating agent, e.g. thionyl chloride, phosgene, phosphorus pentachloride, phosphorus oxychloride or preferably oxalyl chloride. The reaction is preferably carried out in an inert organic solvent, for example in aliphatic, halogenated aliphatic, aromatic or halogenated aromatic hydrocarbons, for example n-hexane, benzene, toluene, xylenes, dichloro-methane, 1,2-dichloroethane or chlorobenzene, at reaction temperatures in the range from −20° C. up to the reflux temperature of the reaction mixture, preferably at about from +40 to +100° C., and in the presence of a catalytic amount of N,N-dimethylformamide.

For the preparation of compounds of formulae I and IV according to Reaction Scheme 1 or with the aid of a coupling reagent, for example dicyclohexylcarbodiimide, (1-chloro-2-methyl-propenyl)-dimethylamine or 2-chloro-1-methylpyridinium iodide, according to Reaction Scheme 2, reaction is preferably likewise carried out in one of the inert organic solvents mentioned above at temperatures from about −20° C. to about +100° C., preferably from about +5° C. to about +50° C.

The end products of formula I can be isolated in conventional manner by concentration or evaporation of the solvent and purified by recrystallisation or trituration of the solid residue in solvents in which they are not readily soluble, such as ethers, aromatic hydrocarbons or chlorinated hydrocarbons, by distillation or by means of column chromatography or by means of the HPLC technique using a suitable eluant.

The sequence in which the reactions should be carried out in order as far as possible to avoid secondary reactions will also be familiar to the person skilled in the art. Unless the synthesis is specifically aimed at the isolation of pure isomers, the product may be obtained in the form of a mixture of two or more isomers, for example chiral centres in the case of alkyl groups or cis/trans isomerism in the case of alkenyl groups or <E> or <Z> forms, e.g. in respect of a —C(═NR₆)— group. All such isomers can be separated by methods known per se, for example chromatography, crystallisation, or produced in the desired form by means of a specific reaction procedure.

Compounds of formula I wherein p is 1, that is to say the corresponding pyridyl-N-oxides of formula I, can be prepared by reacting a compound of formula I wherein p is 0 with a suitable oxidising agent, for example with the H₂O₂ urea adduct in the presence of an acid anhydride, e.g. the trifluoroacetic anhydride. That reaction can be carried out either with compounds of formula I or at the stage of compounds of formula II, V, VII or XII.

For the use according to the invention of the compounds of formula I, or of compositions comprising them, there come into consideration all methods of application customary in agriculture, for example pre-emergence application, post-emergence application and seed dressing, and also various methods and techniques such as, for example, the controlled release of active ingredient. For that purpose a solution of the active ingredient is applied to mineral granule carriers or polymerised granules (urea/formaldehyde) and dried. If required, it is additionally possible to apply a coating (coated granules), which allows the active ingredient to be released in metered amounts over a specific period of time.

The compounds of formula I can be used as herbicides in unmodified form, that is to say as obtained in the synthesis, but they are preferably formulated in customary manner together with the adjuvants conventionally employed in formulation technology e.g. into emulsifiable concentrates, directly sprayable or dilutable solutions, dilute emulsions, suspensions, mixtures of a suspension and an emulsion (suspoemulsions), wettable powders, soluble powders, dusts, granules or microcapsules. Such formulations are described, for example, on pages 9 to 13 of WO 97/34485. As with the nature of the compositions, the methods of application, such as spraying, atomising, dusting, wetting, scattering or pouring, are selected in accordance with the intended objectives and the prevailing circumstances.

The formulations, that is to say the compositions, preparations or mixtures comprising the compound (active ingredient) of formula I or at least one compound of formula I and, usually, one or more solid or liquid formulation adjuvants, are prepared in known manner, e.g. by homogeneously mixing and/or grinding the active ingredients with the formulation adjuvants, for example solvents or solid carriers. Surface-active compounds (surfactants) may also be used in addition in the preparation of the formulations. Examples of solvents and solid carriers are given, for example, on page 6 of WO 97/34485.

Depending upon the nature of the compound of formula I to be formulated, suitable surface-active compounds are non-ionic, cationic and/or anionic surfactants and surfactant mixtures having good emulsifying, dispersing and wetting properties.

Examples of suitable anionic, non-ionic and cationic surfactants are listed, for example, on pages 7 and 8 of WO 97/34485.

In addition, the surfactants conventionally employed in formulation technology, which are described, inter alia, in “McCutcheon's Detergents and Emulsifiers Annual” MC Publishing Corp., Ridgewood N.J., 1981, Stache, H., “Tensid-Taschenbuch”, Carl Hanser Verlag, Munich/Vienna 1981, and M. and J. Ash, “Encyclopedia of Surfactants”, Vol. I-III, Chemical Publishing Co., New York, 1980-81, are also suitable for the preparation of the herbicidal compositions according to the invention.

The compositions according to the invention can additionally include an additive comprising an oil of vegetable or animal origin, a mineral oil, alkyl esters thereof or mixtures of such oils and oil derivatives.

The amounts of oil additive in the composition according to the invention is generally from 0.01 to 2%, based on the spray mixture. For example, the oil additive can be added to the spray tank in the desired concentration after the spray mixture has been prepared.

Preferred oil additives comprise mineral oils or an oil of vegetable origin, for example rapeseed oil, olive oil or sunflower oil, emulsified vegetable oil, such as AMIGO® obtainable from Rhone-Poulenc Canada Inc., alkyl esters of oils of vegetable origin, for example the methyl derivatives, or an oil of animal origin, such as fish oil or beef tallow. A preferred additive contains as active components essentially 80% by weight alkyl esters of fish oils and 15% by weight methylated rapeseed oil, and also 5% by weight of customary emulsifiers and pH modifiers.

Especially preferred oil additives comprise alkyl esters of higher fatty acids (C₈-C₂₂), especially the methyl derivatives of C₁₂-C₁₈fatty acids, for example the methyl esters of lauric acid, palmitic acid and oleic acid. Those esters are known as methyl laurate (CAS-111-82-0), methyl palmitate (CAS-112-39-0) and methyl oleate (CAS-112-62-9). A preferred fatty acid methyl ester derivative is Emery® 2230 and 2231 (Henkel subsidiary Cognis GMBH, DE)

The application and action of the oil additives can be improved by combining them with surface-active substances, such as non-ionic, anionic or cationic surfactants. Examples of suitable anionic, non-ionic and cationic surfactants are listed on pages 7 and 8 of WO 97/34485.

Preferred surface-active substances are anionic surfactants of the dodecylbenzylsulfonate type, especially the calcium salts thereof, and also non-ionic surfactants of the fatty alcohol ethoxylate type. Special preference is given to ethoxylated C₁₂-C₂₂fatty alcohols having a degree of ethoxylation of from 5 to 40. Examples of commercially available, preferred surfactants are the Genapol types (Clariant A G, Muttenz, Switzerland). Also preferred for use as surface-active substances are silicone surfactants, especially polyalkyl-oxide-modified heptamethyltrisiloxanes, such as are commercially available as e.g. Silwet L-77®, and also perfluorinated surfactants. The concentration of surface-active substances in relation to the total additive is generally from 1 to 30% by weight.

Examples of oil additives that consist of mixtures of oils or mineral oils or derivatives thereof with surfactants are Edenor ME SU®, Turbocharge® (Zeneca Agro, Stoney Creek, Ontario, Calif.) and Actipron® (BP Oil UK Limited, GB).

The addition of an organic solvent to the oil additive/surfactant mixture can also bring about a further enhancement of action. Suitable solvents are, for example, Solvesso® (ESSO) and Aromatic Solvent® (Exxon Corporation) types. The concentration of such solvents can be from 10 to 80% by weight of the total weight.

Such oil additives, which are also described, for example, in U.S. Pat. No. 4,834,908, are suitable for the composition according to the invention. A commercially available oil additive is known by the name MERGE®, is obtainable from the BASF Corporation and is essentially described, for example, in U.S. Pat. No. 4,834,908 in col. 5, as Example COC-1. A further oil additive that is preferred according to the invention is SCORE® (Novartis Crop Protection Canada.)

In addition to the oil additives listed above, in order to enhance the action of the compositions according to the invention it is also possible for formulations of alkyl pyrrolidones, such as are commercially available e.g. as Agrimax®, to be added to the spray mixture. Formulations of synthetic latices, such as, for example, polyacrylamide, polyvinyl compounds or poly-1-p-menthene, such as are commercially available as e.g. Bond®, Courier® or Emerald®, can also be used to enhance action. Solutions that contain propionic acid, for example Eurogkem Pen-e-trate®, can also be added as action-enhancing agent to the spray mixture.

The herbicidal formulations generally contain from 0.1 to 99% by weight, especially from 0.1 to 95% by weight, of herbicide, from 1 to 99.9% by weight, especially from 5 to 99.8% by weight, of a solid or liquid formulation adjuvant, and from 0 to 25% by weight, especially from 0.1 to 25% by weight, of a surfactant. Whereas commercial products will preferably be formulated as concentrates, the end user will normally employ dilute formulations. The compositions may also comprise further ingredients, such as stabilisers, for example vegetable oils or epoxidised vegetable oils (epoxidised coconut oil, rapeseed oil or soybean oil), anti-foams, for example silicone oil, preservatives, viscosity regulators, binders, tackifiers, and also fertilisers or other active ingredients.

The compounds of formula I are generally applied to plants or the locus thereof at rates of application of from 0.001 to 4 kg/ha, especially from 0.005 to 2 kg/ha. The concentration required to achieve the desired effect can be determined by experiment. It is dependent on the nature of the action, the stage of development of the cultivated plant and of the weed and on the application (place, time, method) and may vary within wide limits as a function of those parameters.

The compounds of formula I are distinguished by herbicidal and growth-inhibiting properties, allowing them to be used in crops of useful plants, especially cereals, cotton, soybeans, sugar beet, sugar cane, plantation crops, rape, maize and rice, and also for non-selective weed control.

The term ‘crops’ is to be understood as including also crops that have been rendered tolerant to herbicides or classes of herbicides (such as, for example, HPPD inhibitors, ALS inhibitors, EPSPS (5-enol-pyrovyl-shikimate-3-phosphate-synthase) inhibitors, GS (glutamine synthetase) inhibitors) as a result of conventional methods of breeding or genetic engineering. An example of a crop that has been rendered tolerant to imidazolinones, e.g. Imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady® and LibertyLink®.

The weeds to be controlled may be both monocotyledonous and dicotyledonous weeds, such as, for example, Stellaria, Nasturtium, Agrostis, Digitaria, Avena, Setaria, Sinapis, Lolium, Solanum, Echinochloa, Scirpus, Monochoria, Sagittaria, Bromus, Alopecurus, Sorghum halepense, Rottboellia, Cyperus, Abutilon, Sida, Xanthium, Amaranthus, Chenopodium, Ipomoea, Chrysanthemum, Galium, Viola and Veronica.

The following Examples further illustrate the invention but do not limit the invention.

### PREPARATION EXAMPLE P1

### 2-[6-(Chloro-difluoro-methyl)-3-(2-hydroxy-6-oxo-cyclohex-1-ene-carbonyl)-pyridin-2-ylmethyl]-4-methyl-5-trifluoromethyl-2.4-dihydro-[1.2.4]triazol-3-one:

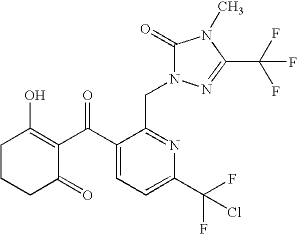 65 mg (0.17 mmol) of 6-(chloro-difluoro-methyl)-2-(4-methyl-5-oxo-3-trifluoromethyl-4,5-dihydro-[1.2.4]triazol-1-ylmethyl)-nicotinic acid (Preparation Example P6) are heated at 50° C. for 30 minutes in 5 ml of hexane with 0.02 ml of oxalyl chloride and a catalytic amount of dimethylformamide. The mixture is then concentrated by evaporation and taken up in 1 ml of acetonitrile, and the 6-(chloro-difluoro-methyl)-2-(4-methyl-5-oxo-3-trifluoromethyl-4,5-dihydro[1.2.4]triazol-1-ylmethyl)-nicotinic acid chloride so prepared is transferred into a solution of 60 mg (0.15 mmol) of cyclohexane-1,3-dione and 40 mg (0.4 mmol) of triethyl-amine in 2 ml of acetonitrile. After 40 minutes' stirring at room temperature, 1 drop of acetone cyanohydrin is added and stirring is continued for a further 2 hours. The reaction product is then taken up in ethyl acetate and washed once with dilute hydrochloric acid and once with sodium chloride solution, concentrated and purified by chromatography using the HPLC technique. Pure 2-[6-(chloro-difluoro-methyl)-3-(2-hydroxy-6-oxo-cyclohex-1-ene-carbonyl)-pyridin-2-ylmethyl]-4-methyl-5-trifluoromethyl-2,4-dihydro-[1.2.4]triazol-3-one is thus obtained in the form of a resin; ¹H-NMR (CDCl₃ in ppm relative to TMS): 16.96, b, 1H; 7.60, m, 2H, 5.18, s, 2H, 3.33, s, 3H, 2.82, m, 2H, 2.50, m, 2H, 2.19, m, 2H.

### PREPARATION EXAMPLE P2

### 3-[3-(2-Hydroxy-6-oxo-cyclohex-1-enecarbonyl)-6-trifluoromethyl-pyridin-2-ylmethyl]-5-methyl-3H-[1.3.4]oxadiazol-2-one:

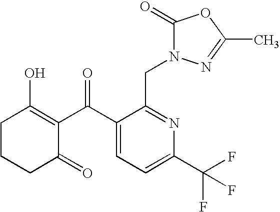

514 mg (1.694 mmol) of 2-(5-methyl-2-oxo-[1.3.4]oxadiazol-3-ylmethyl)-6-trifluoromethyl-nicotinic acid (Preparation Example P4) are introduced into 20 ml of dry methylene chloride. At 0° C., 0.264 ml (1.864 mmol) of (1-chloro-2-methyl-propenyl)-dimethyl-amine are squirted in and the mixture is then stirred at 20° C. for 2 hours. At 0° C., 0.190 g (1.694 mmol) of cyclo-hexane-1,3-dione and 0.354 ml (2.542 mmol) of triethylamine are then added and the mixture is stirred at 20° C. for 2 hours. The mixture is concentrated by evaporation and taken up in 20 ml of anhydrous acetonitrile, and 0.354 ml (2.542 mmol) of triethylamine and 0.155 ml (1.694 mmol) of acetone cyanohydrin are added to the reaction mixture. The reaction mixture is stirred at 20° C. for a further 20 hours and then concentrated by evaporation. The residue is purified by chromatography. The fractions are combined and concentrated. 0.570 g (84.7%) of pure 3-[3-(2-hydroxy-6-oxo-cyclohex-1-enecarbonyl)-6-trifluoromethyl-pyridin-2-ylmethyl]-5-methyl-3H-[1.3.4]oxadiazol-2-one is thus obtained in the form of a beige solid; ¹H-NMR (CDCl₃ in ppm relative to TMS): 17.6, b, 1H, 7.65, m, 2H, 4.98, s, 2H, 2.84, m, 2H, 2.48, m, 2H, 2.20, s, 3H, 2.08, m, 2H.

### PREPARATION EXAMPLE P3

### 3-{2-[3-(2-Hydroxy-4-oxo-bicyclo[3.2.1]oct-2-ene-3-carbonyl)-6-trifluoromethyl-pyridin-2-ylmethoxy]-ethyl}-5-methyl-3H-[1.3.4]thiadiazol-2-one

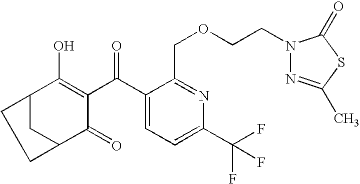

71 mg (1.635 mmol) of sodium hydride in the form of a 55% dispersion in oil are introduced into 2 ml of dry DMF. At 0° C., a solution of 300 mg (0.743 mmol) of 3-[2-(2-chloro-ethoxy-methyl)-6-trifluoromethyl-pyridine-3-carbonyl]-4-hydroxy-bicyclo[3.2.1]oct-3-en-2-one in 4 ml of anhydrous DMF is added dropwise. The reaction mixture is stirred at room temperature for 2 hours. In parallel, a further 71 mg (1.635 mmol) of sodium hydride in the form of a 55% dispersion in oil are introduced into a second flask and, at 0° C., 95 mg (0.817 mmol) of 5-methyl-3H-[1.3.4]thiadiazol-2-one are added. This mixture is also stirred at room temperature for 2 hours. Then, at the same temperature, the contents of the second flask are rapidly added to the reaction mixture in the first flask. The combined reaction mixture is then stirred at 20° C. for 4 hours and at 80° C. for 16 hours. The reaction product is poured into water and extracted with ethyl acetate. The organic phases are washed once with sodium chloride solution, dried over sodium sulfate and concentrated. The residue is purified by chromatography. 200 mg (55.7%) of pure 3-{2-[3-(2-hydroxy-4-oxo-bicyclo[3.2.1]oct-2-ene-3-carbonyl)-6-trifluoromethyl-pyridin-2-ylmethoxy]-ethyl}-5-methyl-3H-[1.3.4]thiadiazol-2-one are thus obtained in the form of a resin; ¹H-NMR (CDCl₃ in ppm relative to TMS): 16.9, b, 1H; 7.6, m, 2H, 4.72, s, 2H, 3.87, t, 2H, 3.62, t, 2H, 3.15, m, 1H, 2.87, m, 1H, 2.35, s, 3H, 2.3-2.0, m, 4H, 1.75, m, 2H.

### PREPARATION EXAMPLE P4

### 2-(5-Methyl-2-oxo-[1.3.4]oxadiazol-3-ylmethyl)-6-trifluoromethyl-nicotinic acid

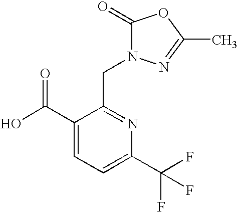

500 mg (1.509 mmol) of 2-(5-methyl-2-oxo-[1.3.4]oxadiazol-3-ylmethyl)-6-trifluoromethyl-nicotinic acid ethyl ester (Preparation Example P5) are introduced into 40 ml of a 1:1 mixture of THF/water at room temperature. At 0° C., 69.7 mg (1.66 mmol) of LiOH.H₂O are added. The reaction mixture is then stirred at the same temperature for 30 minutes. The reaction product is then extracted with ethyl acetate, washed with saturated sodium chloride solution, dried over sodium sulfate and concentrated by evaporation, yielding 420 mg (92%) of 2-(5-methyl-2-oxo-[1.3.4]oxadiazol-3-ylmethyl)-6-trifluoromethyl-nicotinic acid in the form of a white solid; ¹H-NMR (CD₃CN in ppm relative to TMS): 8.55, d, 1H; −7.82, d, 1H, 5.39, s, 2H; 2.20, s, 3H.

### PREPARATION EXAMPLE P5

### 2-(5-Methyl-2-oxo-[1.3.4]oxadiazol-3-ylmethyl)-6-trifluoromethyl-nicotinic acid ethyl ester

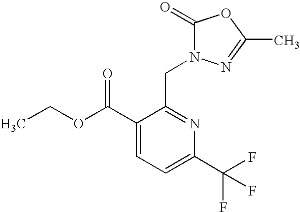

2.0 g (7.45 mmol) of 2-chloromethyl-6-trifluoromethyl-nicotinic acid ethyl ester are introduced into 8 ml of dry DMF at room temperature, and 1.0 g (8.19 mmol) of the sodium salt of 5-methyl-3H-[1.3.4]oxadiazol-2-one is added. The reaction mixture is then stirred at the same temperature for 20 hours. The reaction product is then diluted with water and extracted with ethyl acetate. The organic phases are washed once with sodium chloride solution, dried over sodium sulfate and concentrated. The residue is concentrated by evaporation and purified by chromatography, yielding 2.04 g (82%) of 2-(5-methyl-2-oxo-[1.3.4]oxadiazol-3-ylmethyl)-6-trifluoromethyl-nicotinic acid ethyl ester in the form of a white powder; ¹H-NMR (CDCl₃ in ppm relative to TMS): 8.48, d, 1H, 7.67, d, 1H, 5.45, s, 2H, 4.42, q, 2H, 2.26, s, 3H; 1;43, t, 3H.

### PREPARATION EXAMPLE P6

### 2-(3-Methyl-imidazolidin-2-on-1-ylmethyl)-6-trifluoromethylnicotinic acid

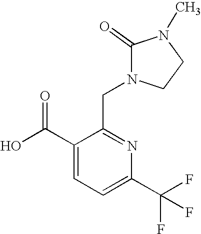

1.66 g (16.6 mmol) of 1-methyl-2-imidazolidinone are introduced into 50 ml of dry tetra-hydrofuran. At room temperature, 0.96 g (16.6 mmol) of pulverulent potassium hydroxide and 0.15 g (0.55 mmol) of 1,4,7,10,13,16-hexaoxacyclooctadecane are added thereto. The reaction mixture is stirred at room temperature for 2.5 hours. Then 1.48 g (5.53 mmol) of 2-chloromethyl-6-trifluoromethylnicotinic acid ethyl ester in 10 ml of dry tetrahydrofuran are added dropwise at room temperature in the course of 20 minutes. The reaction mixture is stirred at the same temperature for 22 hours. The reaction product is then diluted with water and extracted with ethyl acetate. The organic phases are washed with water. The aqueous phases are combined and rendered acidic with HCl (1 M solution). The aqueous phase is then extracted with ethyl acetate and the organic phases from the acidic extraction are combined, dried over sodium sulfate and concentrated. The residue is concentrated by evaporation, diluted with 8 ml of tetrabutyl methyl ether (TBME), stirred, filtered, concentrated, and dried under a high vacuum. 1.09 g of 2-(3-methyl-imidazolidin-2-on-1-ylmethyl)-6-trifluoromethylnicotinic acid are obtained in the form of a light-beige solid; ¹H-NMR (CD₃OD in ppm relative to TMS): 8.52, d, 1H, 7.78, d, 1H, 4.94, s, 2H, 3.65-3.35, 2×m, 2×2H, 2.82, s, 3H.

### PREPARATION EXAMPLE P7

### 6-(Chloro-difluoro-methyl)-2-(4-methyl-5-oxo-3-trifluoromethyl-4.5-dihydro-[1.2.4]triazol-1-ylmethyl)-nicotinic acid:

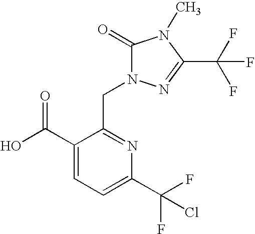

1 g (30 mmol) of 90% 4-(4-methyl-5-oxo-3-trifluoromethyl-4,5-dihydro-[1.2.4]triazol-1-yl)-3-oxo-butyric acid ethyl ester (Preparation Example P7) and 0.52 g (31 mmol) of 4-amino-1-chloro-1,1-difluoro-but-3-en-2-one are together heated at boiling temperature for 8 hours in 30 ml of toluene in the presence of 0.14 ml (1.8 mmol) of trifluoroacetic acid. The reaction product is then taken up in ethyl acetate and washed once with sodium hydrogen carbonate solution and once with sodium chloride solution. The residue is concentrated by evaporation and purified by chromatography, and 6-(chloro-difluoro-methyl)-2-(4-methyl-5-oxo-3-trifluoro-methyl-4,5-dihydro-[1.2.4]triazol-1-ylmethyl)-nicotinic acid ethyl ester is thus obtained in the form of an 80% product; ¹H-NMR (CDCl₃ in ppm relative to TMS): 8.45, d, 1H, 7.62, d, 1H; 5.65, s, 2H, 4.38, q, 2H, 3.45, s, 3H, 1.44, t, 3H.

The product is then hydrolysed in the presence of 1.4 equivalents of potassium hydroxide in a 1:1 mixture of dioxane/water at room temperature. The organic solvent and neutral secondary components are removed with diethyl ether and the aqueous phase is then acidified with hydrochloric acid and extracted with ethyl acetate. Pure 6-(chloro-difluoro-methyl)-2-(4-methyl-5-oxo-3-trifluoromethyl-4,5-dihydro-[1.2.4]triazol-1-ylmethyl)-nicotinic acid is thus obtained in the form of a crystalline product; ¹H-NMR (CDCl₃ in ppm relative to TMS): 10.42, b, 1H, 8.42, d, 1H, 7.61, d, 1H, 5.72, s, 2H, 3.50, s, 3H.

### PREPARATION EXAMPLE P8

### 4-(4-Methyl-5-oxo-3-trifluoromethyl-4.5-dihydro-[1.2.4]triazol-1-yl)-3-oxo-butyric acid ethyl ester

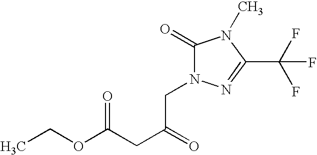

1.35 g (31 mol) of sodium hydride in the form of a 55% dispersion in oil are introduced into 30 ml of tetrahydrofuran. 2.55 g (15 mmol) of solid 4-methyl-5-trifluoromethyl-2,4-dihydro-[1.2.4]triazol-3-one hydroiodide are stirred in at room temperature and the mixture is briefly heated to 40° C. to complete the evolution of hydrogen. 1.95 ml (13.8 mmol) of 4-chloro-acetoacetic acid ethyl ester are then added dropwise to the resulting viscous suspension at a temperature of 20° C.; 4 drops of 15-crown-5 are added and the mixture is stirred at the same temperature for 16 hours. The reaction product is then poured into water and adjusted to pH 3 with hydrochloric acid, extracted with diethyl ether, washed with saturated sodium chloride solution and concentrated by evaporation. The residue is purified by chromatography (ethyl acetate/hexane gradient), 4-(4-methyl-5-oxo-3-trifluoromethyl-4,5-dihydro-[1.2.4]triazol-1-yl)-3-oxo-butyric acid ethyl ester being obtained in the form of a viscous oil; ¹H-NMR (CDCl₃ in ppm relative to TMS): 4.83, s, 2H, 4.22, q, 2H, 3.55, s, 2H, 3.39, s, 3H; 1.28, t, 3H.

All further compounds of formula I can be prepared analogously to the preparation methods and Examples described above.

In the following Tables, the linkage site of the individual structures of the group 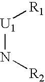
to the substituent L is the nitrogen atom located at the same geometric position, as indicated in each case.

For example, the linkage site of the group 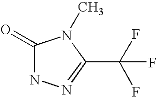
in the case of compound A 1.001 is the position indicated by an arrow:

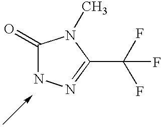

The free valencies in these structures are terminal CH₃ groups, such as, for example, in the case of the structure

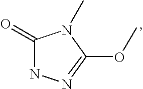

which can also be represented as follows: N

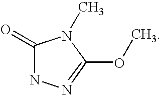

TABLE A1
Compounds of formula IAa₁:

| | |
|---|---|
| | (IAa₁) |
| 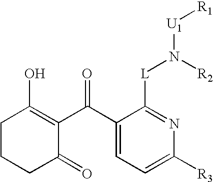 | |

| Comp. No. | R₃ | L | 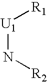 | Phys. data |
|---|---|---|---|---|
| A1.001 (P1) | CF₂Cl | 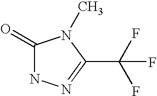 | resin | |
| A1.002 | CF₂H | CH₂ | 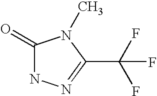 | |
| A1.003 | CF₃ | CH₂ | 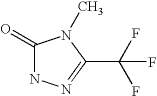 | |
| A1.004 | CF₃ | CH₂OCH₂CH₂ | 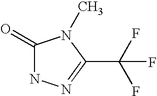 | |
| A1.005 | CF₂Cl | CH₂OCH₂CH₂ | 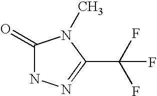 | |
| A1.006 | CHF₂ | CH₂OCH₂CH₂ | 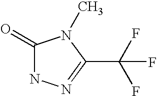 | |
| A1.007 | CF₃ | CH₂ | 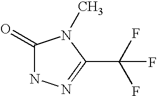 | solid |
| A1.008 | CF₂Cl | CH₂ | 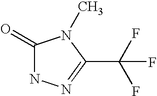 | |
| A1.009 | CHF2 | CH₂ | 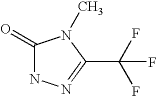 | |
| A1.010 | CF₃ | CH₂OCH₂CH₂ | 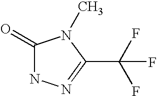 | |
| A1.011 | CF₂Cl | CH₂OCH₂CH₂ | 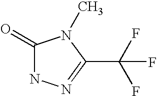 | |
| A1.012 | CHF₂ | CH₂OCH₂CH₂ | 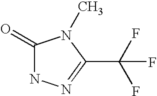 | |
| A1.013 | CF₃ | CH₂ | 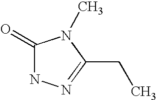 | |
| A1.014 | CF₂Cl | CH₂ | 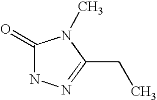 | |
| A1.015 | CHF₂ | CH₂ | 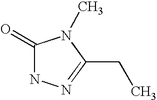 | |
| A1.016 | CF₃ | CH₂OCH₂CH₂ | 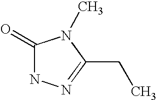 | |
| A1.017 | CF₂Cl | CH₂OCH₂CH₂ | 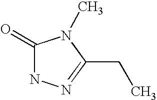 | |
| A1.018 | CHF₂ | CH₂OCH₂CH₂ | 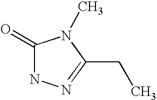 | |
| A1.019 | CF₃ | CH₂ | 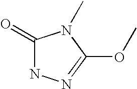 | solid |
| A1.020 | CF₂Cl | CH₂ | 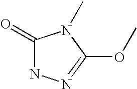 | |
| A1.021 | CHF₂ | CH₂ | 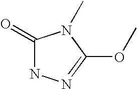 | |
| A1.022 | CF₃ | CH₂OCH₂CH₂ | 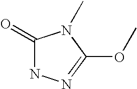 | |
| A1.023 | CF₂Cl | CH₂OCH₂CH₂ | 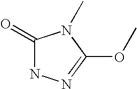 | |
| A1.024 | CHF₂ | CH₂OCH₂CH₂ | 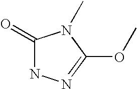 | |
| A1.025 | CF₃ | CH₂ | 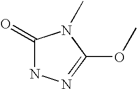 | solid |
| A1.026 | CF₂Cl | CH₂ | 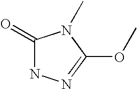 | |
| A1.027 | CHF₂ | CH₂ | 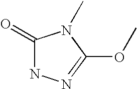 | |
| A1.028 | CF₃ | CH₂OCH₂CH₂ | 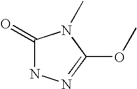 | |
| A1.029 | CF₂Cl | CH₂OCH₂CH₂ | 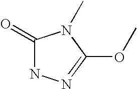 | |
| A1.030 | CHF₂ | CH₂OCH₂CH₂ | 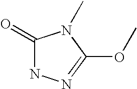 | |
| A1.031 | CF₃ | CH₂ | 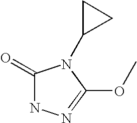 | solid |
| A1.032 | CF₂Cl | CH₂ | 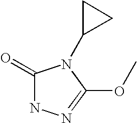 | |
| A1.033 | CHF₂ | CH₂ | 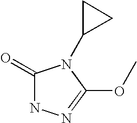 | |
| A1.034 | CF₃ | CH₂OCH₂CH₂ | 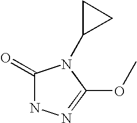 | |
| A1.035 | CF₂Cl | CH₂OCH₂CH₂ | 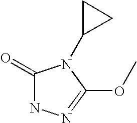 | |
| A1.036 | CHF₂ | CH₂OCH₂CH₂ | 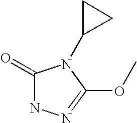 | |
| A1.037 | CF₃ | CH₂ | 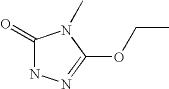 | solid |
| A1.038 | CF₂Cl | CH₂ | 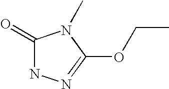 | |
| A1.039 | CHF₂ | CH₂ | 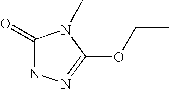 | |
| A1.040 | CF₃ | CH₂OCH₂CH₂ | 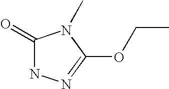 | |
| A1.041 | CF₂Cl | CH₂OCH₂CH₂ | 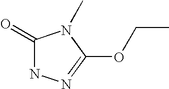 | |
| A1.042 | CHF₂ | CH₂OCH₂CH₂ | 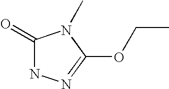 | |
| A1.043 | CF₃ | CH₂ | 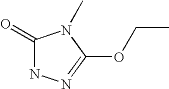 | resin |
| A1.044 | CF₂Cl | CH₂ | 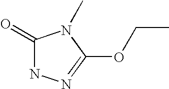 | |
| A1.045 | CHF₂ | CH₂ | 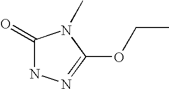 | |
| A1.046 | CF₃ | CH₂OCH₂CH₂ | 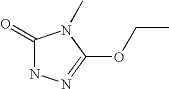 | |
| A1.047 | CF₂Cl | CH₂OCH₂CH₂ | 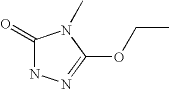 | |
| A1.048 | CHF₂ | CH₂OCH₂CH₂ | 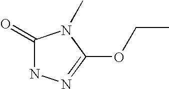 | |
| A1.049 | CF₃ | CH₂ | 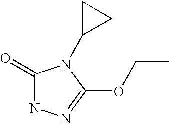 | resin |
| A1.050 | CF₂Cl | CH₂ | 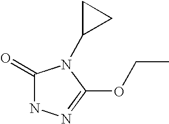 | |
| A1.051 | CHF₂ | CH₂ | 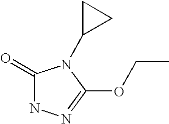 | |
| A1.052 | CF₃ | CH₂OCH₂CH₂ | 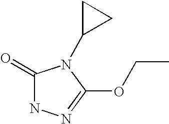 | |
| A1.053 | CF₂Cl | CH₂OCH₂CH₂ | 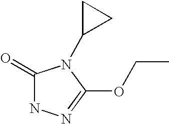 | |
| A1.054 | CHF₂ | CH₂OCH₂CH₂ | 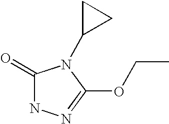 | |
| A1.055 | CF₃ | CH₂ | 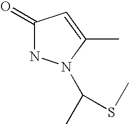 | resin |
| A1.056 | CF₂Cl | CH₂ | 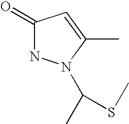 | |
| A1.057 | CHF₂ | CH₂ | 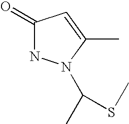 | |
| A1.058 | CF₃ | CH₂OCH₂CH₂ | 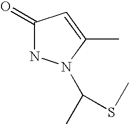 | |
| A1.059 | CF₂Cl | CH₂OCH₂CH₂ | 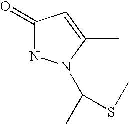 | |
| A1.060 | CHF₂ | CH₂OCH₂CH₂ | 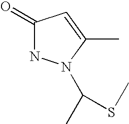 | |
| A1.061 | CF₃ | CH₂ | 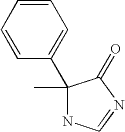 | |
| A1.062 | CF₂Cl | CH₂ | 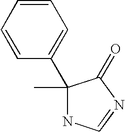 | |
| A1.063 | CHF₂ | CH₂ | 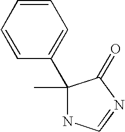 | |
| A1.064 | CF₃ | CH₂OCH₂CH₂ | 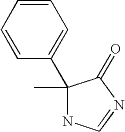 | |
| A1.065 | CF₂Cl | CH₂OCH₂CH₂ | 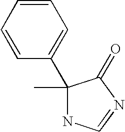 | |
| A1.066 | CHF₂ | CH₂OCH₂CH₂ | 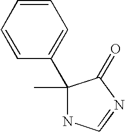 | |
| A1.067 | CF₃ | CH₂ | 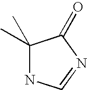 | |
| A1.068 | CF₂Cl | CH₂ | 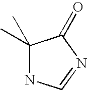 | |
| A1.069 | CHF₂ | CH₂ | 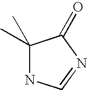 | |
| A1.070 | CF₃ | CH₂OCH₂CH₂ | 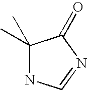 | |
| A1.071 | CF₂Cl | CH₂OCH₂CH₂ | 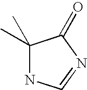 | |
| A1.072 | CHF₂ | CH₂OCH₂CH₂ | 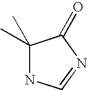 | |
| A1.073 | CF₃ | CH₂ | 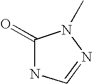 | m.p.: 140° C. |
| A1.074 | CF₂Cl | CH₂ | 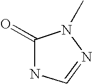 | m.p.: 125-127° C. |
| A1.075 | CHF₂ | CH₂ | 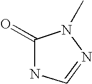 | |
| A1.076 | CF₃ | CH₂OCH₂CH₂ | 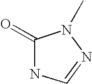 | |
| A1.077 | CF₂Cl | CH₂OCH₂CH₂ | 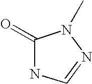 | |
| A1.078 | CHF₂ | CH₂OCH₂CH₂ | 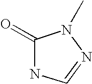 | |
| A1.079 | CF₃ | CH₂ | 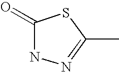 | amorphous crystals |
| A1.080 | CF₂Cl | CH₂ | 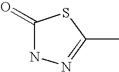 | |
| A1.081 | CHF₂ | CH₂ | 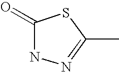 | |
| A1.082 | CF₃ | CH₂OCH₂CH₂ | 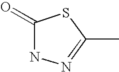 | resin |
| A1.083 | CF₂Cl | CH₂OCH₂CH₂ | 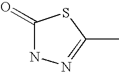 | |
| A1.084 | CHF₂ | CH₂OCH₂CH₂ | 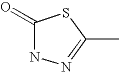 | |
| A1.085 | CF₃ | CH₂ | 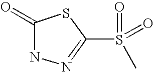 | amorphous crystals |
| A1.086 | CF₂Cl | CH₂ | 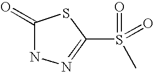 | |
| A1.087 | CHF₂ | CH₂ | 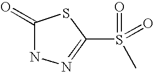 | |
| A1.088 | CF₃ | CH₂OCH₂CH₂ | 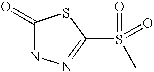 | |
| A1.089 | CF₂Cl | CH₂OCH₂CH₂ | 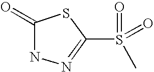 | |
| A1.090 | CHF₂ | CH₂OCH₂CH₂ | 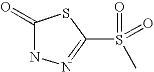 | |
| A1.091 | CF₃ | CH₂ | 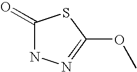 | resin |
| A1.092 | CF₂Cl | CH₂ | 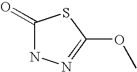 | |
| A1.093 | CHF₂ | CH₂ | 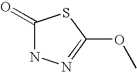 | |
| A1.094 | CF₃ | CH₂OCH₂CH₂ | 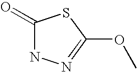 | |
| A1.095 | CF₂Cl | CH₂OCH₂CH₂ | 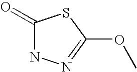 | |
| A1.096 | CHF₂ | CH₂OCH₂CH₂ | 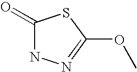 | |
| A1.097 (P2) | CF₃ | CH₂ | 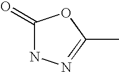 | amorphous crystals |
| A1.098 | CF₂Cl | CH₂ | 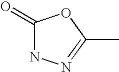 | m.p.: 130-132° C. |
| A1.099 | CHF₂ | CH₂ | 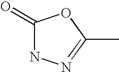 | |
| A1.100 | CF₃ | CH₂OCH₂CH₂ | 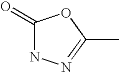 | resin |
| A1.101 | CF₂Cl | CH₂OCH₂CH₂ | 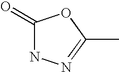 | |
| A1.102 | CHF₂ | CH₂OCH₂CH₂ | 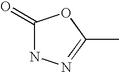 | |
| A1.103 | CF₃ | CH₂ | 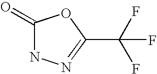 | resin |
| A1.104 | CF₂Cl | CH₂ | 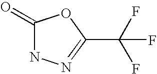 | |
| A1.105 | CHF₂ | CH₂ | 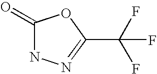 | |
| A1.106 | CF₃ | CH₂OCH₂CH₂ | 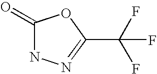 | |
| A1.107 | CF₂Cl | CH₂OCH₂CH₂ | 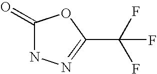 | |
| A1.108 | CHF₂ | CH₂OCH₂CH₂ | 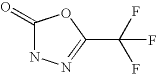 | |
| A1.109 | CF₃ | CH₂ | 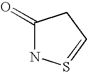 | resin |
| A1.110 | CF₂Cl | CH₂ | 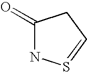 | |
| A1.111 | CHF₂ | CH₂ | 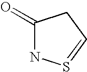 | |
| A1.112 | CF₃ | CH₂OCH₂CH₂ | 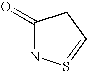 | |
| A1.113 | CF₂Cl | CH₂OCH₂CH₂ | 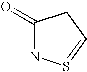 | |
| A1.114 | CHF₂ | CH₂OCH₂CH₂ | 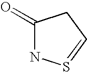 | |
| A1.115 | CF₃ | CH₂ | 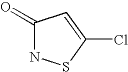 | resin |
| A1.116 | CF₂Cl | CH₂ | 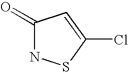 | |
| A1.117 | CHF₂ | CH₂ | 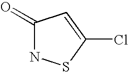 | |
| A1.118 | CF₃ | CH₂OCH₂CH₂ | 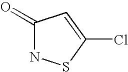 | |
| A1.119 | CF₂Cl | CH₂OCH₂CH₂ | 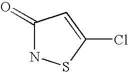 | |
| A1.120 | CHF₂ | CH₂OCH₂CH₂ | 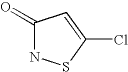 | |
| A1.121 | CF₃ | CH₂ | 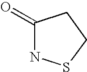 | |
| A1.122 | CF₂Cl | CH₂ | 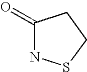 | |
| A1.123 | CHF₂ | CH₂ | 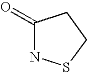 | |
| A1.124 | CF₃ | CH₂OCH₂CH₂ | 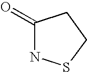 | |
| A1.125 | CF₂Cl | CH₂OCH₂CH₂ | 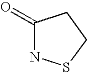 | |
| A1.126 | CHF₂ | CH₂OCH₂CH₂ | 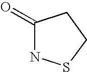 | |
| A1.127 | CF₃ | CH₂ | 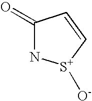 | |
| A1.128 | CF₂Cl | CH₂ | 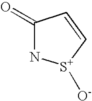 | |
| A1.129 | CHF₂ | CH₂ | 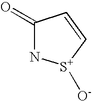 | |
| A1.130 | CF₃ | CH₂OCH₂CH₂ | 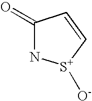 | |
| A1.131 | CF₂Cl | CH₂OCH₂CH₂ | 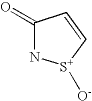 | |
| A1.132 | CHF₂ | CH₂OCH₂CH₂ | 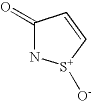 | |
| A1.133 | CF₃ | CH₂ | 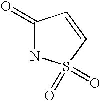 | |
| A1.134 | CF₂Cl | CH₂ | 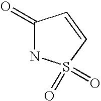 | |
| A1.135 | CHF₂ | CH₂ | 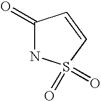 | |
| A1.136 | CF₃ | CH₂OCH₂CH₂ | 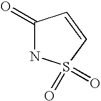 | |
| A1.137 | CF₂Cl | CH₂OCH₂CH₂ | 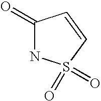 | |
| A1.138 | CHF₂ | CH₂OCH₂CH₂ | 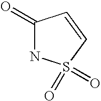 | |
| A1.139 | CF₃ | CH₂ | 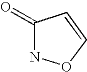 | |
| A1.140 | CF₂Cl | CH₂ | 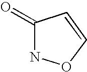 | |
| A1.141 | CHF₂ | CH₂ | 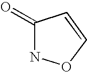 | |
| A1.142 | CF₃ | CH₂OCH₂CH₂ | 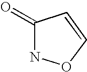 | |
| A1.143 | CF₂Cl | CH₂OCH₂CH₂ | 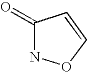 | |
| A1.144 | CHF₂ | CH₂OCH₂CH₂ | 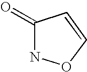 | |
| A1.145 | CF₃ | CH₂ | 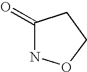 | |
| A1.146 | CF₂Cl | CH₂ | 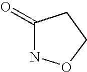 | |
| A1.147 | CHF₂ | CH₂ | 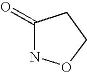 | |
| A1.148 | CF₃ | CH₂OCH₂CH₂ | 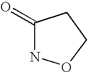 | |
| A1.149 | CF₂Cl | CH₂OCH₂CH₂ | 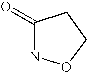 | |
| A1.150 | CHF₂ | CH₂OCH₂CH₂ | 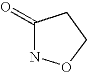 | |
| A1.151 | CF₃ | CH₂ | 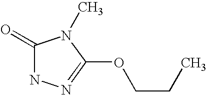 | |
| A1.152 | CF₂Cl | CH₂ | 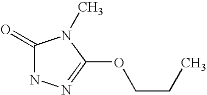 | |
| A1.153 | CHF₂ | CH₂ | 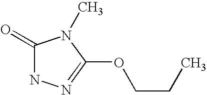 | |
| A1.154 | CF₃ | CH₂OCH₂CH₂ | 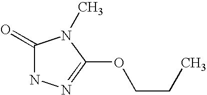 | |
| A1.155 | CF₂Cl | CH₂OCH₂CH₂ | 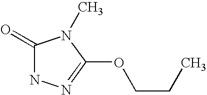 | |
| A1.156 | CHF₂ | CH₂OCH₂CH₂ | 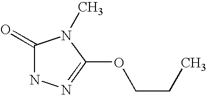 | |
| A1.157 | CF₃ | CH₂ | 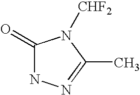 | |
| A1.158 | CF₂Cl | CH₂ | 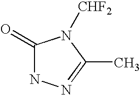 | |
| A1.159 | CHF₂ | CH₂ | 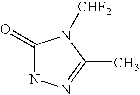 | |
| A1.160 | CF₃ | CH₂OCH₂CH₂ | 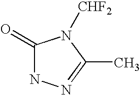 | |
| A1.161 | CF₂Cl | CH₂OCH₂CH₂ | 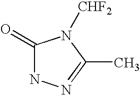 | |
| A1.162 | CHF₂ | CH₂OCH₂CH₂ | 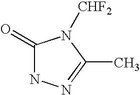 | |
| A1.163 | CF₃ | CH₂ | 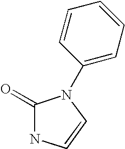 | |
| A1.164 | CF₂Cl | CH₂ | 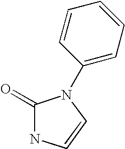 | |
| A1.165 | CHF₂ | CH₂ | 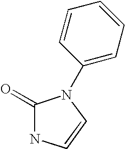 | |
| A1.166 | CF₃ | CH₂OCH₂CH₂ | 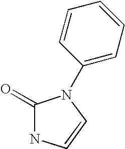 | |
| A1.167 | CF₂Cl | CH₂OCH₂CH₂ | 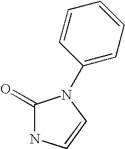 | |
| A1.168 | CHF₂ | CH₂OCH₂CH₂ | 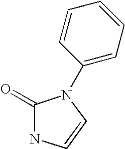 | |
| A1.169 | CF₃ | CH₂ | 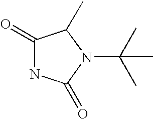 | |
| A1.170 | CF₂Cl | CH₂ | 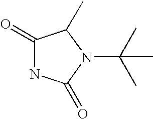 | |
| A1.171 | CHF₂ | CH₂ | 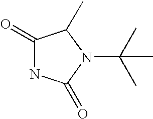 | |
| A1.172 | CF₃ | CH₂OCH₂CH₂ | 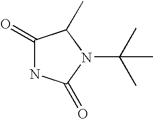 | |
| A1.173 | CF₂Cl | CH₂OCH₂CH₂ | 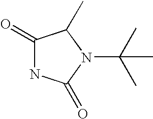 | |
| A1.174 | CHF₂ | CH₂OCH₂CH₂ | 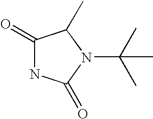 | |
| A1.175 | CF₃ | CH₂ | 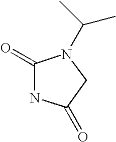 | m.p.: 141° C. |
| A1.176 | CF₂Cl | CH₂ | 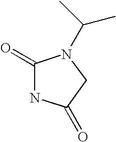 | |
| A1.177 | CHF₂ | CH₂ | 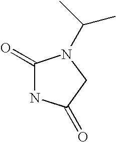 | |
| A1.178 | CF₃ | CH₂OCH₂CH₂ | 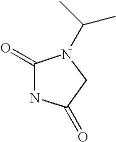 | |
| A1.179 | CF₂Cl | CH₂OCH₂CH₂ | 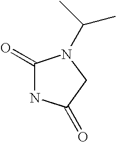 | |
| A1.180 | CHF₂ | CH₂OCH₂CH₂ | 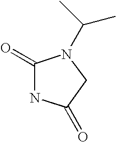 | |
| A1.181 | CF₃ | CH₂ | 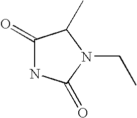 | m.p.: 151° C. |
| A1.182 | CF₂Cl | CH₂ | 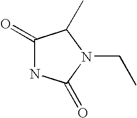 | |
| A1.183 | CHF₂ | CH₂ | 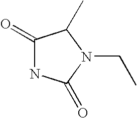 | |
| A1.184 | CF₃ | CH₂OCH₂CH₂ | 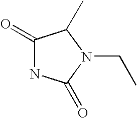 | |
| A1.185 | CF₂Cl | CH₂OCH₂CH₂ | 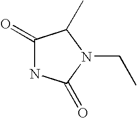 | |
| A1.186 | CHF₂ | CH₂OCH₂CH₂ | 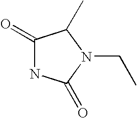 | |
| A1.187 | CF₃ | CH₂ | 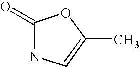 | |
| A1.188 | CF₂Cl | CH₂ | 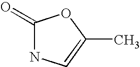 | |
| A1.189 | CHF₂ | CH₂ | 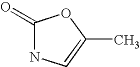 | |
| A1.190 | CF₃ | CH₂OCH₂CH₂ | 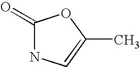 | |
| A1.191 | CF₂Cl | CH₂OCH₂CH₂ | 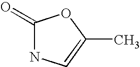 | |
| A1.192 | CHF₂ | CH₂OCH₂CH₂ | 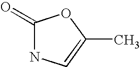 | |
| A1.193 | CF₃ | CH₂ | 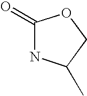 | solid |
| A1.194 | CF₂Cl | CH₂ | 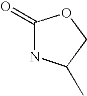 | |
| A1.195 | CHF₂ | CH₂ | 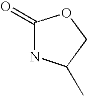 | |
| A1.196 | CF₃ | CH₂OCH₂CH₂ | 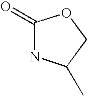 | |
| A1.197 | CF₂Cl | CH₂OCH₂CH₂ | 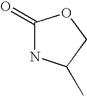 | |
| A1.198 | CHF₂ | CH₂OCH₂CH₂ | 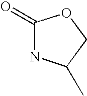 | |
| A1.199 | CF₃ | CH₂ | 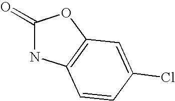 | solid |
| A1.200 | CF₂Cl | CH₂ | 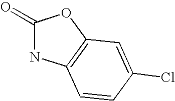 | |
| A1.201 | CHF₂ | CH₂ | 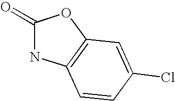 | |
| A1.202 | CF₃ | CH₂ | 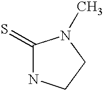 | solid |
| A1.203 | CF₂Cl | CH₂ | 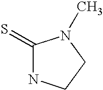 | |
| A1.204 | CHF₂ | CH₂ | 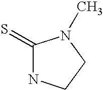 | |
| A1.205 | CF₃ | CH₂OCH₂CH₂ | 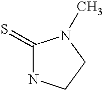 | |
| A1.206 | CF₂Cl | CH₂OCH₂CH₂ | 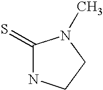 | |
| A1.207 | CHF₂ | CH₂OCH₂CH₂ | 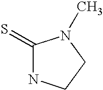 | |
| A1.208 | CF₃ | CH₂ | 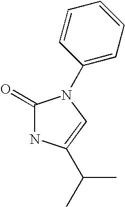 | resin |
| A1.209 | CF₃ | CH₂ | 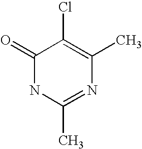 | resin |
| A1.210 | CHF₂ | CH₂ | 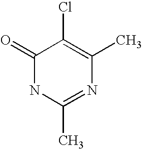 | |
| A1.211 | CF₃ | CH₂ | 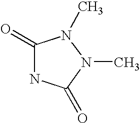 | solid |
| A1.212 | CHF₂ | CH₂ | 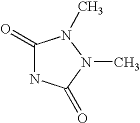 | |
| A1.213 | CF₃ | CH₂ | 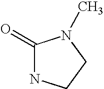 | solid |
| A1.214 | CF₂Cl | CH₂ | 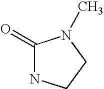 | |
| A1.215 | CF₃ | CH₂ | 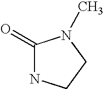 | |
| A1.216 | CF₃ | CH₂OCH₂CH₂ | 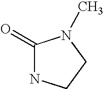 | |
| A1.217 | CF₂Cl | CH₂OCH₂CH₂ | 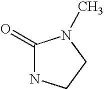 | |
| A1.218 | CHF₂ | CH₂OCH₂CH₂ | 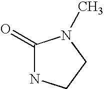 | |
| A1.219 | CF₃ | CH₂ | 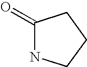 | solid |
| A1.220 | CF₃ | CH₂OCH₂CH₂ | 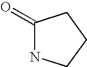 | resin |
| A1.221 | CF₃ | CH₂ | 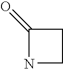 | resin |
| A1.222 | CF₃ | CH₂ | 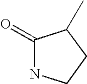 | solid |
| A1.223 | CF₃ | CH₂ | 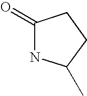 | solid |
| A1.224 | CF₃ | CH₂ | 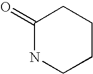 | |
| A1.225 | CClF₂ | CH₂ | 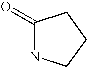 | |
| A1.226 | CClF₂ | CH₂ | 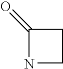 | |
| A1.227 | CClF₂ | CH₂ | 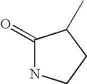 | |
| A1.228 | CClF₂ | CH₂ | 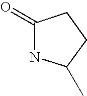 | |
| A1.229 | CClF₂ | CH₂ | 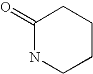 | |
| A1.230 | CHF₂ | CH₂ | 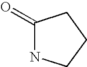 | |
| A1.231 | CHF₂ | CH₂ | 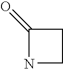 | |
| A1.232 | CHF₂ | CH₂ | 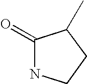 | |
| A1.233 | CHF₂ | CH₂ | 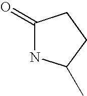 | |
| A1.234 | CHF₂ | CH₂ | 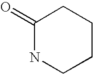 | |
| A1.235 | CF₃ | CH₂ | 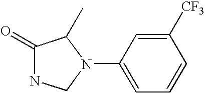 | m.p.: 181° C. |
| A1.236 | CHF₂ | CH₂ | 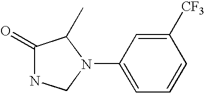 | |
| A1.237 | CF₃ | CH₂ | 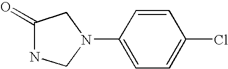 | m.p.: 182° C. |
| A1.238 | CHF₂ | CH₂ | 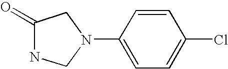 | |
| A1.240 | CF₃ | CH₂ | 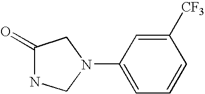 | m.p.: 157° C. |
| A1.241 | CHF₂ | CH₂ | 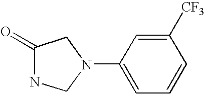 | |
| A1.242 | CF₃ | CH₂ | 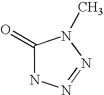 | |
| A1.243 | CF₃ | CH₂ | 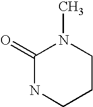 | |
| A1.244 | CF₃ | CH₂ | 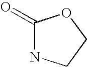 | |
| A1.245 | CF₃ | CH₂ | 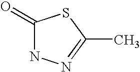 | resin; p = 1 (N-oxide) |

TABLE A2
Compounds of formula IAa₂:

| | |
|---|---|
| | (IAa₂) |
| 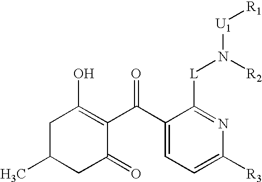 | |

| Comp. No. | R₃ | L | 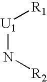 | Phys. data |
|---|---|---|---|---|
| A2.001 | CF₃ | CH₂ | 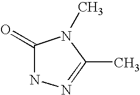 | |
| A2.002 | CF₂H | CH₂ | 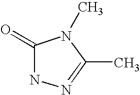 | |
| A2.003 | CF₃ | CH₂ | 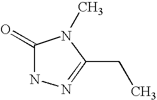 | |
| A2.004 | CF₃ | CH₂ | 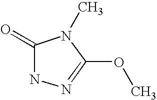 | |
| A2.005 | CF₃ | CH₂ | 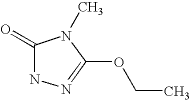 | |
| A2.006 | CF₃ | CH₂ | 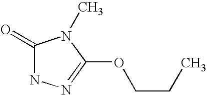 | |
| A2.007 | CF₃ | CH₂ | 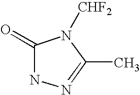 | |
| A2.018 | CF₃ | CH₂ | 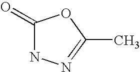 | |
| A2.019 | CF₃ | CH₂ | 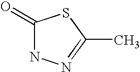 | |
| A2.010 | CF₃ | CH₂ | 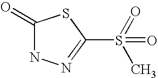 | |
| A2.011 | CF₃ | CH₂ | 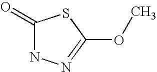 | |
| A2.012 | CF₃ | CH₂ | 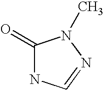 | |
| A2.013 | CF₃ | CH₂ | 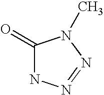 | |
| A2.014 | CF₃ | CH₂ | 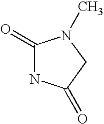 | |
| A2.015 | CF₃ | CH₂ | 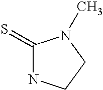 | |
| A2.016 | CF₃ | CH₂ | 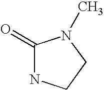 | |
| A2.017 | CF₃ | CH₂ | 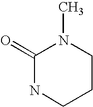 | |
| A2.018 | CF₃ | CH₂ | 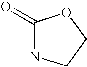 | |
| A2.019 | CF₃ | CH₂ | 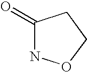 | |
| A2.020 | CF₃ | CH₂ | 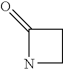 | |
| A2.021 | CF₃ | CH₂ | 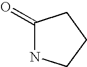 | |
| A2.022 | CF₃ | CH₂ | 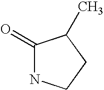 | |
| A2.023 | CF₃ | CH₂ | 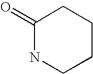 | |

TABLE A3
Compounds of formula IAa₃:

| | | | | (IAa₃) |
|---|---|---|---|---|
| 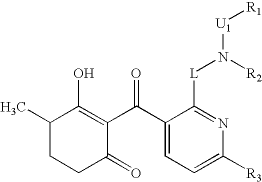 | | | | |
| Comp. No. | R₃ | L | 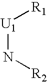 | Phys. data |
| A3.001 | CF₃ | CH₂ | 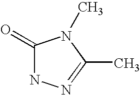 | |
| A3.002 | CF₂H | CH₂ | 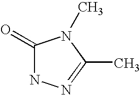 | |
| A3.003 | CF₃ | CH₂ | 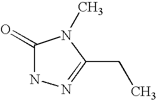 | |
| A3.004 | CF₃ | CH₂ | 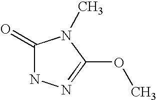 | |
| A3.005 | CF₃ | CH₂ | 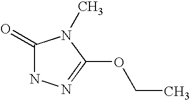 | |
| A3.006 | CF₃ | CH₂ | 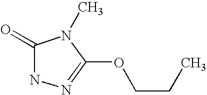 | |
| A3.007 | CF₃ | CH₂ | 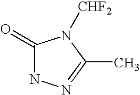 | |
| A3.008 | CF₃ | CH₂ | 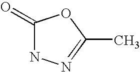 | |
| A3.009 | CF₃ | CH₂ | 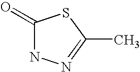 | |
| A3.010 | CF₃ | CH₂ | 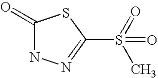 | |
| A3.011 | CF₃ | CH₂ | 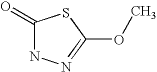 | |
| A3.012 | CF₃ | CH₂ | 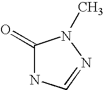 | |
| A3.013 | CF₃ | CH₂ | 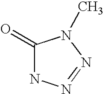 | |
| A3.014 | CF₃ | CH₂ | 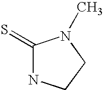 | |
| A3.015 | CF₃ | CH₂ | 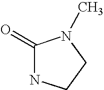 | |
| A3.016 | CF₃ | CH₂ | 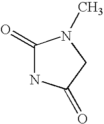 | |
| A3.017 | CF₃ | CH₂ | 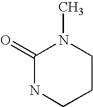 | |
| A3.018 | CF₃ | CH₂ | 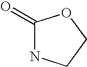 | |
| A3.019 | CF₃ | CH₂ | 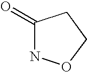 | |
| A3.020 | CF₃ | CH₂ | 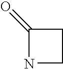 | |
| A3.021 | CF₃ | CH₂ | 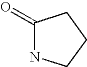 | |
| A3.022 | CF₃ | CH₂ | 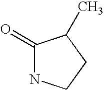 | |
| A3.023 | CF₃ | CH₂ | 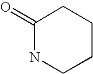 | |

TABLE A4
Compounds of formula IAa₄:

| | | | | (IAa₄) |
|---|---|---|---|---|
| 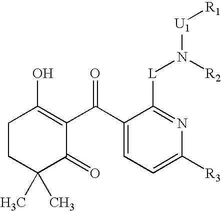 | | | | |
| Comp. No. | R₃ | L | 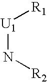 | Phys. data |
| A4.001 | CF₃ | CH₂ | 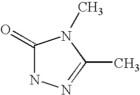 | |
| A4.002 | CF₂H | CH₂ | 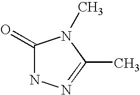 | |
| A4.003 | CF₃ | CH₂ | 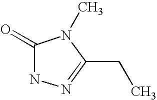 | |
| A4.004 | CF₃ | CH₂ | 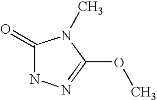 | |
| A4.005 | CF₃ | CH₂ | 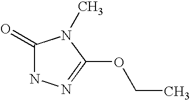 | |
| A4.006 | CF₃ | CH₂ | 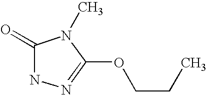 | |
| A4.007 | CF₃ | CH₂ | 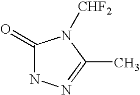 | |
| A4.008 | CF₃ | CH₂ | 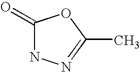 | |
| A4.009 | CF₃ | CH₂ | 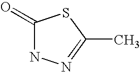 | |
| A4.010 | CF₃ | CH₂ | 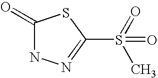 | |
| A4.011 | CF₃ | CH₂ | 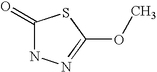 | |
| A4.012 | CF₃ | CH₂ | 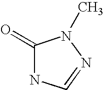 | |
| A4.013 | CF₃ | CH₂ | 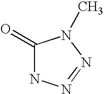 | |
| A4.014 | CF₃ | CH₂ | 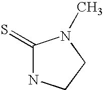 | |
| A4.015 | CF₃ | CH₂ | 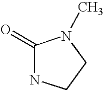 | |
| A4.016 | CF₃ | CH₂ | 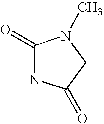 | |
| A4.017 | CF₃ | CH₂ | 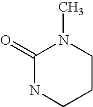 | |
| A4.018 | CF₃ | CH₂ | 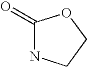 | |
| A4.019 | CF₃ | CH₂ | 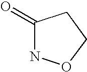 | |
| A4.020 | CF₃ | CH₂ | 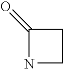 | |
| A4.021 | CF₃ | CH₂ | 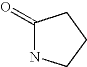 | |
| A4.022 | CF₃ | CH₂ | 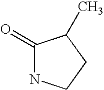 | |
| A4.023 | CF₃ | CH₂ | 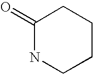 | |

TABLE A5
Compounds of formula IAa₅:

| | | | | (IAa₅) |
|---|---|---|---|---|
| 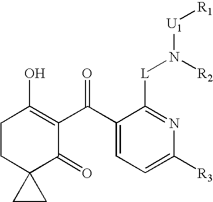 | | | | |
| Comp. No. | R₃ | L | 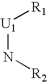 | Phys. data |
| A5.001 | CF₃ | CH₂ | 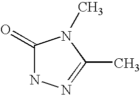 | |
| A5.002 | CF₂H | CH₂ | 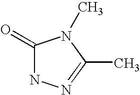 | |
| A5.003 | CF₃ | CH₂ | 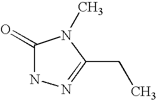 | |
| A5.004 | CF₃ | CH₂ | 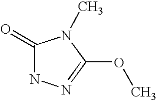 | |
| A5.005 | CF₃ | CH₂ | 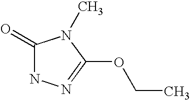 | |
| A5.006 | CF₃ | CH₂ | 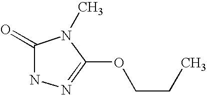 | |
| A5.007 | CF₃ | CH₂ | 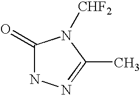 | |
| A5.008 | CF₃ | CH₂ | 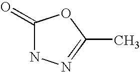 | |
| A5.009 | CF₃ | CH₂ | 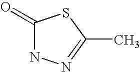 | |
| A5.010 | CF₃ | CH₂ | 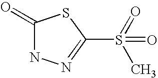 | |
| A5.011 | CF₃ | CH₂ | 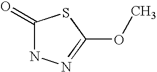 | |
| A5.012 | CF₃ | CH₂ | 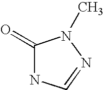 | |
| A5.013 | CF₃ | CH₂ | 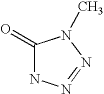 | |
| A5.014 | CF₃ | CH₂ | 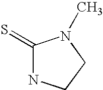 | |
| A5.015 | CF₃ | CH₂ | 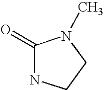 | |
| A5.016 | CF₃ | CH₂ | 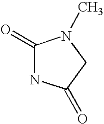 | |
| A5.017 | CF₃ | CH₂ | 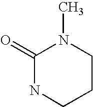 | |
| A5.018 | CF₃ | CH₂ | 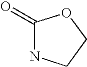 | |
| A5.019 | CF₃ | CH₂ | 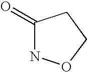 | |
| A5.020 | CF₃ | CH₂ | 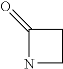 | |
| A5.021 | CF₃ | CH₂ | 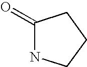 | |
| A5.022 | CF₃ | CH₂ | 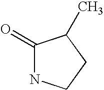 | |
| A5.023 | CF₃ | CH₂ | 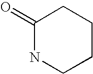 | |

TABLE A6
Compounds of formula IAa₆:

| | |
|---|---|
| | (IAa₆) |
| 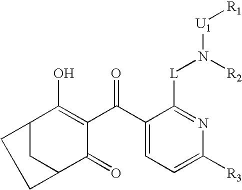 | |

| Comp. No. | R₃ | L | 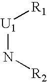 | Phys. data |
|---|---|---|---|---|
| A6.001 | CF₃ | CH₂ | 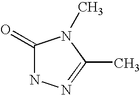 | |
| A6.002 | CF₂H | CH₂ | 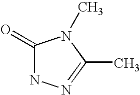 | |
| A6.003 | CF₃ | CH₂ | 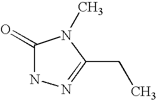 | |
| A6.004 | CF₃ | CH₂OCH₂CH₂ | 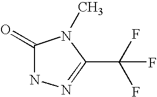 | |
| A6.005 | CF₂Cl | CH₂OCH₂CH₂ | 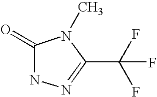 | |
| A6.006 | CHF₂ | CH₂OCH₂CH₂ | 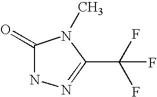 | |
| A6.007 | CF₃ | CH₂ | 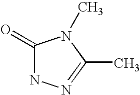 | |
| A6.008 | CF₂Cl | CH₂ | 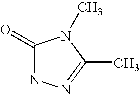 | |
| A6.009 | CHF₂ | CH₂ | 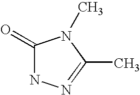 | |
| A6.010 | CF₃ | CH₂OCH₂CH₂ | 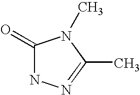 | |
| A6.011 | CF₂Cl | CH₂OCH₂CH₂ | 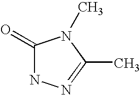 | |
| A6.012 | CHF₂ | CH₂OCH₂CH₂ | 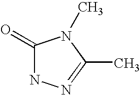 | |
| A6.013 | CF₃ | CH₂ | 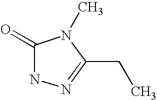 | |
| A6.014 | CF₂Cl | CH₂ | 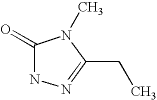 | |
| A6.015 | CHF₂ | CH₂ | 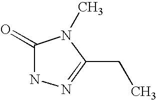 | |
| A6.016 | CF₃ | CH₂OCH₂CH₂ | 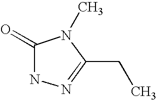 | |
| A6.017 | CF₂Cl | CH₂OCH₂CH₂ | 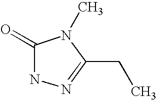 | |
| A6.018 | CHF₂ | CH₂OCH₂CH₂ | 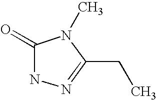 | |
| A6.019 | CF₃ | CH₂ | 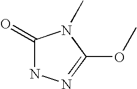 | |
| A6.020 | CF₂Cl | CH₂ | 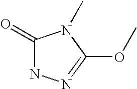 | |
| A6.021 | CHF₂ | CH₂ | 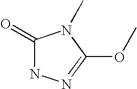 | |
| A6.022 | CF₃ | CH₂OCH₂CH₂ | 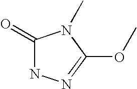 | |
| A6.023 | CF₂Cl | CH₂OCH₂CH₂ | 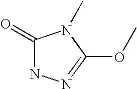 | |
| A6.024 | CHF₂ | CH₂OCH₂CH₂ | 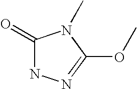 | |
| A6.025 | CF₃ | CH₂ | 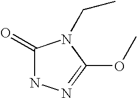 | |
| A6.026 | CF₂Cl | CH₂ | 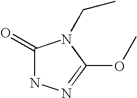 | |
| A6.027 | CHF₂ | CH₂ | 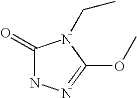 | |
| A6.028 | CF₃ | CH₂OCH₂CH₂ | 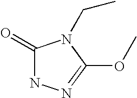 | |
| A6.029 | CF₂Cl | CH₂OCH₂CH₂ | 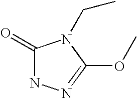 | |
| A6.030 | CHF₂ | CH₂OCH₂CH₂ | 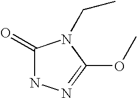 | |
| A6.031 | CF₃ | CH₂ | 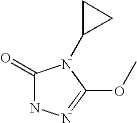 | |
| A6.032 | CF₂Cl | CH₂ | 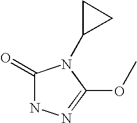 | |
| A6.033 | CHF₂ | CH₂ | 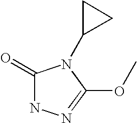 | |
| A6.034 | CF₃ | CH₂OCH₂CH₂ | 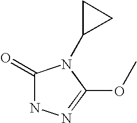 | |
| A6.035 | CF₂Cl | CH₂OCH₂CH₂ | 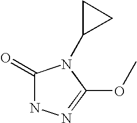 | |
| A6.036 | CHF₂ | CH₂OCH₂CH₂ | 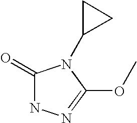 | |
| A6.037 | CF₃ | CH₂ | 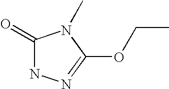 | |
| A6.038 | CF₂Cl | CH₂ | 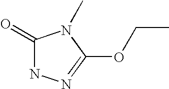 | |
| A6.039 | CHF₂ | CH₂ | 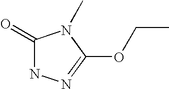 | |
| A6.040 | CF₃ | CH₂OCH₂CH₂ | 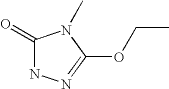 | |
| A6.041 | CF₂Cl | CH₂OCH₂CH₂ | 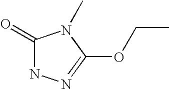 | |
| A6.042 | CHF₂ | CH₂OCH₂CH₂ | 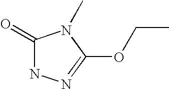 | |
| A6.043 | CF₃ | CH₂ | 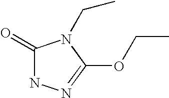 | |
| A6.044 | CF₂Cl | CH₂ | 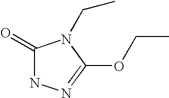 | |
| A6.045 | CHF₂ | CH₂ | 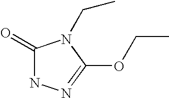 | |
| A6.046 | CF₃ | CH₂OCH₂CH₂ | 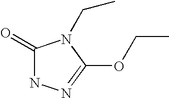 | |
| A6.047 | CF₂Cl | CH₂OCH₂CH₂ | 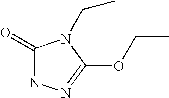 | |
| A6.048 | CHF₂ | CH₂OCH₂CH₂ | 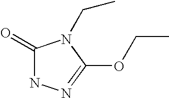 | |
| A6.049 | CF₃ | CH₂ | 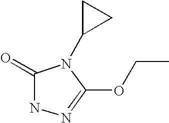 | |
| A6.050 | CF₂Cl | CH₂ | 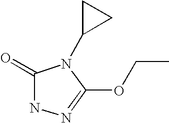 | |
| A6.051 | CHF₂ | CH₂ | 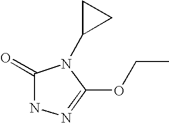 | |
| A6.052 | CF₃ | CH₂OCH₂CH₂ | 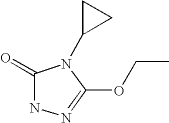 | |
| A6.053 | CF₂Cl | CH₂OCH₂CH₂ | 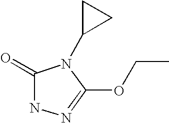 | |
| A6.054 | CHF₂ | CH₂OCH₂CH₂ | 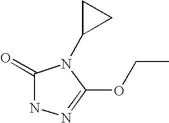 | |
| A6.055 | CF₃ | CH₂ | 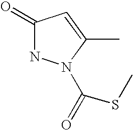 | resin |
| A6.056 | CF₂Cl | CH₂ | 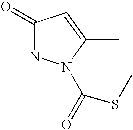 | |
| A6.057 | CHF₂ | CH₂ | 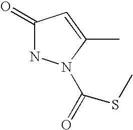 | |
| A6.058 | CF₃ | CH₂OCH₂CH₂ | 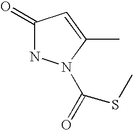 | |
| A6.059 | CF₂Cl | CH₂OCH₂CH₂ | 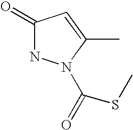 | |
| A6.060 | CHF₂ | CH₂OCH₂CH₂ | 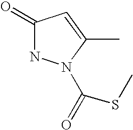 | |
| A6.061 | CF₃ | CH₂ | 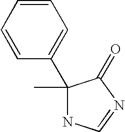 | |
| A6.062 | CF₂Cl | CH₂ | 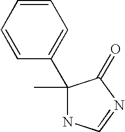 | |
| A6.063 | CHF₂ | CH₂ | 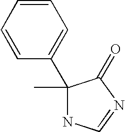 | |
| A6.064 | CF₃ | CH₂OCH₂CH₂ | 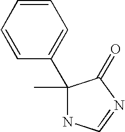 | |
| A6.065 | CF₂Cl | CH₂OCH₂CH₂ | 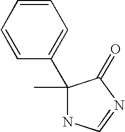 | |
| A6.066 | CHF₂ | CH₂OCH₂CH₂ | 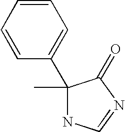 | |
| A6.067 | CF₃ | CH₂ | 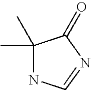 | |
| A6.068 | CF₂Cl | CH₂ | 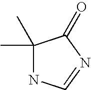 | |
| A6.069 | CHF₂ | CH₂ | 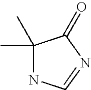 | |
| A6.070 | CF₃ | CH₂OCH₂CH₂ | 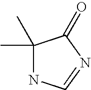 | |
| A6.071 | CF₂Cl | CH₂OCH₂CH₂ | 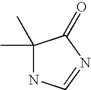 | |
| A6.072 | CHF₂ | CH₂OCH₂CH₂ | 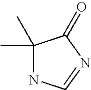 | |
| A6.073 | CF₃ | CH₂ | 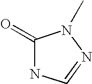 | resin |
| A6.074 | CF₂Cl | CH₂ | 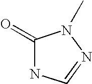 | |
| A6.075 | CHF₂ | CH₂ | 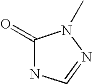 | |
| A6.076 | CF₃ | CH₂OCH₂CH₂ | 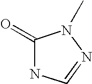 | |
| A6.077 | CF₂Cl | CH₂OCH₂CH₂ | 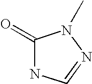 | |
| A6.078 | CHF₂ | CH₂OCH₂CH₂ | 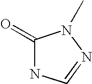 | |
| A6.079 | CF₃ | CH₂ | 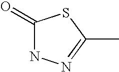 | amorphous crystals |
| A6.080 | CF₂Cl | CH₂ | 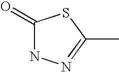 | |
| A6.081 | CHF₂ | CH₂ | 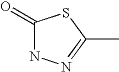 | |
| A6.082 (P3) | CF₃ | CH₂OCH₂CH₂ | 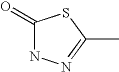 | resin |
| A6.083 | CF₂Cl | CH₂OCH₂CH₂ | 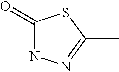 | |
| A6.084 | CHF₂ | CH₂OCH₂CH₂ | 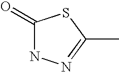 | |
| A6.085 | CF₃ | CH₂ | 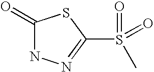 | amorphous crystals |
| A6.086 | CF₂Cl | CH₂ | 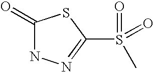 | |
| A6.087 | CHF₂ | CH₂ | 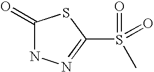 | |
| A6.088 | CF₃ | CH2QCH₂CH₂ | 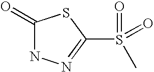 | |
| A6.089 | CF₂Cl | CH₂OCH₂CH₂ | 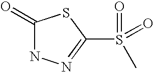 | |
| A6.090 | CHF₂ | CH₂OCH₂CH₂ | 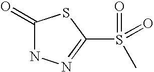 | |
| A6.091 | CF₃ | CH₂ | 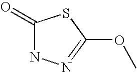 | resin |
| A6.092 | CF₂Cl | CH₂ | 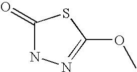 | |
| A6.093 | CHF₂ | CH₂ | 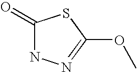 | |
| A6.094 | CF₃ | CH₂OCH₂CH₂ | 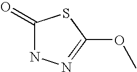 | |
| A6.095 | CF₂Cl | CH₂OCH₂CH₂ | 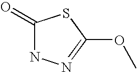 | |
| A6.096 | CHF₂ | CH₂OCH₂CH₂ | 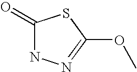 | |
| A6.097 | CF₃ | CH₂ | 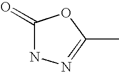 | amorphous crystals |
| A6.098 | CF₂Cl | CH₂ | 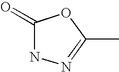 | |
| A6.099 | CHF₂ | CH₂ | 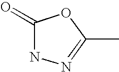 | |
| A6.100 | CF₃ | CH₂OCH₂CH₂ | 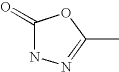 | resin |
| A6.101 | CF₂Cl | CH₂OCH₂CH₂ | 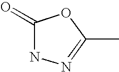 | |
| A6.102 | CHF₂ | CH₂OCH₂CH₂ | 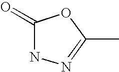 | |
| A6.103 | CF₃ | CH₂ | 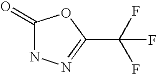 | |
| A6.104 | CF₂Cl | CH₂ | 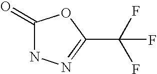 | |
| A6.105 | CHF₂ | CH₂ | 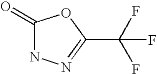 | |
| A6.106 | CF₃ | CH₂OCH₂CH₂ | 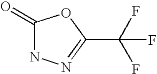 | |
| A6.107 | CF₂Cl | CH₂OCH₂CH₂ | 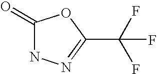 | |
| A6.108 | CHF₂ | CH₂OCH₂CH₂ | 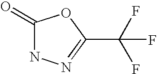 | |
| A6.109 | CF₃ | CH₂ | 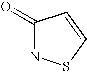 | |
| A6.110 | CF₂Cl | CH₂ | 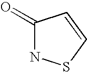 | |
| A6.111 | CHF₂ | CH₂ | 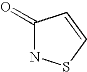 | |
| A6.112 | CF₃ | CH₂OCH₂CH₂ | 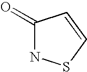 | |
| A6.113 | CF₂Cl | CH₂OCH₂CH₂ | 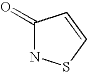 | |
| A6.114 | CHF₂ | CH₂OCH₂CH₂ | 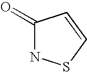 | |
| A6.115 | CF₃ | CH₂ | 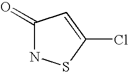 | |
| A6.116 | CF₂Cl | CH₂ | 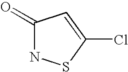 | |
| A6.117 | CHF₂ | CH₂ | 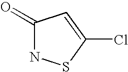 | |
| A6.118 | CF₃ | CH₂OCH₂CH₂ | 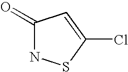 | |
| A6.119 | CF₂Cl | CH₂OCH₂CH₂ | 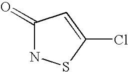 | |
| A6.120 | CHF₂ | CH₂OCH₂CH₂ | 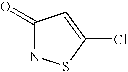 | |
| A6.121 | CF₃ | CH₂ | 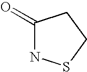 | |
| A6.122 | CF₂Cl | CH₂ | 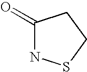 | |
| A6.123 | CHF₂ | CH₂ | 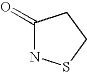 | |
| A6.124 | CF₃ | CH₂OCH₂CH₂ | 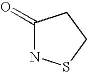 | |
| A6.125 | CF₂Cl | CH₂OCH₂CH₂ | 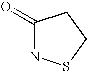 | |
| A6.126 | CHF₂ | CH₂OCH₂CH₂ | 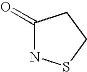 | |
| A6.127 | CF₃ | CH₂ | 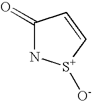 | |
| A6.128 | CF₂Cl | CH₂ | 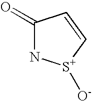 | |
| A6.129 | CHF₂ | CH₂ | 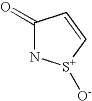 | |
| A6.130 | CF₃ | CH₂OCH₂CH₂ | 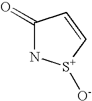 | |
| A6.131 | CF₂Cl | CH₂OCH₂CH₂ | 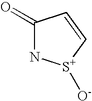 | |
| A6.132 | CHF₂ | CH₂OCH₂CH₂ | 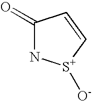 | |
| A6.133 | CF₃ | CH₂ | 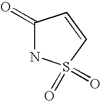 | |
| A6.134 | CF₂Cl | CH₂ | 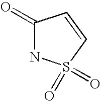 | |
| A6.135 | CHF₂ | CH₂ | 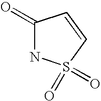 | |
| A6.136 | CF₃ | CH₂OCH₂CH₂ | 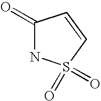 | |
| A6.137 | CF₂Cl | CH₂OCH₂CH₂ | 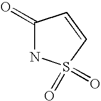 | |
| A6.138 | CHF₂ | CH₂OCH₂CH₂ | 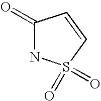 | |
| A6.139 | CF₃ | CH₂ | 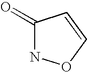 | |
| A6.140 | CF₂Cl | CH₂ | 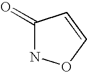 | |
| A6.141 | CHF₂ | CH₂ | 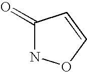 | |
| A6.142 | CF₃ | CH₂OCH₂CH₂ | 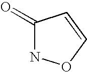 | |
| A6.143 | CF₂Cl | CH₂OCH₂CH₂ | 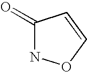 | |
| A6.144 | CHF₂ | CH₂OCH₂CH₂ | 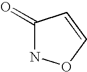 | |
| A6.145 | CF₃ | CH₂ | 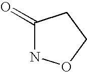 | |
| A6.146 | CF₂Cl | CH₂ | 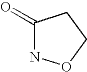 | |
| A6.147 | CHF₂ | CH₂ | 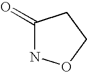 | |
| A6.148 | CF₃ | CH₂OCH₂CH₂ | 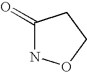 | |
| A6.149 | CF₂Cl | CH₂OCH₂CH₂ | 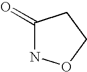 | |
| A6.150 | CHF₂ | CH₂OCH₂CH₂ | 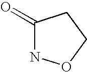 | |
| A6.151 | CF₃ | CH₂ | 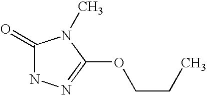 | |
| A6.152 | CF₂Cl | CH₂ | 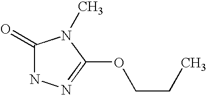 | |
| A6.153 | CHF₂ | CH₂ | 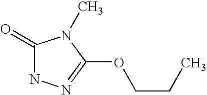 | |
| A6.154 | CF₃ | CH₂OCH₂CH₂ | ~ | 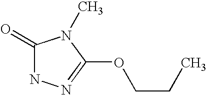 |
| A6.155 | CF₂Cl | CH₂OCH₂CH₂ | 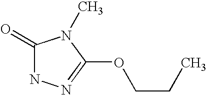 | |
| A6.156 | CHF₂ | CH₂OCH₂CH₂ | 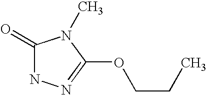 | |
| A6.157 | CF₃ | CH₂ | 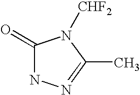 | |
| A6.158 | CF₂Cl | CH₂ | 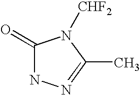 | |
| A6.159 | CHF₂ | CH₂ | 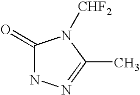 | |
| A6.160 | CF₃ | CH₂OCH₂CH₂ | 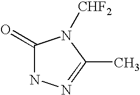 | |
| A6.161 | CF₂Cl | CH₂OCH₂CH₂ | 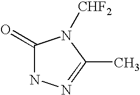 | |
| A6.162 | CHF₂ | CH₂OCH₂CH₂ | 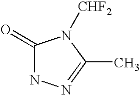 | |
| A6.163 | CF₃ | CH₂ | 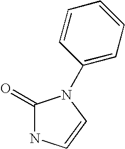 | |
| A6.164 | CF₂Cl | CH₂ | 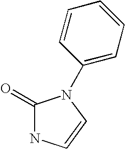 | |
| A6.165 | CHF₂ | CH₂ | 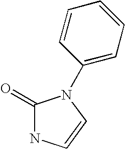 | |
| A6.166 | CF₃ | CH₂OCH₂CH₂ | 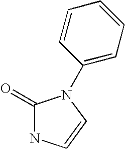 | |
| A6.167 | CF₂Cl | CH₂OCH₂CH₂ | 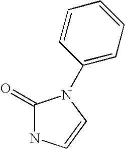 | |
| A6.168 | CHF₂ | CH₂OCH₂CH₂ | 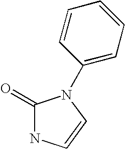 | |
| A6.169 | CF₃ | CH₂ | 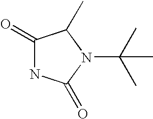 | |
| A6.170 | CF₂Cl | CH₂ | 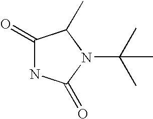 | |
| A6.171 | CHF₂ | CH₂ | 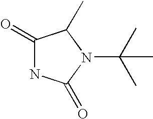 | |
| A6.172 | CF₃ | CH₂OCH₂CH₂ | 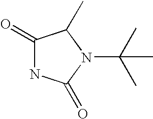 | |
| A6.173 | CF₂Cl | CH₂OCH₂CH₂ | 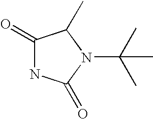 | |
| A6.174 | CHF₂ | CH₂OCH₂CH₂ | 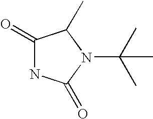 | |
| A6.175 | CF₃ | CH₂ | 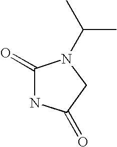 | |
| A6.176 | CF₂Cl | CH₂ | 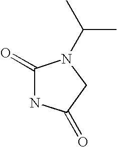 | |
| A6.177 | CHF₂ | CH₂ | 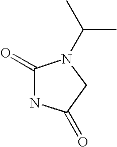 | |
| A6.178 | CF₃ | CH₂OCH₂CH₂ | 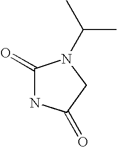 | |
| A6.179 | CF₂Cl | CH₂OCH₂CH₂ | 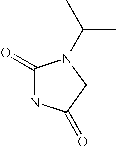 | |
| A6.180 | CHF₂ | CH₂OCH₂CH₂ | 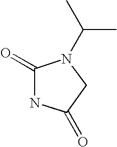 | |
| A6.181 | CF₃ | CH₂ | 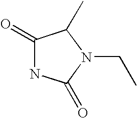 | m.p.: 134° C. |
| A6.182 | CF₂Cl | CH₂ | 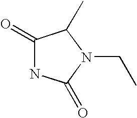 | |
| A6.183 | CHF₂ | CH₂ | 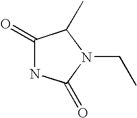 | |
| A6.184 | CF₃ | CH₂OCH₂CH₂ | 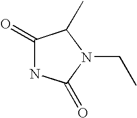 | |
| A6.185 | CF₂Cl | CH₂OCH₂CH₂ | 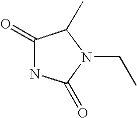 | |
| A6.186 | CHF₂ | CH₂OCH₂CH₂ | 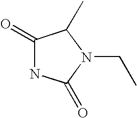 | |
| A6.187 | CF₃ | CH₂ | 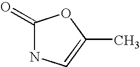 | |
| A6.188 | CF₂Cl | CH₂ | 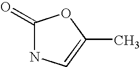 | |
| A6.189 | CHF₂ | CH₂ | 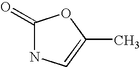 | |
| A6.190 | CF₃ | CH₂OCH₂CH₂ | 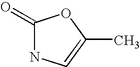 | |
| A6.191 | CF₂Cl | CH₂OCH₂CH₂ | 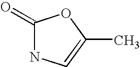 | |
| A6.192 | CHF₂ | CH₂OCH₂CH₂ | 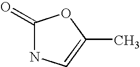 | |
| A6.193 | CF₃ | CH₂ | 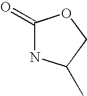 | |
| A6.194 | CF₂Cl | CH₂ | 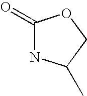 | |
| A6.195 | CHF₂ | CH₂ | 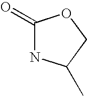 | |
| A6.196 | CF₃ | CH₂OCH₂CH₂ | 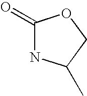 | |
| A6.197 | CF₂Cl | CH₂OCH₂CH₂ | 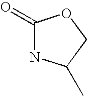 | |
| A6.198 | CHF₂ | CH₂OCH₂CH₂ | 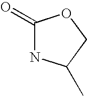 | |
| A6.199 | CF₃ | CH₂ | 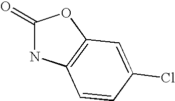 | |
| A6.200 | CF₂Cl | CH₂ | 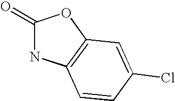 | |
| A6.201 | CHF₂ | CH₂ | 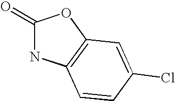 | |
| A6.202 | CF₃ | CH₂ | 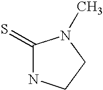 | |
| A6.203 | CF₂Cl | CH₂ | 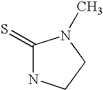 | |
| A6.204 | CHF₂ | CH₂ | 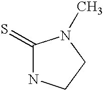 | |
| A6.205 | CF₃ | CH₂OCH₂CH₂ | 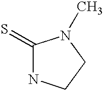 | |
| A6.206 | CF₂Cl | CH₂OCH₂CH₂ | 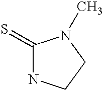 | |
| A6.207 | CHF₂ | CH₂OCH₂CH₂ | 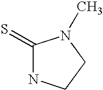 | |
| A6.208 | CF₃ | CH₂ | 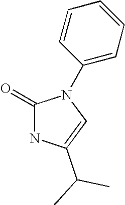 | resin |
| A6.209 | CF₃ | CH₂ | 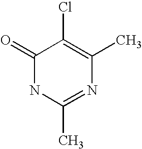 | |
| A6.210 | CHF₂ | CH₂ | 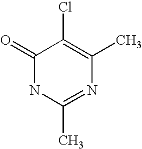 | |
| A6.211 | CF₃ | CH₂ | 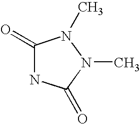 | |
| A6.212 | CHF₂ | CH₂ | 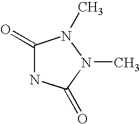 | |
| A6.213 | CF₃ | CH₂ | 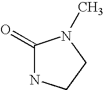 | |
| A6.214 | CF₂Cl | CH₂ | 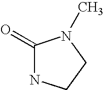 | |
| A6.215 | CHF₂ | CH₂ | 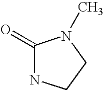 | |
| A6.216 | CF₃ | CH₂OCH₂CH₂ | 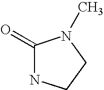 | |
| A6.217 | CF₂Cl | CH₂OCH₂CH₂ | 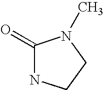 | |
| A6.218 | CHF₂ | CH₂OCH₂CH₂ | 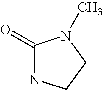 | |
| A6.219 | CH₂ | CF₃ | 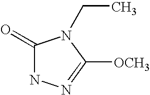 | |
| A6.220 | CH₂ | CF₂Cl | 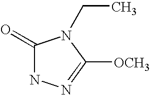 | |
| A6.221 | CH₂ | CHF₂ | 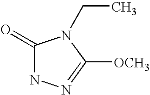 | |
| A6.222 | CH₂OCH₂CH₂ | CF₃ | 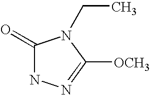 | |
| A6.223 | CH₂OCH₂CH₂ | CF₂Cl | 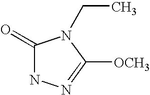 | |
| A6.224 | CH₂OCH₂CH₂ | CHF₂ | 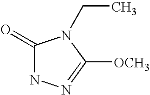 | |
| A6.225 | CF₃ | CH₂ | 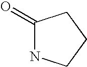 | |
| A6.226 | CF₃ | CH₂OCH₂CH₂ | 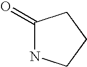 | |
| A6.227 | CF₃ | CH₂ | 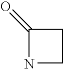 | |
| A6.228 | CF₃ | CH₂ | 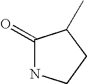 | |
| A6.229 | CF₃ | CH₂ | 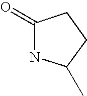 | |
| A6.230 | CClF₂ | CH₂ | 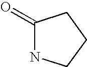 | |
| A6.231 | CClF₂ | CH₂ | 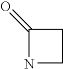 | |
| A6.232 | CClF₂ | | 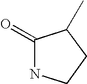 | |
| A6.233 | CClF₂ | | 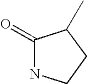 | |
| A6.234 | CHF₂ | | 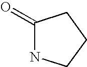 | |
| A6.235 | CHF₂ | | 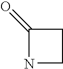 | |
| A6.236 | CHF₂ | | 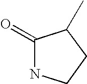 | |
| A6.237 | CHF₂ | | 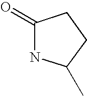 | |
| A6.238 | CF₃ | CH₂ | 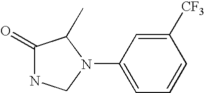 | resin |
| A6.239 | CHF₂ | CH₂ | 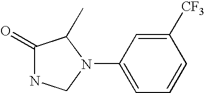 | |
| A6.240 | CF₃ | CH₂ | 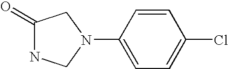 | m.p.: 113° C. |
| A6.241 | CHF₂ | CH₂ | 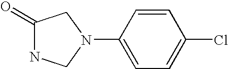 | |
| A6.242 | CF₃ | CH₂ | 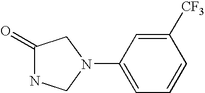 | resin |
| A6.243 | CHF₂ | CH₂ | 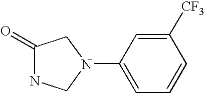 | |
| A6.244 | CF₃ | CH₂ | 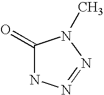 | |
| A6.245 | CF₃ | CH₂ | 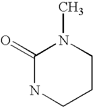 | |
| A6.246 | CF₃ | CH₂ | 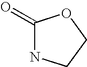 | |
| A6.247 | CF₃ | CH₂OCH₂CH₂ | 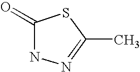 | resin; p = 1 (N-oxide) |

TABLE A7
Compounds of formula IAa₇:

| | |
|---|---|
| | (IAa₇) |
| 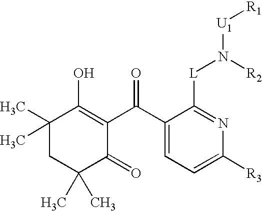 | |

| Comp. No. | R₃ | L | 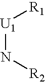 | Phys. data |
|---|---|---|---|---|
| A7.001 | CF₃ | CH₂ | 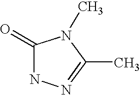 | |
| A7.002 | CF₂H | CH₂ | 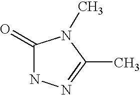 | |
| A7.003 | CF₃ | CH₂ | 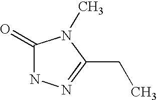 | |
| A7.004 | CF₃ | CH₂ | 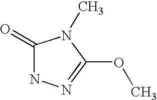 | |
| A7.005 | CF₃ | CH₂ | 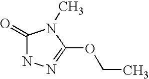 | |
| A7.006 | CF₃ | CH₂ | 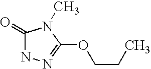 | |
| A7.007 | CF₃ | CH₂ | 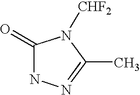 | |
| A7.008 | CF₃ | CH₂ | 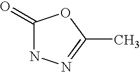 | amorphous crystals |
| A7.009 | CF₃ | CH₂ | 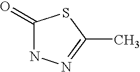 | amorphous crystals |
| A7.010 | CF₃ | CH₂ | 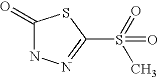 | |
| A7.011 | CF₃ | CH₂OCH₂CH₂ | 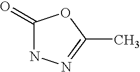 | |
| A7.012 | CF₃ | CH₂OCH₂CH₂ | 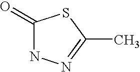 | |
| A7.013 | CF₃ | CH₂ | 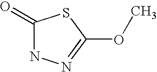 | |
| A7.014 | CF₃ | CH₂ | 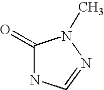 | |
| A7.015 | CF₃ | CH₂ | 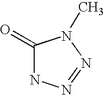 | |
| A7.016 | CF₃ | CH₂ | 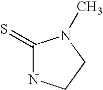 | |
| A7.017 | CF₃ | CH₂ | 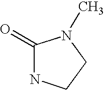 | |
| A7.018 | CF₃ | CH₂ | 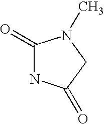 | |
| A7.019 | CF₃ | CH₂ | 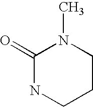 | |
| A7.020 | CF₃ | CH₂ | 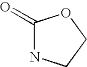 | |
| A7.021 | CF₃ | CH₂ | 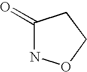 | |
| A7.022 | CF₃ | CH₂ | 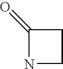 | |
| A7.023 | CF₃ | CH₂ | 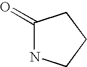 | |
| A7.024 | CF₃ | CH₂ | 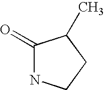 | |
| A7.025 | CF₃ | CH₂ | 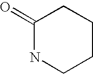 | |
| A7.026 | CF₃ | CH₂ | 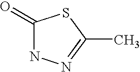 | resin; p = 1 (N-oxide) |

TABLE 8
Compounds of formula IAa₈:

| | |
|---|---|
| | (IAa₈) |
| 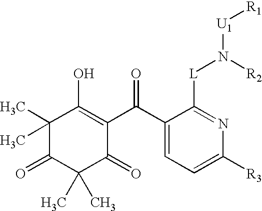 | |

| Comp. No. | R₃ | L | 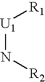 | Phys. data |
|---|---|---|---|---|
| A8.001 | CF₃ | CH₂ | 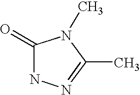 | |
| A8.002 | CF₂H | CH₂ | 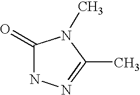 | |
| A8.003 | CF₃ | CH₂ | 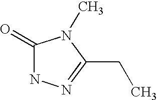 | |
| A8.004 | CF₃ | CH₂ | 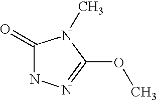 | |
| A8.005 | CF₃ | CH₂ | 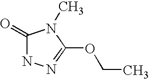 | |
| A8.006 | CF₃ | CH₂ | 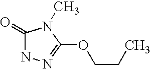 | |
| A8.007 | CF₃ | CH₂ | 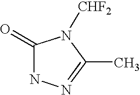 | |
| A8.008 | CF₃ | CH₂ | 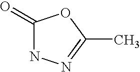 | solid |
| A8.009 | CF₃ | CH₂ | 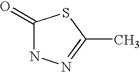 | |
| A8.010 | CF₃ | CH₂ | 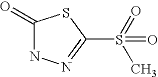 | |
| A8.011 | CF₃ | CH₂OCH₂CH₂ | 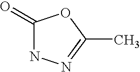 | |
| A8.012 | CF₃ | CH₂OCH₂CH₂ | 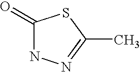 | |
| A8.013 | CF₃ | CH₂ | 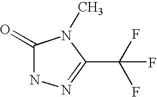 | |
| A8.014 | CF₂Cl | CH₂ | 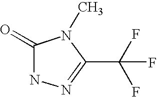 | |
| A8.015 | CF₂H | CH₂ | 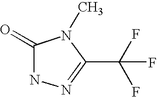 | |
| A8.016 | CF₃ | CH₂OCH₂CH₂ | 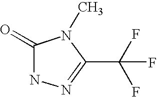 | |
| A8.017 | CF₂Cl | CH₂OCH₂CH₂ | 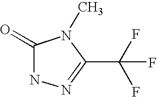 | |
| A8.018 | CHF₂ | CH₂OCH₂CH₂ | 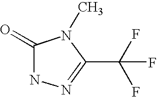 | |
| A8.019 | CF₂Cl | CH₂ | 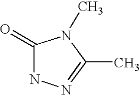 | |
| A8.020 | CF₃ | CH₂OCH₂CH₂ | 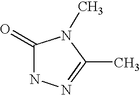 | |
| A8.021 | CF₂Cl | CH₂OCH₂CH₂ | 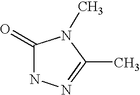 | |
| A8.022 | CHF₂ | CH₂OCH₂CH₂ | 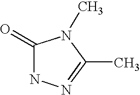 | |
| A8.023 | CF₂Cl | CH₂ | 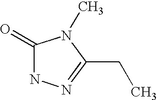 | |
| A8.024 | CHF₂ | CH₂ | 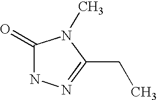 | |
| A8.025 | CF₃ | CH₂OCH₂CH₂ | 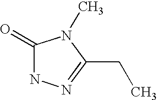 | |
| A8.026 | CF₂Cl | CH₂OCH₂CH₂ | 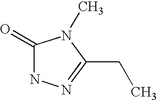 | |
| A8.027 | CHF₂ | CH₂OCH₂CH₂ | 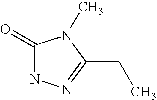 | |
| A8.028 | CF₂Cl | CH₂ | 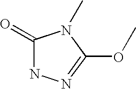 | |
| A8.029 | CHF₂ | CH₂ | 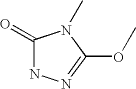 | |
| A8.030 | CF₃ | CH₂OCH₂CH₂ | 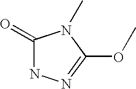 | |
| A8.031 | CF₂Cl | CH₂OCH₂CH₂ | 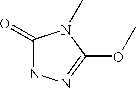 | |
| A8.032 | CHF₂ | CH₂OCH₂CH₂ | 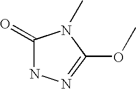 | |
| A8.033 | CF₃ | CH₂ | 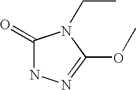 | |
| A8.034 | CF₂Cl | CH₂ | 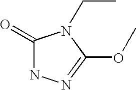 | |
| A8.035 | CHF₂ | CH₂ | 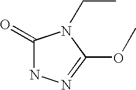 | |
| A8.036 | CF₃ | CH₂OCH₂CH₂ | 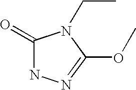 | |
| A8.037 | CF₂Cl | CH₂OCH₂CH₂ | 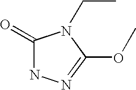 | |
| A8.038 | CHF₂ | CH₂OCH₂CH₂ | 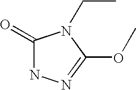 | |
| A8.039 | CF₃ | CH₂ | 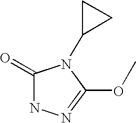 | |
| A8.040 | CF₂Cl | CH₂ | 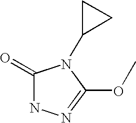 | |
| A8.041 | CHF₂ | CH₂ | 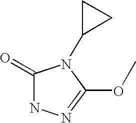 | |
| A8.042 | CF₃ | CH₂OCH₂CH₂ | 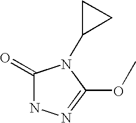 | |
| A8.043 | CF₂Cl | CH₂OCH₂CH₂ | 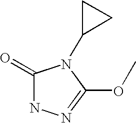 | |
| A8.044 | CHF₂ | CH₂OCH₂CH₂ | 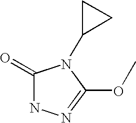 | |
| A8.050 | CF₃ | CH₂ | 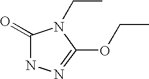 | |
| A8.051 | CF₂Cl | CH₂ | 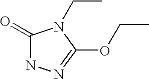 | |
| A8.052 | CHF₂ | CH₂ | 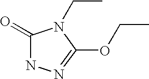 | |
| A8.053 | CF₃ | CH₂OCH₂CH₂ | 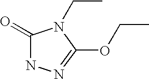 | |
| A8.054 | CF₂Cl | CH₂OCH₂CH₂ | 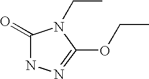 | |
| A8.055 | CHF₂ | CH₂OCH₂CH₂ | 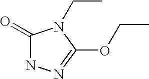 | |
| A8.056 | CF₃ | CH₂ | 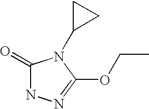 | |
| A8.057 | CF₂Cl | CH₂ | 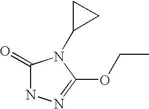 | |
| A8.058 | CHF₂ | CH₂ | 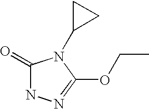 | |
| A8.059 | CF₃ | CH₂OCH₂CH₂ | 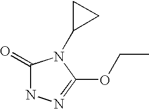 | |
| A8.060 | CF₂Cl | CH₂OCH₂CH₂ | 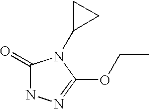 | |
| A8.061 | CHF₂ | CH₂OCH₂CH₂ | 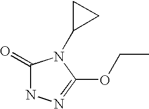 | |
| A8.062 | CF₃ | CH₂ | 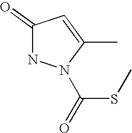 | resin |
| A8.063 | CF₂Cl | CH₂ | 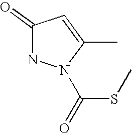 | |
| A8.064 | CHF₂ | CH₂ | 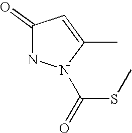 | |
| A8.065 | CF₃ | CH₂OCH₂CH₂ | 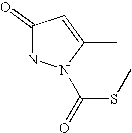 | |
| A8.066 | CF₂Cl | CH₂OCH₂CH₂ | 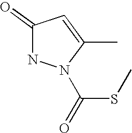 | |
| A8.067 | CHF₂ | CH₂OCH₂CH₂ | 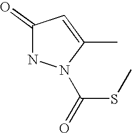 | |
| A8.068 | CF₃ | CH₂ | 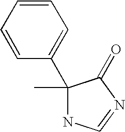 | |
| A8.069 | CF₂Cl | CH₂ | 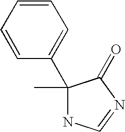 | |
| A8.070 | CHF₂ | CH₂ | 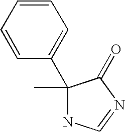 | |
| A8.071 | CF₃ | CH₂OCH₂CH₂ | 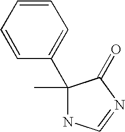 | |
| A8.072 | CF₂Cl | CH₂OCH₂CH₂ | 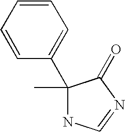 | |
| A8.073 | CHF₂ | CH₂OCH₂CH₂ | 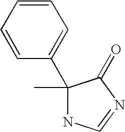 | |
| A8.074 | CF₃ | CH₂ | 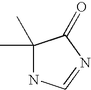 | |
| A8.075 | CF₂Cl | CH₂ | 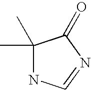 | |
| A8.076 | CHF₂ | CH₂ | 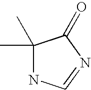 | |
| A8.077 | CF₃ | CH₂OCH₂CH₂ | 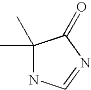 | |
| A8.078 | CF₂Cl | CH₂OCH₂CH₂ | 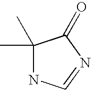 | |
| A8.079 | CHF₂ | CH₂OCH₂CH₂ | 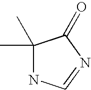 | |
| A8.080 | CF₃ | CH₂ | 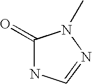 | resin |
| A8.081 | CF₂Cl | CH₂ | 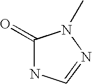 | |
| A8.082 | CHF₂ | CH₂ | 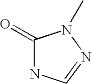 | |
| A8.083 | CF₃ | CH₂OCH₂CH₂ | 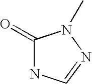 | |
| A8.084 | CF₂Cl | CH₂OCH₂CH₂ | 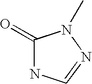 | |
| A8.085 | CHF₂ | CH₂OCH₂CH₂ | 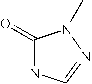 | |
| A8.086 | CF₂Cl | CH₂ | 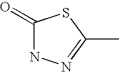 | |
| A8.087 | CHF₂ | CH₂ | 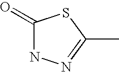 | |
| A8.088 | CF₂Cl | CH₂OCH₂CH₂ | 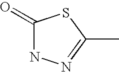 | |
| A8.089 | CHF₂ | CH₂OCH₂CH₂ | 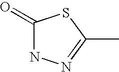 | |
| A8.090 | CF₂Cl | CH₂ | 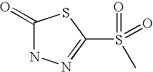 | |
| A8.091 | CHF₂ | CH₂ | 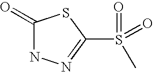 | |
| A8.092 | CF₃ | CH₂OCH₂CH₂ | 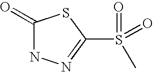 | |
| A8.093 | CF₂Cl | CH₂OCH₂CH₂ | 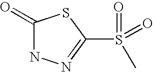 | |
| A8.094 | CHF₂ | CH₂OCH₂CH₂ | 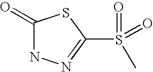 | |
| A8.095 | CF₃ | CH₂ | 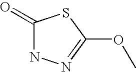 | resin |
| A8.096 | CF₂Cl | CH₂ | 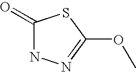 | |
| A8.097 | CHF₂ | CH₂ | 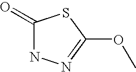 | |
| A8.098 | CF₃ | CH₂OCH₂CH₂ | 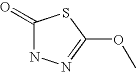 | |
| A8.099 | CF₂Cl | CH₂OCH₂CH₂ | 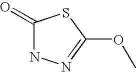 | |
| A8.100 | CHF₂ | CH₂OCH₂CH₂ | 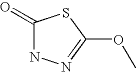 | |
| A8.101 | CF₂Cl | CH₂ | 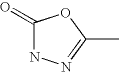 | |
| A8.102 | CHF₂ | CH₂ | 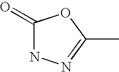 | |
| A8.103 | CF₂Cl | CH₂OCH₂CH₂ | 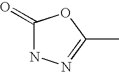 | |
| A8.104 | CHF₂ | CH₂OCH₂CH₂ | 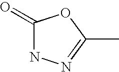 | |
| A8.105 | CF₃ | CH₂ | 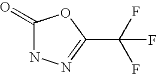 | |
| A8.106 | CF₂Cl | CH₂ | 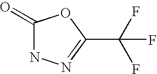 | |
| A8.107 | CHF₂ | CH₂ | 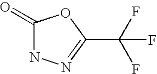 | |
| A8.108 | CF₃ | CH₂OCH₂CH₂ | 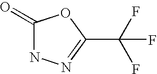 | |
| A8.109 | CF₂Cl | CH₂OCH₂CH₂ | 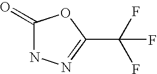 | |
| A8.110 | CHF₂ | CH₂OCH₂CH₂ | 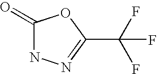 | |
| A8.111 | CF₃ | CH₂ | 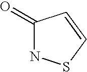 | |
| A8.112 | CF₂Cl | CH₂ | 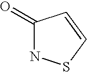 | |
| A8.113 | CHF₂ | CH₂ | 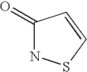 | |
| A8.114 | CF₃ | CH₂OCH₂CH₂ | 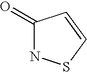 | |
| A8.115 | CF₂Cl | CH₂OCH₂CH₂ | 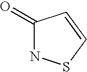 | |
| A8.116 | CHF₂ | CH₂OCH₂CH₂ | 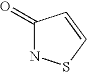 | |
| A8.117 | CF₃ | CH₂ | 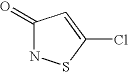 | |
| A8.118 | CF₂Cl | CH₂ | 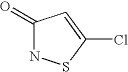 | |
| A8.119 | CHF₂ | CH₂ | 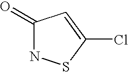 | |
| A8.120 | CF₃ | CH₂OCH₂CH₂ | 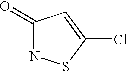 | |
| A8.121 | CF₂Cl | CH₂OCH₂CH₂ | 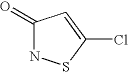 | |
| A8.122 | CHF₂ | CH₂OCH₂CH₂ | 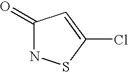 | |
| A8.123 | CF₃ | CH₂ | 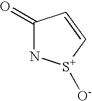 | |
| A8.124 | CF₂Cl | CH₂ | 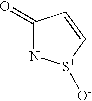 | |
| A8.125 | CHF₂ | CH₂ | 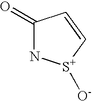 | |
| A8.126 | CF₃ | CH₂OCH₂CH₂ | 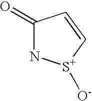 | |
| A8.127 | CF₂Cl | CH₂OCH₂CH₂ | 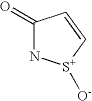 | |
| A8.128 | CHF₂ | CH₂OCH₂CH₂ | 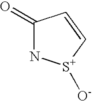 | |
| A8.129 | CF₃ | CH₂ | 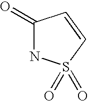 | |
| A8.130 | CF₂Cl | CH₂ | 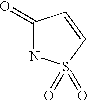 | |
| A8.131 | CHF₂ | CH₂ | 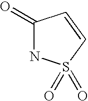 | |
| A8.132 | CF₃ | CH₂OCH₂CH₂ | 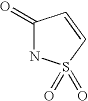 | |
| A8.133 | CF₂Cl | CH₂OCH₂CH₂ | 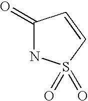 | |
| A8.134 | CHF₂ | CH₂OCH₂CH₂ | 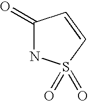 | |
| A8.135 | CF₃ | CH₂ | 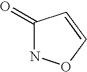 | |
| A8.136 | CF₂Cl | CH₂ | 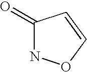 | |
| A8.137 | CHF₂ | CH₂ | 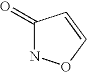 | |
| A8.138 | CF₃ | CH₂OCH₂CH₂ | 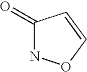 | |
| A8.139 | CF₂Cl | CH₂OCH₂CH₂ | 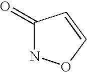 | |
| A8.140 | CHF₂ | CH₂OCH₂CH₂ | 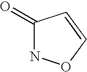 | |
| A8.141 | CF₂Cl | CH₂ | 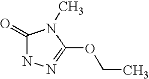 | |
| A8.142 | CHF₂ | CH₂ | 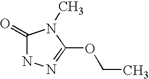 | |
| A8.143 | CF₃ | CH₂OCH₂CH₂ | 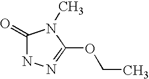 | |
| A8.144 | CF₂Cl | CH₂OCH₂CH₂ | 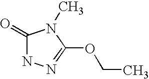 | |
| A8.145 | CHF₂ | CH₂OCH₂CH₂ | 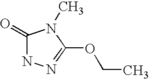 | |
| A8.146 | CF₂Cl | CH₂ | 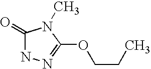 | |
| A8.147 | CHF₂ | CH₂ | 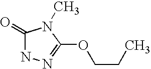 | |
| A8.148 | CF₃ | CH₂OCH₂CH₂ | 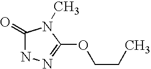 | |
| A8.149 | CF₂Cl | CH₂OCH₂CH₂ | 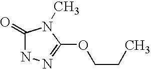 | |
| A8.150 | CHF₂ | CH₂OCH₂CH₂ | 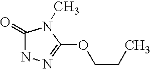 | |
| A8.151 | CF₂Cl | CH₂ | 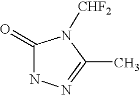 | |
| A8.152 | CHF₂ | CH₂ | 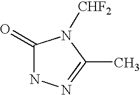 | |
| A8.153 | CF₃ | CH₂OCH₂CH₂ | 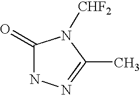 | |
| A8.154 | CF₂Cl | CH₂OCH₂CH₂ | 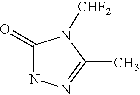 | |
| A8.155 | CHF₂ | CH₂OCH₂CH₂ | 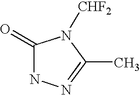 | |
| A8.156 | CF₃ | CH₂ | 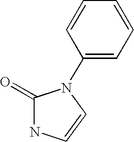 | |
| A8.157 | CF₂Cl | CH₂ | 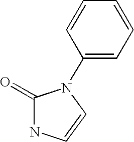 | |
| A8.158 | CHF₂ | CH₂ | 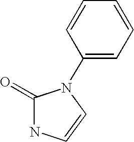 | |
| A8.159 | CF₃ | CH₂OCH₂CH₂ | 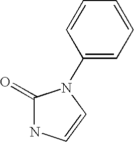 | |
| A8.160 | CF₂Cl | CH₂OCH₂CH₂ | 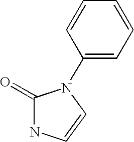 | |
| A8.161 | CHF₂ | CH₂OCH₂CH₂ | 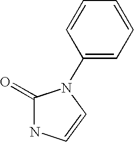 | |
| A8.162 | CF₃ | CH₂ | 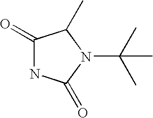 | |
| A8.163 | CF₂Cl | CH₂ | 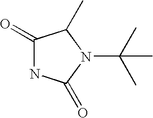 | |
| A8.164 | CHF₂ | CH₂ | 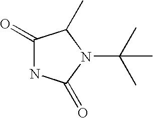 | |
| A8.165 | CF₃ | CH₂OCH₂CH₂ | 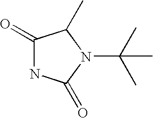 | |
| A8.166 | CF₂Cl | CH₂OCH₂CH₂ | 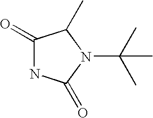 | |
| A8.167 | CHF₂ | CH₂OCH₂CH₂ | 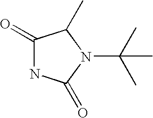 | |
| A8.168 | CF₃ | CH₂ | 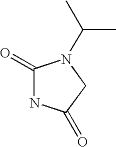 | m.p.: 65° C. |
| A8.169 | CF₂Cl | CH₂ | 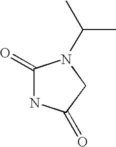 | |
| A8.170 | CHF₂ | CH₂ | 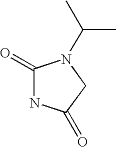 | |
| A8.171 | CF₃ | CH₂OCH₂CH₂ | 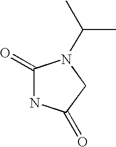 | |
| A8.172 | CF₂Cl | CH₂OCH₂CH₂ | 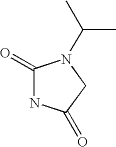 | |
| A8.173 | CHF₂ | CH₂OCH₂CH₂ | 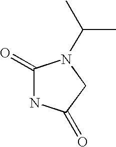 | |
| A8.174 | CF₃ | CH₂ | 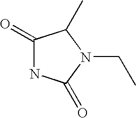 | resin |
| A8.175 | CF₂Cl | CH₂ | 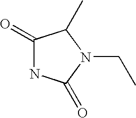 | |
| A8.176 | CHF₂ | CH₂ | 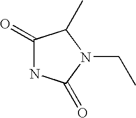 | |
| A8.177 | CF₃ | CH₂OCH₂CH₂ | 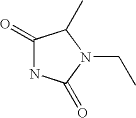 | |
| A8.178 | CF₂Cl | CH₂OCH₂CH₂ | 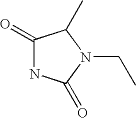 | |
| A8.179 | CHF₂ | CH₂OCH₂CH₂ | 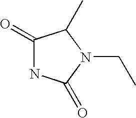 | |
| A8.180 | CF₃ | CH₂ | 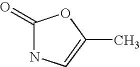 | |
| A8.181 | CF₂Cl | CH₂ | 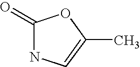 | |
| A8.182 | CHF₂ | CH₂ | 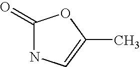 | |
| A8.183 | CF₃ | CH₂OCH₂CH₂ | 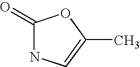 | |
| A8.184 | CF₂Cl | CH₂OCH₂CH₂ | 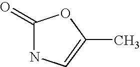 | |
| A8.185 | CHF₂ | CH₂OCH₂CH₂ | 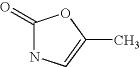 | |
| A8.186 | CF₃ | CH₂ | 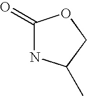 | |
| A8.187 | CF₂Cl | CH₂ | 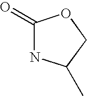 | |
| A8.188 | CHF₂ | CH₂ | 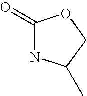 | |
| A8.189 | CF₃ | CH₂OCH₂CH₂ | 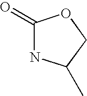 | |
| A8.190 | CF₂Cl | CH₂OCH₂CH₂ | 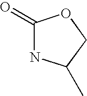 | |
| A8.191 | CHF₂ | CH₂OCH₂CH₂ | 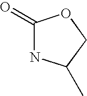 | |
| A8.192 | CF₃ | CH₂ | 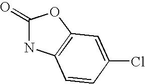 | |
| A8.193 | CF₂Cl | CH₂ | 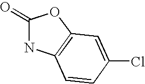 | |
| A8.194 | CHF₂ | CH₂ | 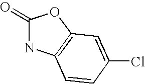 | |
| A8.195 | CF₃ | CH₂ | 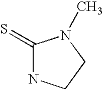 | |
| A8.196 | CF₂Cl | CH₂ | 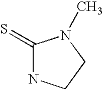 | |
| A8.197 | CHF₂ | CH₂ | 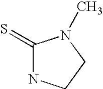 | |
| A8.198 | CF₃ | CH₂OCH₂CH₂ | 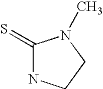 | |
| A8.199 | CF₂Cl | CH₂OCH₂CH₂ | 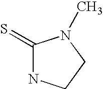 | |
| A8.200 | CHF₂ | CH₂OCH₂CH₂ | 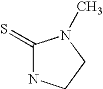 | |
| A8.201 | CF₃ | CH₂ | 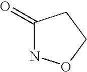 | |
| A8.202 | CF₂Cl | CH₂ | 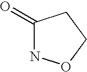 | |
| A8.203 | CHF₂ | CH₂ | 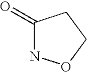 | |
| A8.204 | CF₃ | CH₂ | 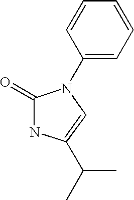 | resin |
| A8.205 | CF₃ | CH₂ | 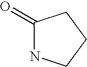 | |
| A8.206 | CF₃ | CH₂OCH₂CH₂ | 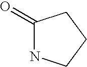 | |
| A8.207 | CF₃ | CH₂ | 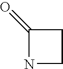 | |
| A8.208 | CF₃ | CH₂ | 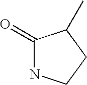 | |
| A8.209 | CF₃ | CH₂ | 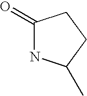 | |
| A8.210 | CClF₂ | CH₂ | 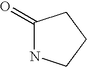 | |
| A8.211 | CClF₂ | CH₂ | 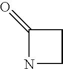 | |
| A8.212 | CClF₂ | CH₂ | 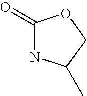 | |
| A8.213 | CClF₂ | CH₂ | 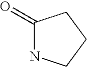 | |
| A8.214 | CHF₂ | CH₂ | 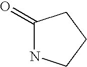 | |
| A8.215 | CHF₂ | CH₂ | 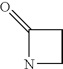 | |
| A8.216 | CHF₂ | CH₂ | 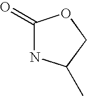 | |
| A8.217 | CHF₂ | CH₂ | 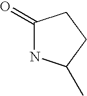 | |
| A8.218 | CF₃ | CH₂ | 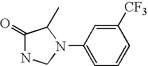 | resin |
| A8.219 | CHF₂ | CH₂ | 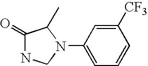 | |
| A8.220 | CF₃ | CH₂ | 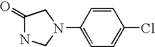 | m.p.: 69° C. |
| A8.221 | CHF₂ | CH₂ | 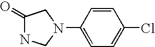 | |
| A8.222 | CF₃ | CH₂ | 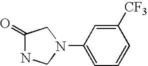 | |
| A8.223 | CHF₂ | CH₂ | 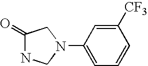 | |
| A8.224 | CF₃ | CH₂ | 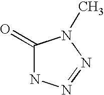 | |
| A8.225 | CF₃ | CH₂ | 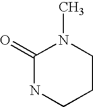 | |
| A8.226 | CF₃ | CH₂ | 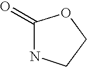 | |

TABLE A9
Compounds of formula IAa₉:

| | |
|---|---|
| | (IAa₉) |
| 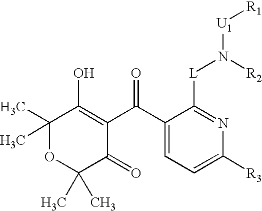 | |

| Comp. No. | R₃ | L | 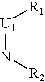 | Phys. data |
|---|---|---|---|---|
| A9.001 | CF₃ | CH₂ | 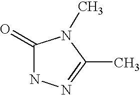 | |
| A9.002 | CF₂H | CH₂ | 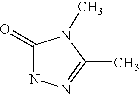 | |
| A9.003 | CF₃ | CH₂ | 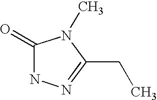 | |
| A9.004 | CF₃ | CH₂ | 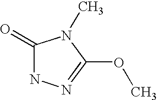 | |
| A9.005 | CF₃ | CH₂ | 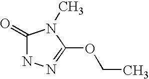 | |
| A9.006 | CF₃ | CH₂ | 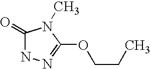 | |
| A9.007 | CF₃ | CH₂ | 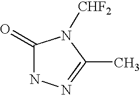 | |
| A9.008 | CF₃ | CH₂ | 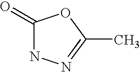 | |
| A9.009 | CF₃ | CH₂ | 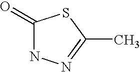 | |
| A9.010 | CF₃ | CH₂ | 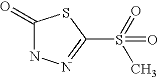 | |
| A9.011 | CF₃ | CH₂ | 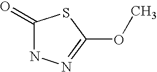 | |
| A9.012 | CF₃ | CH₂ | 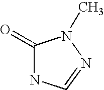 | |
| A9.013 | CF₃ | CH₂ | 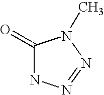 | |
| A9.014 | CF₃ | CH₂ | 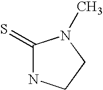 | |
| A9.015 | CF₃ | CH₂ | 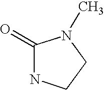 | |
| A9.016 | CF₃ | CH₂ | 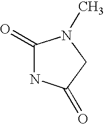 | |
| A9.017 | CF₃ | CH₂ | 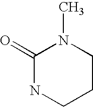 | |
| A9.018 | CF₃ | CH₂ | 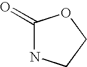 | |
| A9.019 | CF₃ | CH₂ | 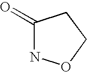 | |
| A9.020 | CF₃ | CH₂ | 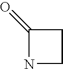 | |
| A9.021 | CF₃ | CH₂ | 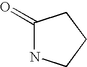 | |
| A9.022 | CF₃ | CH₂ | 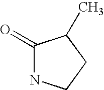 | |
| A9.023 | CF₃ | CH₂ | 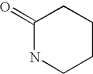 | |

TABLE A10
Compounds of formula IAa₁₀:

| | | | | (IAa₉) |
|---|---|---|---|---|
| 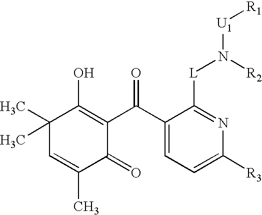 | | | | |
| Comp. No. | R₃ | L | 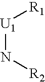 | Phys. data |
| A10.001 | CF₃ | CH₂ | 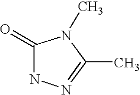 | |
| A10.002 | CF₂H | CH₂ | 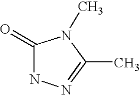 | |
| A10.003 | CF₃ | CH₂ | 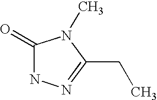 | |
| A10.004 | CF₃ | CH₂ | 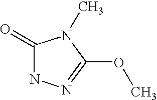 | |
| A10.005 | CF₃ | CH₂ | 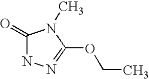 | |
| A10.006 | CF₃ | CH₂ | 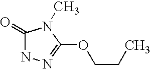 | |
| A10.007 | CF₃ | CH₂ | 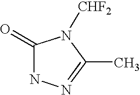 | |
| A10.008 | CF₃ | CH₂ | 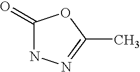 | |
| A10.009 | CF₃ | CH₂ | 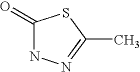 | |
| A10.010 | CF₃ | CH₂ | 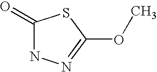 | |
| A10.011 | CF₃ | CH₂ | 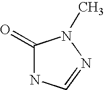 | |
| A10.012 | CF₃ | CH₂ | 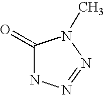 | |
| A10.013 | CF₃ | CH₂ | 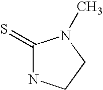 | |
| A10.014 | CF₃ | CH₂ | 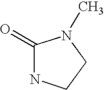 | |
| A10.015 | CF₃ | CH₂ | 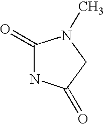 | |
| A10.016 | CF₃ | CH₂ | 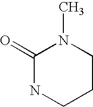 | |
| A10.017 | CF₃ | CH₂ | 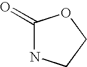 | |
| A10.018 | CF₃ | CH₂ | 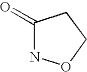 | |
| A10.019 | CF₃ | CH₂ | 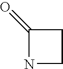 | |
| A10.020 | CF₃ | CH₂ | 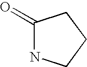 | |
| A10.021 | CF₃ | CH₂ | 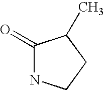 | |
| A10.022 | CF₃ | CH₂ | 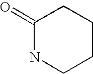 | |

TABLE B1
Compounds of formula IAb₁:

| | |
|---|---|
| | (IAb₁) |
| 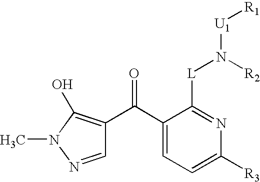 | |

| Comp. No. | R₃ | L | 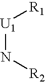 | Phys. data |
|---|---|---|---|---|
| B1.001 | CF₃ | CH₂ | 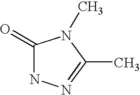 | solid |
| B1.002 | CF₂H | CH₂ | 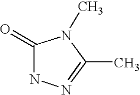 | |
| B1.003 | CF₃ | CH₂ | 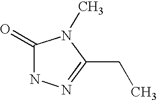 | |
| B1.004 | CF₃ | CH₂ | 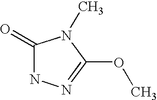 | solid |
| B1.005 | CF₃ | CH₂ | 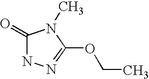 | solid |
| B1.006 | CF₃ | CH₂ | 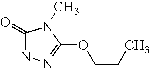 | |
| B1.007 | CF₃ | CH₂ | 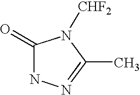 | |
| B1.008 | CF₃ | CH₂ | 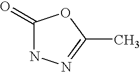 | m.p.: 173°C. |
| B1.009 | CF₃ | CH₂ | 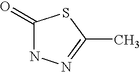 | |
| B1.010 | CF₃ | CH₂ | 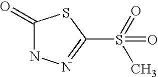 | |
| B1.011 | CF₃ | CH₂OCH₂CH₂ | 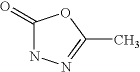 | |
| B1.012 | CF₃ | CH₂OCH₂CH₂ | 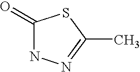 | |
| B1.013 | CF₃ | CH₂ | 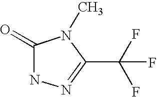 | |
| B1.014 | CF₂Cl | CH₂ | 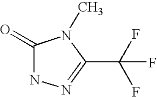 | |
| B1.015 | CF₂H | CH₂ | 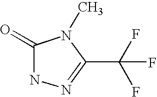 | |
| B1.016 | CF₃ | CH₂OCH₂CH₂ | 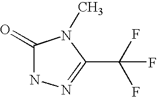 | |
| B1.017 | CF₂Cl | CH₂OCH₂CH₂ | 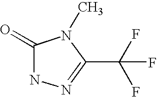 | |
| B1.018 | CHF₂ | CH₂OCH₂CH₂ | 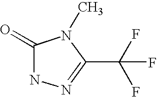 | |
| B1.019 | CF₂Cl | CH₂ | 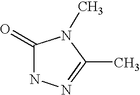 | |
| B1.020 | CF₃ | CH₂OCH₂CH₂ | 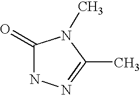 | |
| B1.021 | CF₂Cl | CH₂OCH₂CH₂ | 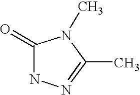 | |
| B1.022 | CHF₂ | CH₂OCH₂CH₂ | 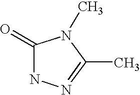 | |
| B1.023 | CF₂Cl | CH₂ | 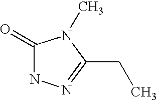 | |
| B1.024 | CHF₂ | CH₂ | 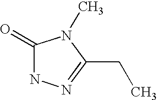 | |
| B1.025 | CF₃ | CH₂OCH₂CH₂ | 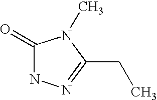 | |
| B1.026 | CF₂Cl | CH₂OCH₂CH₂ | 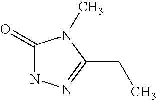 | |
| B1.027 | CHF₂ | CH₂OCH₂CH₂ | 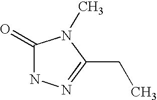 | |
| B1.028 | CF₂Cl | CH₂ | 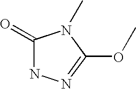 | |
| B1.029 | CHF₂ | CH₂ | 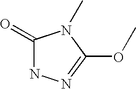 | |
| B1.030 | CF₃ | CH₂OCH₂CH₂ | 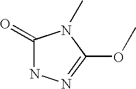 | |
| B1.031 | CF₂Cl | CH₂OCH₂CH₂ | 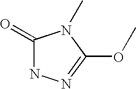 | |
| B1.032 | CHF₂ | CH₂OCH₂CH₂ | 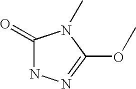 | |
| B1.033 | CF₃ | CH₂ | 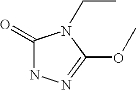 | solid |
| B1.034 | CF₂Cl | CH₂ | 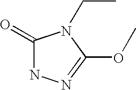 | |
| B1.035 | CHF₂ | CH₂ | 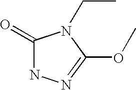 | |
| B1.036 | CF₃ | CH₂OCH₂CH₂ | 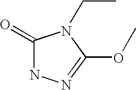 | |
| B1.037 | CF₂Cl | CH₂OCH₂CH₂ | 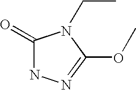 | |
| B1.038 | CHF₂ | CH₂OCH₂CH₂ | 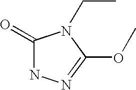 | |
| B1.039 | CF₃ | CH₂ | 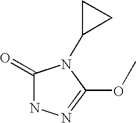 | solid |
| B1.040 | CF₂Cl | CH₂ | 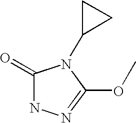 | |
| B1.041 | CHF₂ | CH₂ | 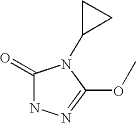 | |
| B1.042 | CF₃ | CH₂OCH₂CH₂ | 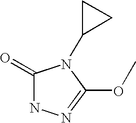 | |
| B1.043 | CF₂Cl | CH₂OCH₂CH₂ | 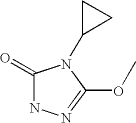 | |
| B1.044 | CHF₂ | CH₂OCH₂CH₂ | 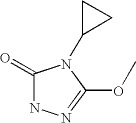 | |
| B1.050 | CF₃ | CH₂ | 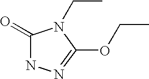 | solid |
| B1.051 | CF₂Cl | CH₂ | 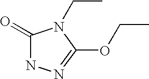 | |
| B1.052 | CHF₂ | CH₂ | 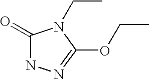 | |
| B1.053 | CF₃ | CH₂OCH₂CH₂ | 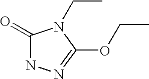 | |
| B1.054 | CF₂Cl | CH₂OCH₂CH₂ | 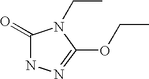 | |
| B1.055 | CHF₂ | CH₂OCH₂CH₂ | 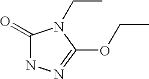 | |
| B1.056 | CF₃ | CH₂ | 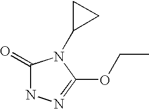 | solid |
| B1.057 | CF₂Cl | CH₂ | 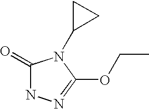 | |
| B1.058 | CHF₂ | CH₂ | 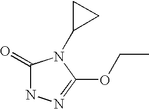 | |
| B1.059 | CF₃ | CH₂OCH₂CH₂ | 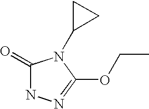 | |
| B1.060 | CF₂Cl | CH₂OCH₂CH₂ | 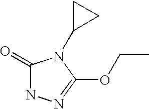 | |
| B1.061 | CHF₂ | CH₂OCH₂CH₂ | 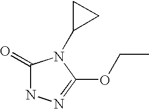 | |
| B1.062 | CF₃ | CH₂ | 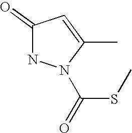 | m.p.: 173°C. |
| B1.063 | CF₂Cl | CH₂ | 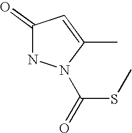 | |
| B1.064 | CHF₂ | CH₂ | 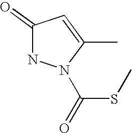 | |
| B1.065 | CF₃ | CH₂OCH₂CH₂ | 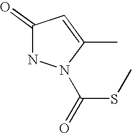 | |
| B1.066 | CF₂Cl | CH₂OCH₂CH₂ | 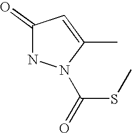 | |
| B1.067 | CHF₂ | CH₂OCH₂CH₂ | 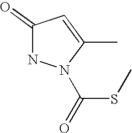 | |
| B1.068 | CF₃ | CH₂ | 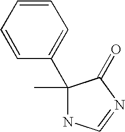 | |
| B1.069 | CF₂Cl | CH₂ | 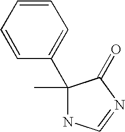 | |
| B1.070 | CHF₂ | CH₂ | 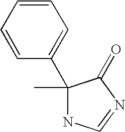 | |
| B1.071 | CF₃ | CH₂OCH₂CH₂ | 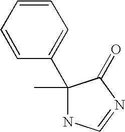 | |
| B1.072 | CF₂Cl | CH₂OCH₂CH₂ | 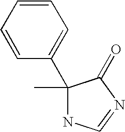 | |
| B1.073 | CHF₂ | CH₂OCH₂CH₂ | 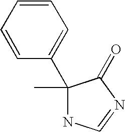 | |
| B1.074 | CF₃ | CH₂ | 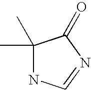 | |
| B1.075 | CF₂Cl | CH₂ | 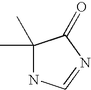 | |
| B1.076 | CHF₂ | CH₂ | 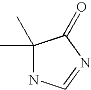 | |
| B1.077 | CF₃ | CH₂OCH₂CH₂ | 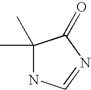 | |
| B1.078 | CF₂Cl | CH₂OCH₂CH₂ | 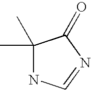 | |
| B1.079 | CHF₂ | CH₂OCH₂CH₂ | 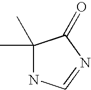 | |
| B1.080 | CF₃ | CH₂ | 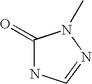 | solid |
| B1.081 | CF₂Cl | CH₂ | 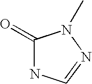 | |
| B1.082 | CHF₂ | CH₂ | 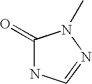 | |
| B1.083 | CF₃ | CH₂OCH₂CH₂ | 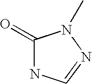 | |
| B1.084 | CF₂Cl | CH₂OCH₂CH₂ | 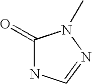 | |
| B1.085 | CHF₂ | CH₂OCH₂CH₂ | 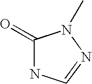 | |
| B1.086 | CF₂Cl | CH₂ | 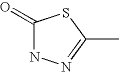 | |
| B1.087 | CHF₂ | CH₂ | 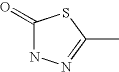 | |
| B1.088 | CF₃ | CH₂OCH₂CH₂ | 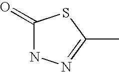 | |
| B1.089 | CF₂Cl | CH₂OCH₂CH₂ | 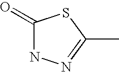 | |
| B1.090 | CHF₂ | CH₂OCH₂CH₂ | 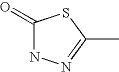 | |
| B1.091 | CF₂Cl | CH₂ | 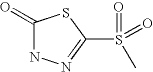 | |
| B1.092 | CHF₂ | CH₂ | 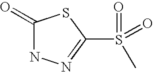 | |
| B1.093 | CF₃ | CH₂OCH₂CH₂ | 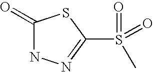 | |
| B1.094 | CF₂Cl | CH₂OCH₂CH₂ | 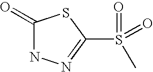 | |
| B1.095 | CHF₂ | CH₂OCH₂CH₂ | 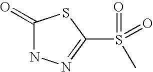 | |
| B1.096 | CF₃ | CH₂ | 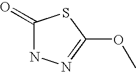 | solid |
| B1.097 | CF₂Cl | CH₂ | 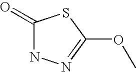 | |
| B1.098 | CHF₂ | CH₂ | 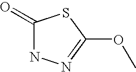 | |
| B1.099 | CF₃ | CH₂OCH₂CH₂ | 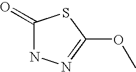 | |
| B1.100 | CF₂Cl | CH₂OCH₂CH₂ | 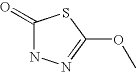 | |
| B1.101 | CHF₂ | CH₂OCH₂CH₂ | 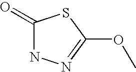 | |
| B1.102 | CF₂Cl | CH₂ | 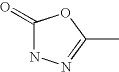 | |
| B1.103 | CHF₂ | CH₂ | 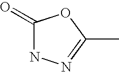 | |
| B1.104 | CF₂ | CH₂OCH₂CH₂ | 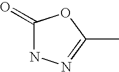 | |
| B1.105 | CF₂Cl | CH₂OCH₂CH₂ | 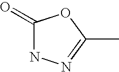 | |
| B1.106 | CHF₂ | CH₂OCH₂CH₂ | 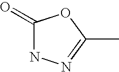 | |
| B1.107 | CF₃ | CH₂ | 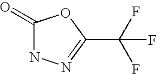 | |
| B1.108 | CF₂Cl | CH₂ | 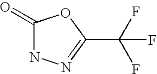 | |
| B1.109 | CHF₂ | CH₂ | 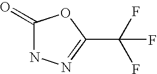 | |
| B1.110 | CF₃ | CH₂OCH₂CH₂ | 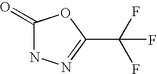 | |
| B1.111 | CF₂Cl | CH₂OCH₂CH₂ | 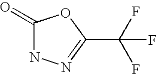 | |
| B1.112 | CHF₂ | CH₂OCH₂CH₂ | 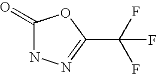 | |
| B1.113 | CF₃ | CH₂ | 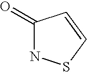 | |
| B1.114 | CF₂Cl | CH₂ | 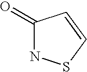 | |
| B1.115 | CHF₂ | CH₂ | 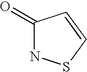 | |
| B1.116 | CF₃ | CH₂OCH₂CH₂ | 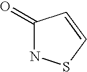 | |
| B1.117 | CF₂Cl | CH₂OCH₂CH₂ | 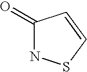 | |
| B1.118 | CHF₂ | CH₂OCH₂CH₂ | 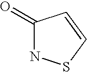 | |
| B1.119 | CF₃ | CH₂ | 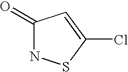 | |
| B1.120 | CF₂Cl | CH₂ | 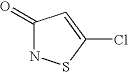 | |
| B1.121 | CHF₂ | CH₂ | 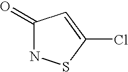 | |
| B1.122 | CF₃ | CH₂OCH₂CH₂ | 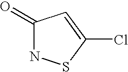 | |
| B1.123 | CF₂Cl | CH₂OCH₂CH₂ | 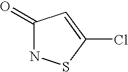 | |
| B1.124 | CHF₂ | CH₂OCH₂CH₂ | 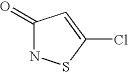 | |
| B1.125 | CF₃ | CH₂ | 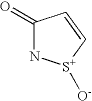 | |
| B1.126 | CF₂Cl | CH₂ | 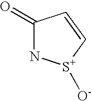 | |
| B1.127 | CHF₂ | CH₂ | 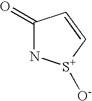 | |
| B1.128 | CF₃ | CH₂OCH₂CH₂ | 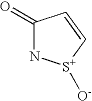 | |
| B1.129 | CF₂Cl | CH₂OCH₂CH₂ | 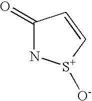 | |
| B1.130 | CHF₂ | CH₂OCH₂CH₂ | 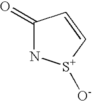 | |
| B1.131 | CF₃ | CH₂ | 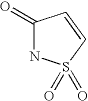 | |
| B1.132 | CF₂Cl | CH₂ | 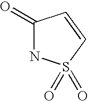 | |
| B1.133 | CHF₂ | CH₂ | 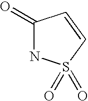 | |
| B1.134 | CF₃ | CH₂OCH₂CH₂ | 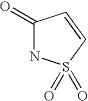 | |
| B1.135 | CF₂Cl | CH₂OCH₂CH₂ | 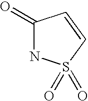 | |
| B1.136 | CHF₂ | CH₂OCH₂CH₂ | 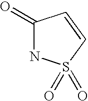 | |
| B1.137 | CF₃ | CH₂ | 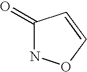 | |
| B1.138 | CF₂Cl | CH₂ | 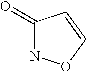 | |
| B1.139 | CHF₂ | CH₂ | 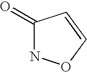 | |
| B1.140 | CF₃ | CH₂OCH₂CH₂ | 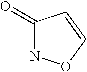 | |
| B1.141 | CF₂Cl | CH₂OCH₂CH₂ | 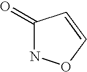 | |
| B1.142 | CHF₂ | CH₂OCH₂CH₂ | 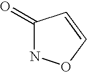 | |
| B1.143 | CF₂Cl | CH₂ | 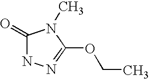 | |
| B1.144 | CHF₂ | CH₂ | 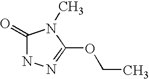 | |
| B1.145 | CF₃ | CH₂OCH₂CH₂ | 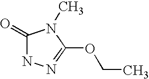 | |
| B1.146 | CF₂Cl | CH₂OCH₂CH₂ | 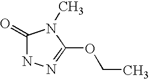 | |
| B1.147 | CHF₂ | CH₂OCH₂CH₂ | 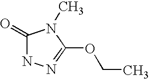 | |
| B1.148 | CF₂Cl | CH₂ | 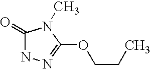 | |
| B1.149 | CHF₂ | CH₂ | 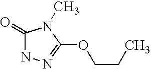 | |
| B1.150 | CF₃ | CH₂OCH₂CH₂ | 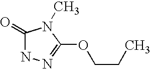 | |
| B1.151 | CF₂Cl | CH₂OCH₂CH₂ | 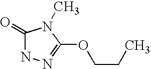 | |
| B1.152 | CHF₂ | CH₂OCH₂CH₂ | 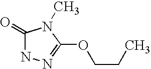 | |
| B1.153 | CF₂Cl | CH₂ | 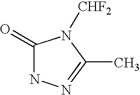 | |
| B1.154 | CHF₂ | CH₂ | 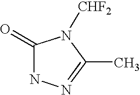 | |
| B1.155 | CF₃ | CH₂OCH₂CH₂ | 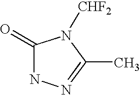 | |
| B1.156 | CF₂Cl | CH₂OCH₂CH₂ | 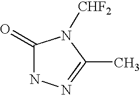 | |
| B1.157 | CHF₂ | CH₂OCH₂CH₂ | 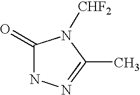 | |
| B1.158 | CF₃ | CH₂ | 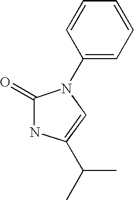 | resin |
| B1.159 | CF₂Cl | CH₂ | 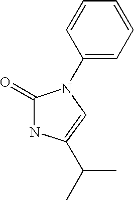 | |
| B1.160 | CHF₂ | CH₂ | 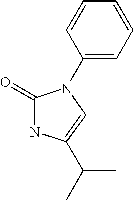 | |
| B1.161 | CF₃ | CH₂OCH₂CH₂ | 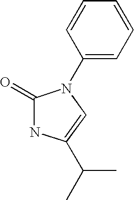 | |
| B1.162 | CF₂Cl | CH₂OCH₂CH₂ | 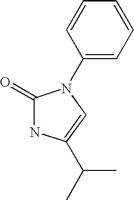 | |
| B1.163 | CHF₂ | CH₂OCH₂CH₂ | 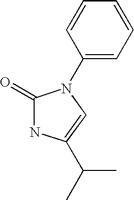 | |
| B1.164 | CF₃ | CH₂ | 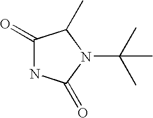 | |
| B1.165 | CF₂Cl | CH₂ | 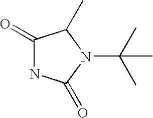 | |
| B1.166 | CHF₂ | CH₂ | 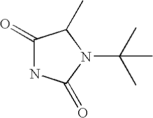 | |
| B1.167 | CF₃ | CH₂OCH₂CH₂ | 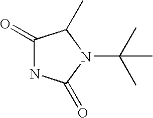 | |
| B1.168 | CF₂Cl | CH₂OCH₂CH₂ | 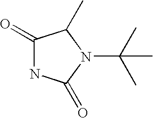 | |
| B1.169 | CHF₂ | CH₂OCH₂CH₂ | 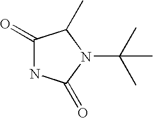 | |
| B1.170 | CF₃ | CH₂ | 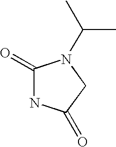 | m.p.: 171°C. |
| B1.171 | CF₂Cl | CH₂ | 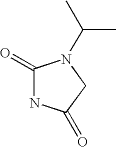 | |
| B1.172 | CHF₂ | CH₂ | 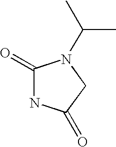 | |
| B1.173 | CF₃ | CH₂OCH₂CH₂ | 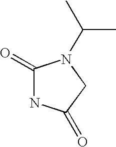 | |
| B1.174 | CF₂Cl | CH₂OCH₂CH₂ | 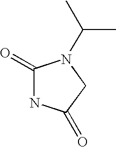 | |
| B1.175 | CHF₂ | CH₂OCH₂CH₂ | 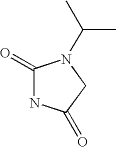 | |
| B1.176 | CF₃ | CH₂ | 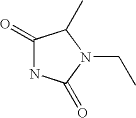 | solid |
| B1.177 | CF₂Cl | CH₂ | 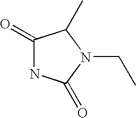 | |
| B1.178 | CHF₂ | CH₂ | 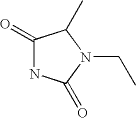 | |
| B1.179 | CF₃ | CH₂OCH₂CH₂ | 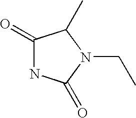 | |
| B1.180 | CF₂Cl | CH₂OCH₂CH₂ | 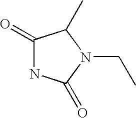 | |
| B1.181 | CHF₂ | CH₂OCH₂CH₂ | 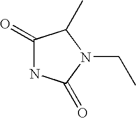 | |
| B1.182 | CF₃ | CH₂ | 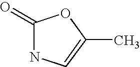 | |
| B1.183 | CF₂Cl | CH₂ | 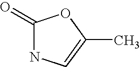 | |
| B1.184 | CHF₂ | CH₂ | 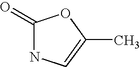 | |
| B1.185 | CF₃ | CH₂OCH₂CH₂ | 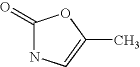 | |
| B1.186 | CF₂Cl | CH₂OCH₂CH₂ | 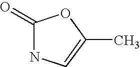 | |
| B1.187 | CHF₂ | CH₂OCH₂CH₂ | 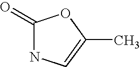 | |
| B1.188 | CF₃ | CH₂ | 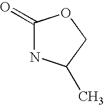 | solid |
| B1.189 | CF₂Cl | CH₂ | 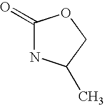 | |
| B1.190 | CHF₂ | CH₂ | 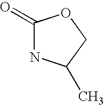 | |
| B1.191 | CF₃ | CH₂OCH₂CH₂ | 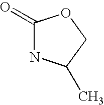 | |
| B1.192 | CF₂Cl | CH₂OCH₂CH₂ | 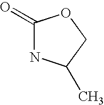 | |
| B1.193 | CHF₂ | CH₂OCH₂CH₂ | 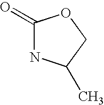 | |
| B1.194 | CF₃ | CH₂ | 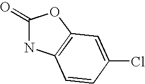 | solid |
| B1.195 | CF₂Cl | CH₂ | 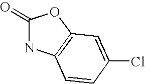 | |
| B1.196 | CHF₂ | CH₂ | 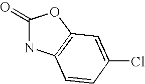 | |
| B1.197 | CF₃ | CH₂ | 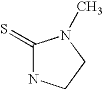 | |
| B1.198 | CF₂Cl | CH₂ | 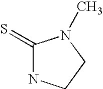 | |
| B1.199 | CHF₂ | CH₂ | 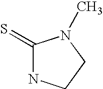 | |
| B1.200 | CF₃ | CH₂OCH₂CH₂ | 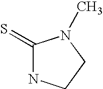 | |
| B1.201 | CF₂Cl | CH₂OCH₂CH₂ | 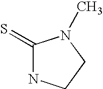 | |
| B1.202 | CHF₂ | CH₂OCH₂CH₂ | 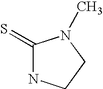 | |
| B1.203 | CF₃ | CH₂ | 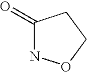 | |
| B1.204 | CF₂Cl | CH₂ | 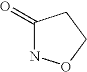 | |
| B1.205 | CHF₂ | CH₂ | 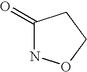 | |
| B1.206 | CF₃ | CH₂ | 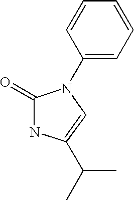 | |
| B1.207 | CF₃ | CH₂ | 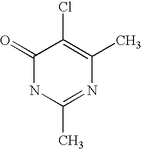 | |
| B1.208 | CF₂Cl | CH₂ | 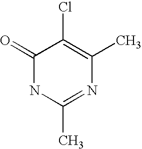 | |
| B1.209 | CF₃ | CH₂ | 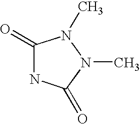 | |
| B1.210 | CF₂Cl | CH₂ | 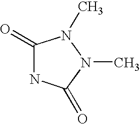 | |
| B1.211 | CF₃ | CH₂ | 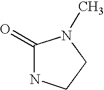 | solid |
| B1.212 | CF₂Cl | CH₂ | 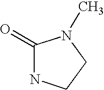 | |
| B1.213 | CHF₂ | CH₂ | 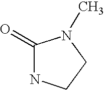 | |
| B1.214 | CF₃ | CH₂OCH₂CH₂ | 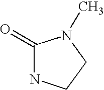 | |
| B1.215 | CF₂Cl | CH₂OCH₂CH₂ | 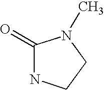 | |
| B1.216 | CHF₂ | CH₂OCH₂CH₂ | 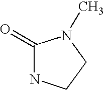 | |
| B1.217 | CH₂ | CF₃ | 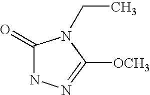 | |
| B1.218 | CH₂ | CF₂Cl | 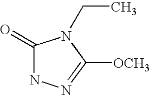 | |
| B1.219 | CH₂ | CHF₂ | 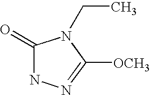 | |
| B1.220 | CH₂OCH_{2 CH}₂ | CF₃ | 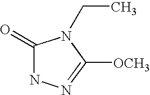 | |
| B1.221 | CH₂OCH_{2 CH}₂ | CF₂Cl | 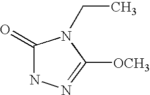 | |
| B1.222 | CH₂OCH_{2 CH}₂ | CHF₂ | 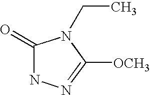 | |
| B1.223 | CF₃ | CH₂ | 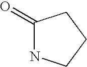 | solid |
| B1.224 | CF₃ | CH₂OCH₂CH₂ | 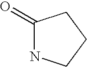 | resin |
| B1.225 | CF₃ | CH₂ | 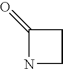 | solid |
| B1.226 | CF₃ | CH₂ | 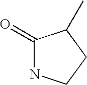 | solid |
| B1.227 | CF₃ | CH₂ | 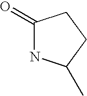 | solid |
| B1.228 | CClF₂ | CH₂ | 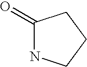 | |
| B1.229 | CClF₂ | CH₂ | 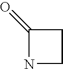 | |
| B1.230 | CClF₂ | CH₂ | 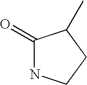 | |
| B1.231 | CClF₂ | CH₂ | 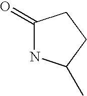 | |
| B1.232 | CHF₂ | CH₂ | 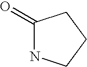 | |
| B1.233 | CHF₂ | CH₂ | 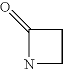 | |
| B1.234 | CHF₂ | CH₂ | 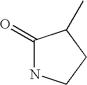 | |
| B1.235 | CHF₂ | CH₂ | 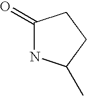 | |
| B1.236 | CF₃ | CH₂ | 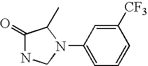 | resin |
| B1.237 | CHF₂ | CH₂ | 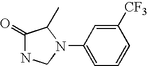 | |
| B1.238 | CF₃ | CH₂ | 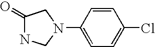 | solid |
| B1.239 | CHF₂ | CH₂ | 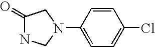 | |
| B1.240 | CF₃ | CH₂ | 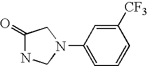 | m.p.: 192°C. |
| B1.241 | CHF₂ | CH₂ | 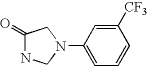 | |
| B1.242 | CF₃ | CH₂ | 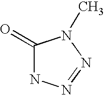 | |
| B1.243 | CF₃ | CH₂ | 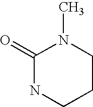 | |
| B1.244 | CF₃ | CH₂ | 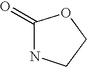 | |

TABLE B2
Compounds of formula IAb₂:

| | |
|---|---|
| | (IAb₂) |
| 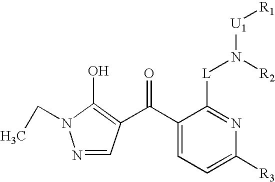 | |

| | | | | Phys. |
|---|---|---|---|---|
| Comp. No. | R₃ | L | 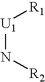 | data |
| B2.001 | CF₃ | CH₂ | 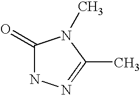 | |
| B2.002 | CF₂H | CH₂ | 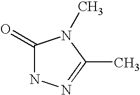 | |
| B2.003 | CF₃ | CH₂ | 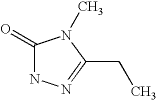 | |
| B2.004 | CF₃ | CH₂ | 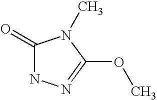 | |
| B2.005 | CF₃ | CH₂ | 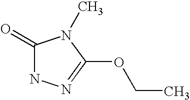 | |
| B2.006 | CF₃ | CH₂ | 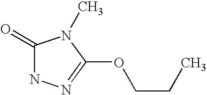 | |
| B2.007 | CF₃ | CH₂ | 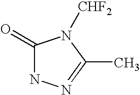 | |
| B2.008 | CF₃ | CH₂ | 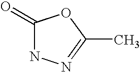 | |
| B2.009 | CF₃ | CH₂ | 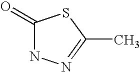 | |
| B2.010 | CF₃ | CH₂ | 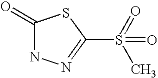 | |
| B2.011 | CF₃ | CH₂ | 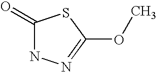 | |
| B2.012 | CF₃ | CH₂ | 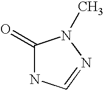 | |
| B2.013 | CF₃ | CH₂ | 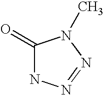 | |
| B2.014 | CF₃ | CH₂ | 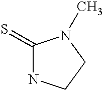 | |
| B2.015 | CF₃ | CH₂ | 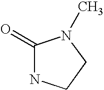 | |
| B2.016 | CF₃ | CH₂ | 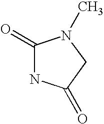 | |
| B2.017 | CF₃ | CH₂ | 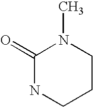 | |
| B2.018 | CF₃ | CH₂ | 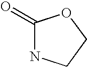 | |
| B2.019 | CF₃ | CH₂ | 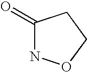 | |
| B2.020 | CF₃ | CH₂ | 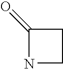 | |
| B2.021 | CF₃ | CH₂ | 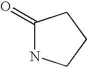 | |
| B2.022 | CF₃ | CH₂ | 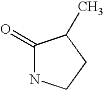 | |
| B2.023 | CF₃ | CH₂ | 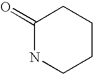 | |

TABLE B3
Compounds of formula IAb₃:

| | |
|---|---|
| | (IAb₃) |
| 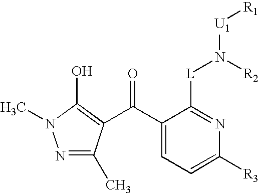 | |

| | | | | Phys. |
|---|---|---|---|---|
| Comp. No. | R₃ | L | 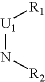 | data |
| B3.001 | CF₃ | CH₂ | 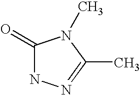 | |
| B3.002 | CF₂H | CH₂ | 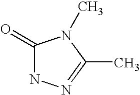 | |
| B3.003 | CF₃ | CH₂ | 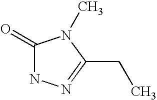 | |
| B3.004 | CF₃ | CH₂ | 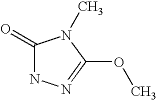 | |
| B3.005 | CF₃ | CH₂ | 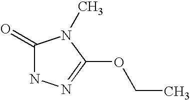 | |
| B3.006 | CF₃ | CH₂ | 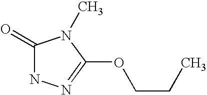 | |
| B3.007 | CF₃ | CH₂ | 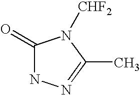 | |
| B3.008 | CF₃ | CH₂ | 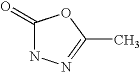 | |
| B3.009 | CF₃ | CH₂ | 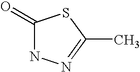 | |
| B3.010 | CF₃ | CH₂ | 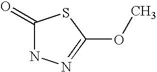 | |
| B3.011 | CF₃ | CH₂ | 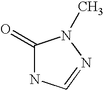 | |
| B3.012 | CF₃ | CH₂ | 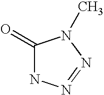 | |
| B3.013 | CF₃ | CH₂ | 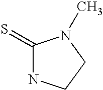 | |
| B3.014 | CF₃ | CH₂ | 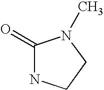 | |
| B3.015 | CF₃ | CH₂ | 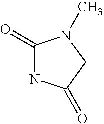 | |
| B3.016 | CF₃ | CH₂ | 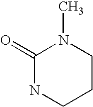 | |
| B3.017 | CF₃ | CH₂ | 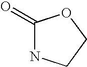 | |
| B3.018 | CF₃ | CH₂ | 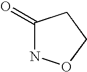 | |
| B3.019 | CF₃ | CH₂ | 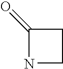 | |
| B3.020 | CF₃ | CH₂ | 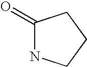 | |
| B3.021 | CF₃ | CH₂ | 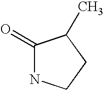 | |
| B3.022 | CF₃ | CH₂ | 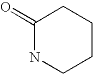 | |

TABLE C1
Compounds of formula IAc₁:

| | |
|---|---|
| | (IAc₁) |
| 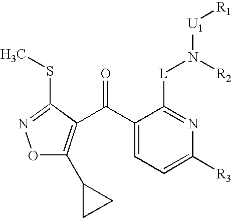 | |

| | | | | Phys. |
|---|---|---|---|---|
| Comp. No. | R₃ | L | 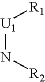 | data |
| C1.001 | CF₃ | CH₂ | 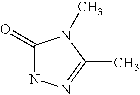 | |
| C1.002 | CF₂H | CH₂ | 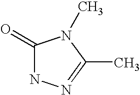 | |
| C1.003 | CF₃ | CH₂ | 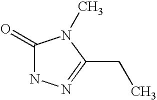 | |
| C1.004 | CF₃ | CH₂ | 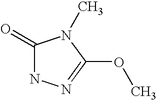 | |
| C1.005 | CF₃ | CH₂ | 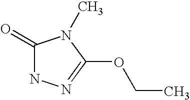 | |
| C1.006 | CF₃ | CH₂ | 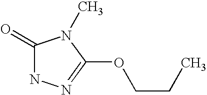 | |
| C1.007 | CF₃ | CH₂ | 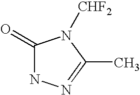 | |
| C1.008 | CF₃ | CH₂ | 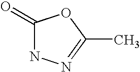 | |
| C1.009 | CF₃ | CH₂ | 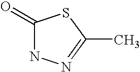 | |
| C1.010 | CF₃ | CH₂ | 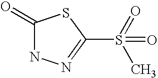 | |
| C1.011 | CF₃ | CH₂ | 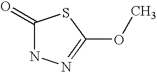 | |
| C1.012 | CF₃ | CH₂ | 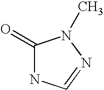 | |
| C1.013 | CF₃ | CH₂ | 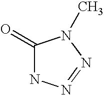 | |
| C1.014 | CF₃ | CH₂ | 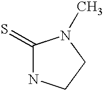 | |
| C1.015 | CF₃ | CH₂ | 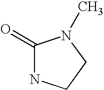 | |
| C1.016 | CF₃ | CH₂ | 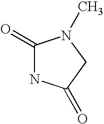 | |
| C1.017 | CF₃ | CH₂ | 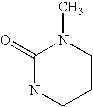 | |
| C1.018 | CF₃ | CH₂ | 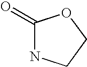 | |
| C1.019 | CF₃ | CH₂ | 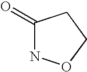 | |
| C1.020 | CF₃ | CH₂ | 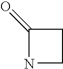 | |
| C1.021 | CF₃ | CH₂ | 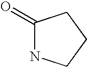 | |
| C1.022 | CF₃ | CH₂ | 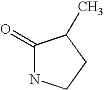 | |
| C1.023 | CF₃ | CH₂ | 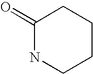 | |

TABLE C2
Compounds of formula IAc₂:

| | |
|---|---|
| | (IAc₂) |
| 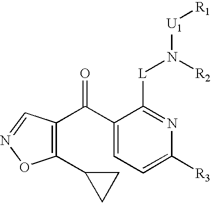 | |

| | | | | Phys. |
|---|---|---|---|---|
| Comp. No. | R₃ | L | 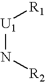 | data |
| C2.001 | CF₃ | CH₂ | 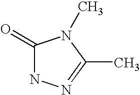 | |
| C2.002 | CF₂H | CH₂ | 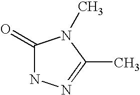 | |
| C2.003 | CF₃ | CH₂ | 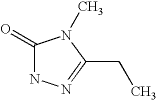 | |
| C2.004 | CF₃ | CH₂ | 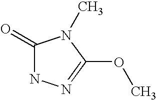 | |
| C2.005 | CF₃ | CH₂ | 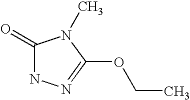 | |
| C2.006 | CF₃ | CH₂ | 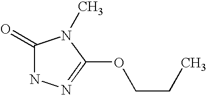 | |
| C2.007 | CF₃ | CH₂ | 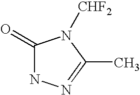 | |
| C2.008 | CF₃ | CH₂ | 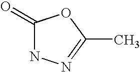 | |
| C2.009 | CF₃ | CH₂ | 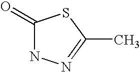 | |
| C2.010 | CF₃ | CH₂ | 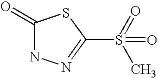 | |
| C2.011 | CF₃ | CH₂ | 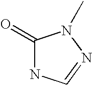 | |
| C2.011 | CF₃ | CH₂ | 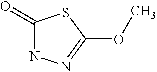 | |
| C2.012 | CF₃ | CH₂ | 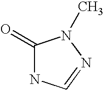 | |
| C2.013 | CF₃ | CH₂ | 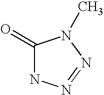 | |
| C2.014 | CF₃ | CH₂ | 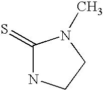 | |
| C2.015 | CF₃ | CH₂ | 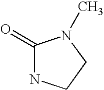 | |
| C2.016 | CF₃ | CH₂ | 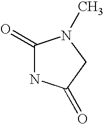 | |
| C2.017 | CF₃ | CH₂ | 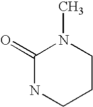 | |
| C2.018 | CF₃ | CH₂ | 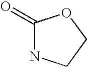 | |
| C2.019 | CF₃ | CH₂ | 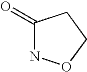 | |
| C2.020 | CF₃ | CH₂ | 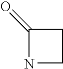 | |
| C2.021 | CF₃ | CH₂ | 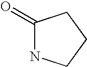 | |
| C2.022 | CF₃ | CH₂ | 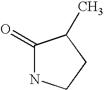 | |
| C2.023 | CF₃ | CH₂ | 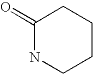 | |

TABLE D1
Compounds of formula IAd:

| | |
|---|---|
| | (IAd₁) |
| 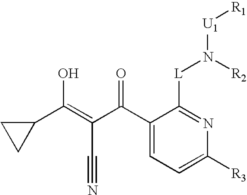 | |

| | | | | Phys. |
|---|---|---|---|---|
| Comp. No. | R₃ | L | 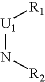 | data |
| D1.001 | CF₃ | CH₂ | 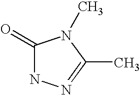 | |
| D1.002 | CF₂H | CH₂ | 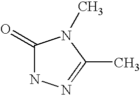 | |
| D1.003 | CF₃ | CH₂ | 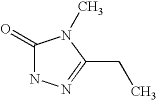 | |
| D1.004 | CF₃ | CH₂ | 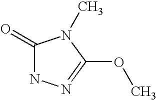 | |
| D1.005 | CF₃ | CH₂ | 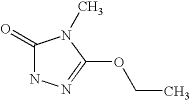 | |
| D1.006 | CF₃ | CH₂ | 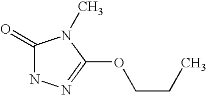 | |
| D1.007 | CF₃ | CH₂ | 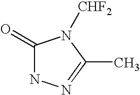 | |
| D1.008 | CF₃ | CH₂ | 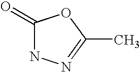 | |
| D1.009 | CF₃ | CH₂ | 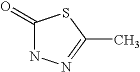 | |
| D1.010 | CF₃ | CH₂ | 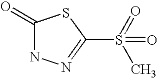 | |
| D1.011 | CF₃ | CH₂ | 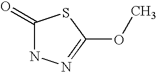 | |
| D1.012 | CF₃ | CH₂ | 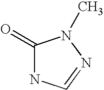 | |
| D1.013 | CF₃ | CH₂ | 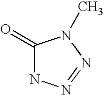 | |
| D1.014 | CF₃ | CH₂ | 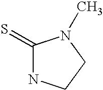 | |
| D1.015 | CF₃ | CH₂ | 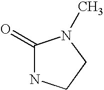 | |
| D1.016 | CF₃ | CH₂ | 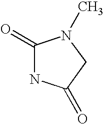 | |
| D1.017 | CF₃ | CH₂ | 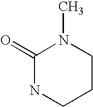 | |
| D1.018 | CF₃ | CH₂ | 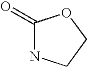 | |
| D1.019 | CF₃ | CH₂ | 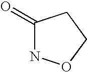 | |
| D1.020 | CF₃ | CH₂ | 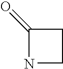 | |
| D1.021 | CF₃ | CH₂ | 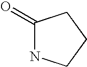 | |
| D1.022 | CF₃ | CH₂ | 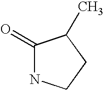 | |
| D1.023 | CF₃ | CH₂ | 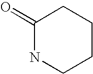 | |

TABLE S1
Compounds of formula II:

| | |
|---|---|
| | (IIa) |
| 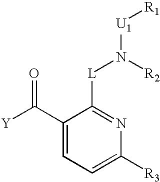 | |

| Comp. No. | Y | R₃ | L | 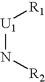 | Phys. data |
|---|---|---|---|---|---|
| S1.001 (P7) | OH | CF₂Cl | CH₂ | 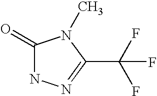 | amorphous crystals |
| S1.002 | OC₂H₅ | CF₃ | CH₂ | 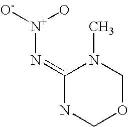 | 132-133° C. |
| S1.003 | OH | CF₃ | CH₂ | 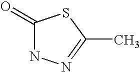 | amorphous crystals |
| S1.004 (P4) | OH | CF₃ | CH₂ | 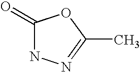 | amorphous crystals |
| S1.005 | OC₂H₅ | CF₃ | CH₂ | 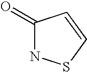 | solid |
| S1.006 | OH | CF₃ | CH₂ | 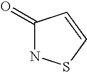 | solid |
| S1.007 | OC₂H₅ | CF₃ | CH₂ | 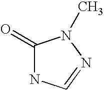 | solid |
| S1.008 | OH | CF₃ | CH₂ | 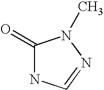 | m.p.: 210° C. |
| S1.009 | OC₂H₅ | CF₃ | CH₂ | 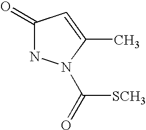 | solid |
| S1.010 | OH | CF₃ | CH₂ | 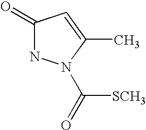 | m:p.: 145° C. |
| S1.011 | OC₂H₅ | CF₃ | CH₂ | 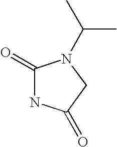 | solid |
| S1.012 | OH | CF₃ | CH₂ | 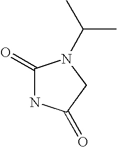 | m.p.: 189° C. |
| S1.013 | OC₂H₅ | CF₃ | CH₂ | 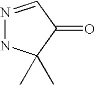 | m.p.: 91° C. |
| S1.014 | OH | CF₃ | CH₂ | 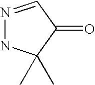 | solid |
| S1.015 | OC₂H₅ | CF₃ | CH₂ | 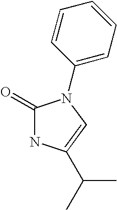 | m.p.: 109° C. |
| S1.016 | OH | CF₃ | CH₂ | 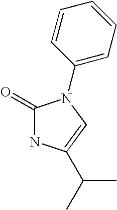 | m.p.: 191° C. |
| S1.017 | OC₂H₅ | CF₃ | CH₂ | 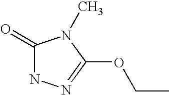 | waxy |
| S1.018 | OH | CF₃ | CH₂ | 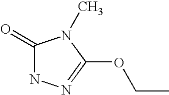 | solid |
| S1.019 | OC₂H₅ | CF₃ | CH₂ | 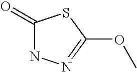 | m.p.: 82° C. |
| S1.020 | OH | CF₃ | CH₂ | 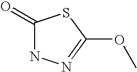 | m.p.: 142° C. |
| S1.021 | OC₂H₅ | CF₃ | CH₂ | 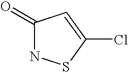 | resin |
| S1.022 | OH | CF₃ | CH₂ | 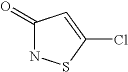 | solid |
| S1.023 | OC₂H₅ | CF₃ | CH₂ | 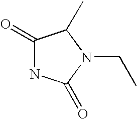 | m.p.: 114° C. |
| S1.024 | OH | CF₃ | CH₂ | 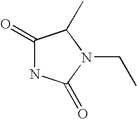 | m.p.: 165° C. |
| S1.025 | OC₂H₅ | CF₃ | CH₂ | 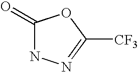 | |
| S1.026 | OH | CF₃ | CH₂ | 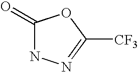 | m.p.: 128° C. |
| S1.027 | OC₂H₅ | CF₃ | CH₂ | 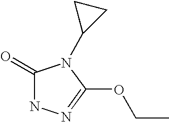 | m.p.: 123° C. |
| S1.028 | OH | CF₃ | CH₂ | 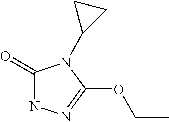 | m.p.: 166° C. |
| S1.029 | OC₂H₅ | CF₃ | CH₂ | 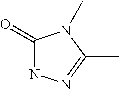 | m.p.: 116° C. |
| S1.030 | OH | CF₃ | CH₂ | 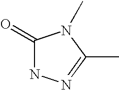 | m.p.: 174° C. |
| S1.031 | OC₂H₅ | CF₃ | CH₂ | 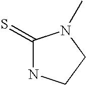 | solid |
| S1.032 | OH | CF₃ | CH₂ | 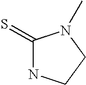 | m.p.: 184° C. |
| S1.033 | OC₂H₅ | CF₃ | CH₂ | 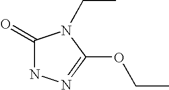 | solid |
| S1.034 | OH | CF₃ | CH₂ | 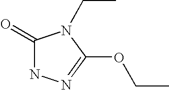 | solid |
| S1.035 | OC₂H₅ | CF₃ | CH₂ | 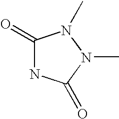 | solid |
| S1.036 | OH | CF₃ | CH₂ | 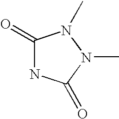 | solid |
| S1.037 | OC₂H₅ | CF₃ | CH₂ | 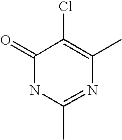 | solid |
| S1.038 | OH | CF₃ | CH₂ | 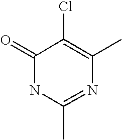 | solid |
| S1.039 | OC₂H₅ | CF₃ | CH₂ | 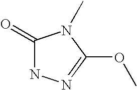 | solid |
| S1.040 | OH | CF₃ | CH₂ | 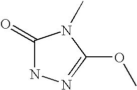 | solid |
| S1.041 | OC₂H₅ | CF₃ | CH₂ | 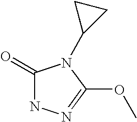 | solid |
| S1.042 | OH | CF₃ | CH₂ | 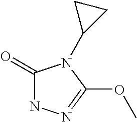 | solid |
| S1.043 | OC₂H₅ | CF₃ | CH₂ | 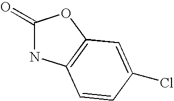 | solid |
| S1.044 | OH | CF₃ | CH₂ | 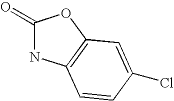 | solid |
| S1.045 | OC₂H₅ | CF₃ | CH₂ | 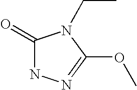 | solid |
| S1.046 | OH | CF₃ | CH₂ | 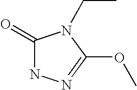 | solid |
| S1.047 (P6) | OH | CF₃ | CH₂ | 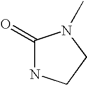 | solid |
| S1.048 | OH | CF₃ | CH₂ | 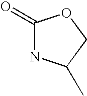 | solid |
| S1.049 | OH | CF₃ | CH₂ | 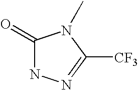 | crystalline |
| S1.050 | OC₂H₅ | CClF₂ | CH₂ | 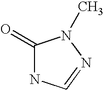 | m.p.: 87-88° C. |
| S1.051 | OH | CClF₂ | CH₂ | 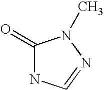 | m.p.: 180-182° C. |
| S1.052 | OC₂H₅ | CClF₂ | CH₂ | 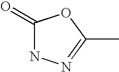 | |
| S1.053 | OH | CClF₂ | CH₂ | 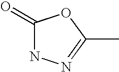 | m.p.: 173-174° C. |
| S1.054 | OC₂H₅ | CCHF₂ | CH₂ | 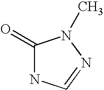 | |
| S1.055 | OH | CCHF₂ | CH₂ | 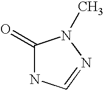 | |
| S1.056 | OC₂H₅ | CCHF₂ | CH₂ | 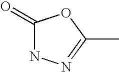 | resin |
| S1.057 | OH | CCHF₂ | CH₂ | 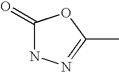 | |
| S1.058 | OC₂H₅ | CCHF₂ | CH₂ | 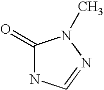 | |
| S1.059 | OH | CF₃ | CH₂ | 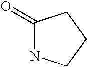 | solid |
| S1.060 | OH | CF₃ | CH₂OCH₂CH₂ | 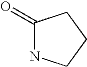 | solid |
| S1.061 | OH | CF₃ | CH₂ | 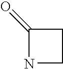 | solid |
| S1.062 | OH | CF₃ | CH₂ | 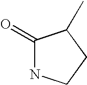 | solid |
| S1.063 | OH | CF₃ | CH₂ | 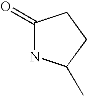 | solid |
| S1.064 | OH | CClF₂ | CH₂ | 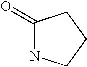 | |
| S1.065 | OH | CClF₂ | CH₂ | 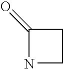 | |
| S1.066 | OH | CClF₂ | CH₂ | 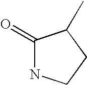 | |
| S1.067 | OH | CClF₂ | CH₂ | 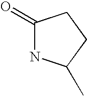 | |
| S1.068 | OH | CHF₂ | CH₂ | 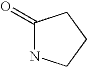 | |
| S1.069 | OH | CHF₂ | CH₂ | 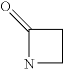 | |
| S1.070 | OH | CHF₂ | CH₂ | 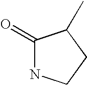 | |
| S1.071 | OH | CHF₂ | CH₂ | 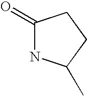 | |
| S1.072 | OC₂H₅ | CF₃ | CH₂ | 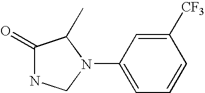 | m.p.: 122° C. |
| S1.073 | OH | CF₃ | CH₂ | 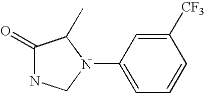 | m.p.: 182° C. |
| S1.074 | OC₂H₅ | CF₃ | CH₂ | 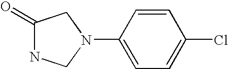 | m.p.: 132° C. |
| S1.075 | OH | CF₃ | CH₂ | 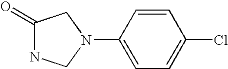 | m.p.: 255° C. |
| S1.076 | OC₂H₅ | CF₃ | CH₂ | 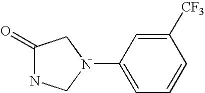 | m.p.: 113° C. |
| S1.077 | OH | CF₃ | CH₂ | 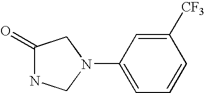 | m.p.: 228° C. |
| S1.078 (P5) | OC₂H₅ | CF₃ | CH₂ | 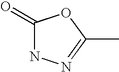 | amorphous crystals |
| S1.079 (P7) | OC₂H₅ | CF₂Cl | CH₂ | 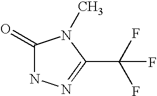 | resin |

### BIOLOGICAL EXAMPLES

### EXAMPLE B1

### Herbicidal Action Prior to Emergence of the Plants (Pre-Emergence Action)

Monocotyledonous and dicotyledonous test plants are sown in standard soil in plastic pots Immediately after sowing, the test compounds, in the form of an aqueous suspension (prepared from a 25% wettable powder (Example F3, b) according to WO 97/34485) or in the form of an emulsion (prepared from a 25% emulsifiable concentrate (Example F1, c)), are applied by spraying in a concentration corresponding to 125 g or 250 g of active ingredient/ha (500 litres of water/ha). The test plants are then grown in a greenhouse under optimum conditions. After a test duration of 3 weeks, the test is evaluated in accordance with a scale of nine ratings (10=total damage, 0=no action). Ratings of from 10 to 7 (especially from 10 to 8) indicate good to very good herbicidal action.

TABLE B1
Pre-emergence action of compounds of formula I:

| Ex. No. | gr. a.i./ha | Panicum | Echinochloa | Cyperus | Scirpus | Sida | Abutilon | Amaranthus | Chenopodium |
|---|---|---|---|---|---|---|---|---|---|
| A1.055 | 250 | 9 | 10 | 10 | 9 | 10 | 10 | 0 | 10 |
| A1.073 | 250 | 10 | 3 | 10 | 10 | 9 | 10 | 10 | 10 |
| A1.079 | 250 | 9 | 5 | 8 | 10 | 10 | 10 | 4 | 8 |
| A1.091 | 250 | 4 | 9 | 8 | 9 | 7 | 10 | 8 | 9 |
| A6.073 | 250 | 10 | 0 | 7 | 10 | 9 | 10 | 9 | 10 |
| A6.079 | 250 | 9 | 7 | 6 | 9 | 6 | 10 | 7 | 10 |
| A6.100 | 250 | 10 | 10 | 6 | 10 | 10 | 10 | 10 | 10 |
| A8.008 | 250 | 10 | 10 | 0 | 0 | 10 | 10 | nt | 10 |
| A8.080 | 250 | 9 | 10 | 0 | 8 | 9 | 10 | 0 | 10 |
| B1.008 | 250 | 10 | 9 | 9 | 10 | 9 | 10 | 10 | 10 |
| B1.080 | 250 | 10 | 10 | 9 | 9 | 0 | 8 | 0 | 10 |
| B1.096 | 250 | 7 | nt | 7 | 7 | 7 | 10 | 10 | 10 |
| B1.170 | 250 | 9 | 9 | 8 | 9 | 9 | 9 | 9 | 10 |

### EXAMPLE B2

### Post-Emergence Herbicidal Action

In a greenhouse, monocotyledonous and dicotyledonous test plants are grown in standard soil in plastic pots and at the 4- to 6-leaf stage are sprayed with an aqueous suspension of the test compounds of formula I prepared from a 25% wettable powder (Example F3, b) according to WO 97/34485) or with an emulsion of the test compounds of formula I prepared from a 25% emulsifiable concentrate (Example F1, c) according to WO 97/34485), in a concentration corresponding to 125 g or 250 g of active ingredient/ha (500 litres of water/ha). The test plants are then grown on in a greenhouse under optimum conditions. After a test duration of about 18 days, the test is evaluated in accordance with a scale of nine ratings (10=total damage, 0=no action). Ratings of from 10 to 7 (especially from 10 to 8) indicate good to very good herbicidal action. The compounds of formula I exhibit a strong herbicidal action in this test.

TABLE B2
Post-emergence action of compounds of formula I:

| Ex. No | gr. a.i./ha | Echinochloa | Euphorbia | Xanthium | Ipomea | Amaranthus | Chenopodium | Sinapis | Stellaria |
|---|---|---|---|---|---|---|---|---|---|
| A1.001 | 125 | 4 | 4 | 8 | 8 | 8 | 9 | 8 | 8 |
| A1.007 | 250 | 8 | 4 | 9 | 9 | 9 | 10 | 8 | 7 |
| A1.019 | 250 | 8 | 9 | 9 | 9 | 9 | 9 | 8 | 8 |
| A1.031 | 250 | 7 | 8 | 9 | 9 | 9 | 10 | 8 | 9 |
| A1.037 | 250 | 4 | 8 | 9 | 9 | 9 | 9 | 8 | 8 |
| A1.043 | 250 | 7 | 7 | 9 | 9 | 9 | 9 | 6 | 9 |
| A1.049 | 250 | 8 | 9 | 9 | 9 | 9 | 8 | 8 | 8 |
| A1.073 | 250 | 9 | 9 | 9 | 10 | 10 | 10 | 10 | 10 |
| A1.079 | 250 | 7 | 8 | 7 | 8 | 9 | 9 | 9 | 9 |
| A1.091 | 250 | 9 | 8 | 9 | 9 | 9 | 10 | 8 | 10 |
| A1.109 | 250 | 8 | 10 | 9 | 9 | 9 | 10 | 3 | 5 |
| A1.115 | 250 | 7 | 8 | 9 | 7 | 9 | 9 | 3 | 9 |
| A1.181 | 250 | 4 | 8 | 8 | 8 | 9 | 8 | 5 | 7 |
| A1.202 | 250 | 8 | 9 | 9 | 9 | 9 | 8 | 8 | 7 |
| A6.073 | 250 | 9 | 9 | 9 | 10 | 10 | 10 | 10 | 9 |
| A6.082 | 250 | 7 | 7 | 7 | 8 | 8 | 9 | 5 | 9 |
| A6.091 | 250 | 9 | 8 | 9 | 8 | 8 | 9 | 8 | 9 |
| A6.097 | 250 | 7 | 7 | 7 | 7 | 7 | 9 | 8 | 9 |
| A6.100 | 250 | 7 | 7 | 7 | 9 | 9 | 10 | 8 | 9 |
| A7.008 | 250 | 7 | 7 | 8 | 7 | 5 | 9 | 9 | 9 |
| A7.009 | 250 | 7 | 7 | 7 | 7 | 4 | 9 | 8 | 7 |
| A8.008 | 250 | 8 | 8 | 9 | 9 | 9 | 8 | 7 | 6 |
| A8.062 | 250 | 9 | 9 | 0 | 8 | 9 | 10 | 9 | 5 |
| A8.080 | 250 | 9 | 9 | 8 | 10 | 9 | 10 | 10 | 10 |
| A8.095 | 250 | 9 | 0 | 8 | 9 | 9 | 5 | 8 | 7 |
| A8.174 | 250 | 0 | 7 | 7 | 8 | 8 | 9 | 7 | 7 |
| B1.004 | 250 | 8 | 9 | 9 | 8 | 8 | 9 | 10 | 8 |
| B1.005 | 250 | 4 | 9 | 6 | 8 | 9 | 9 | 9 | 8 |
| B1.008 | 250 | 9 | 8 | nt | 9 | 9 | 10 | 7 | 8 |
| B1.039 | 250 | 9 | 9 | 8 | 8 | 6 | 8 | 9 | 9 |
| B1.050 | 250 | 4 | 9 | 8 | 7 | 9 | 9 | 9 | 7 |
| B1.056 | 250 | 9 | 9 | 0 | 10 | 9 | 8 | 8 | 7 |
| B1.062 | 250 | 4 | 9 | 6 | 7 | 9 | 9 | 8 | 8 |
| B1.080 | 250 | 9 | 10 | 8 | 10 | 10 | 10 | 10 | 10 |
| B1.096 | 250 | 6 | 7 | 8 | 7 | 7 | 10 | 9 | 8 |
| B1.158 | 250 | 4 | 7 | 5 | 8 | 7 | 8 | 7 | 7 |
| B1.170 | 250 | 9 | 7 | 6 | 0 | 9 | 9 | 9 | 9 |
| B1.194 | 250 | 9 | 9 | 9 | 7 | 9 | 9 | 7 | 8 |

In a different test arrangement, the Examples according to Table B3 likewise exhibit good to very good post-emergence action on selected test plants.

TABLE B3

| Ex. No | gr. a.i./ha | Amaranthus | Solanum | Nasturtium | Stellaria |
|---|---|---|---|---|---|
| A1.025 | 250 | 9 | 9 | 9 | 9 |
| A1.097 | 250 | 9 | 9 | 10 | 9 |
| A1.175 | 250 | 7 | 9 | 8 | 7 |
| A1.209 | 250 | 7 | 9 | 9 | 7 |
| A1.211 | 250 | 9 | 9 | 10 | 7 |
| A1.213 | 250 | 9 | 9 | 9 | 9 |
| A1.219 | 250 | 9 | 9 | 10 | 10 |
| A1.220 | 250 | 9 | 9 | 10 | 10 |
| A1.221 | 250 | 9 | 9 | 10 | 9 |
| A1.222 | 250 | 9 | 9 | 10 | 9 |
| A1.223 | 250 | 8 | 9 | 10 | 9 |
| A1.237 | 250 | 9 | 10 | 9 | 7 |
| B1.211 | 250 | 9 | 9 | 10 | 8 |
| B1.223 | 250 | 8 | 9 | 10 | 10 |
| B1.225 | 250 | 8 | 9 | 10 | 10 |
| B1.226 | 250 | 8 | 9 | 10 | 9 |
| B1.238 | 250 | 9 | 9 | 8 | 7 |
| B1.297 | 250 | 9 | 9 | 10 | 9 |

1. A compound of formula I 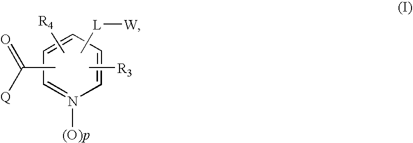
wherein
L is either a direct bond, an —O—, —S—, —S(O)—, —SO₂—, —N(R₅ₐ)—, —SO₂N(R_{5b})—, —N(R_{5b})SO₂—, —C(O)N(R_{5c})- or —N(R_{5c})C(O)— bridge, or a C₁-C₄alkylene, C₂-C₄alkenylene or C₂-C₄alkynylene chain which may be mono- or poly-substituted by R₅ and/or interrupted once or twice by an —O—, —S—, —S(O)—, —SO₂—, N(R_{5d})—, —SO₂N(R₅ₑ)—, —N(R₅ₑ)SO₂—, —C(O)N(R_{5f})— and/or —N(R_{5f})C(O)— bridge, and when two such bridges are present those bridges are separated at least by one carbon atom, and W is bonded to L by way of a carbon atom or a —N(R₅ₑ)SO₂— or —N(R_{5f})C(O)— bridge when the bridge L is bonded to the nitrogen atom of W;
W is a 4- to 7-membered, saturated, partially saturated or unsaturated ring system U 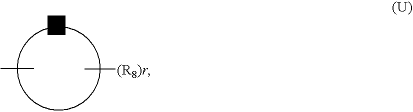
which contains a ring element U₁, and may contain from one to four further ring nitrogen atoms, and/or two further ring oxygen atoms, and/or two further ring sulfur atoms and/or one or two further ring elements U₂, and the ring system U may be mono- or poly-substituted at a saturated or unsaturated ring carbon atom and/or at a ring nitrogen atom by a group R₈, and two substituents R₈ together are a further fused-on or spirocyclic 3- to 7-membered ring system which may be unsaturated, partially saturated or fully saturated and may in turn be substituted by one or more groups R₈ₐ and/or interrupted once or twice by a ring element —O—, —S—, —N(R_{8b})— and/or —C(═O)—; and U₁ and U₂ are each independently of the other(s) —C(═O)—, —C(═S)—, —C(═NR₆)—, —(N═O)—, —S(═O)— or —SO₂—;
R₃ and R₄ are each independently of the other C₁-C₃alkyl, C₁-C₃haloalkyl, C₁-C₃alkoxy-C₁-C₃alkyl, hydrogen, hydroxy, mercapto, halogen, C₁-C₃alkoxy, C₁-C₃haloalkoxy, C₁-C₃alkoxy-C₁-C₃alkoxy, C₁-C₃alkylthio, C₁-C₃alkylsulfinyl, C₁-C₃alkylsulfonyl, C₁-C₃haloalkylthio, C₁-C₃haloalkylsulfinyl, C₁-C₃haloalkylsulfonyl or C₁-C₃alkylsulfonyloxy;
R₅ is halogen, C₁-C₃alkyl, C₁-C₃alkoxy, C₁-C₃alkylthio, C₁-C₃alkylsulfinyl, C₁-C₃alkylsulfonyl, C₁-C₃alkoxy-C₁-C₃alkyl or C₁-C₃alkoxy-C₁-C₃alkoxy;
R₅ₐ, R_{5b} and R₅ₑ are independently hydrogen, C₁-C₆alkyl, C₃-C₆alkenyl, C₃-C₆alkynyl or C₁-C₃alkoxy-C₁-C₃alkyl;
R_{5d} is hydrogen, C₁-C₆alkyl, C₃-C₆alkenyl, C₃-C₆alkynyl, C₁-C₃alkoxy-C₁-C₃alkyl, benzyl, cyano, formyl, C₁-C₄alkylcarbonyl, C₁-C₄alkoxycarbonyl, C₁-C₄alkylsulfonyl or phenylsulfonyl, it being possible for the phenyl-containing groups to be substituted by R₇;
R_{5c} and R_{5f} are each independently of the other hydrogen or C₁-C₃alkyl;
R₆ is C₁-C₆alkyl, hydroxy, C₁-C₆alkoxy, cyano or nitro;
R₇ is halogen, C₁-C₃alkyl, C₁-C₃haloalkyl, hydroxy, C₁-C₃alkoxy, C₁-C₃haloalkoxy, cyano or nitro;
each R₈ independently is hydrogen, halogen, C₁-C₆alkyl, C₁-C₆haloalkyl, C₃-C₆cycloalkyl, C₂-C₆alkenyl, C₂-C₆alkynyl, hydroxy, C₁-C₆alkoxy, C₁-C₆haloalkoxy, C₃-C₆alkenyloxy, C₃-C₆alkynyloxy, C₁-C₃alkoxy-C₁-C₃alkoxy, mercapto, C₁-C₆alkylthio, C₁-C₆alkylsulfinyl, C₁-C₆alkylsulfonyl, C₁-C₆-alkylsulfonyloxy, C₁-C₆haloalkylsulfonyloxy, C₃-C₆alkenylthio, C₃-C₆alkynylthio, amino, C₁-C₆alkylamino, di(C₁-C₆alkyl)amino, C₁-C₃alkoxy-C₁-C₃alkyl, formyl, C₁-C₄alkylcarbonyl, C₁-C₄alkoxycarbonyl, benzyloxycarbonyl, C₁-C₄alkylthiocarbonyl, carboxy, cyano, carbamoyl, phenyl, benzyl, heteroaryl or heterocyclyl, it being possible for the phenyl, benzyl, heteroaryl and heterocyclyl groups to be mono- or poly-substituted by R₇ₐ;
each R₇ₐ independently is halogen, C₁-C₃alkyl, C₁-C₃haloalkyl, hydroxy, C₁-C₃alkoxy, C₁-C₃haloalkoxy, cyano or nitro;
each R₈ₐ independently is halogen, C₁-C₆alkyl, C₁-C₆haloalkyl, C₃-C₆cycloalkyl, C₂-C₆alkenyl, C₂-C₆alkynyl, hydroxy, C₁-C₆alkoxy, C₁-C₆haloalkoxy, C₃-C₆alkenyloxy, C₃-C₆alkynyloxy, mercapto, C₁-C₆alkylthio, C₁-C₆alkylsulfinyl, C₁-C₆alkylsulfonyl, C₁-C₄alkylcarbonyl, C₁-C₄alkoxycarbonyl, cyano or nitro;
R_{8b} is hydrogen, C₁-C₃alkyl, C₃-C₆alkenyl, C₃-C₆alkynyl, C₁-C₃alkoxy-C₁-C₃alkyl or benzyl, it being possible for the phenyl group to be substituted by R_{7b};
R_{7b} is halogen, C₁-C₃alkyl, C₁-C₃haloalkyl, hydroxy, C₁-C₃alkoxy, C₁-C₃haloalkoxy, cyano or nitro;
p is 0 or 1;
r is 1,2,3,4, 5 or 6;
with the provisos that
a) R₈ and R₈ₐ as halogen or hydrogenmercapto cannot be bonded to a nitrogen atom,
b) U₁ as —C(═O)— or —C(═S)— does not form a tautomeric form with a substituent Rₐ as hydrogen when the radical W is bonded to the pyridyl group by way of a C₁-C₄alkylene, C₂-C₄alkenylene or C₂-C₄alkynylene chain L that is interrupted by —O—, —S—, —S(O)—, —SO₂—, —N(R_{5d})—, —SO₂N(R₅ₑ)— or —N(R₅ₑ)SO₂—,
c) U₁ as —C(═S)— does not form a tautomeric form with a substituent Rₐ as hydrogen when the radical W is bonded to the pyridyl group by way of a —CH═CH— or —C≡C— bridge L or by way of a C₁-C₄alkylene chain L that is interrupted by —O—, —S—, —S(O)—, —SO₂— or —N(C₁-C₄alkyl)—,
d) U₁ as —C(═S)— or —C(═NR₆)— wherein R₆ is C₁-C₆alkyl or C₁-C₆alkoxy does not form a tautomeric form with a substituent Rₐ as hydrogen when the radical W is bonded to the pyridyl group directly or by way of a C₁-C₄alkylene chain L;
either
Q is a group Q₁ 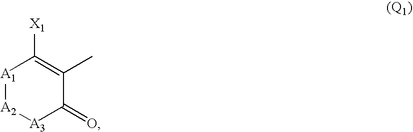
wherein
A₁ is C(R₁₁R₁₂) or NR₁₃;
A₂ is C(R₁₄R₁₅)ₘ, C(O), oxygen, NR₁₆ or S(O)_{q};
A₃ is C(R₁₇R₁₈) or NR₁₉;
with the proviso that A₂ is other than S(O)_{q} when A₁ is NR₁₃ and/or A₃ is NR₁₉;
X₁ is hydroxy, O⁻M⁺, wherein M⁺ is a metal cation or an ammonium cation; halogen or S(O)ₙR₉,
wherein
m is 1 or 2;
q, n and k are each independently of the others 0, 1 or 2;
R₉ is C₁-C₁₂alkyl, C₂-C₁₂alkenyl, C₂-C₁₂alkynyl, C₃-C₁₂allenyl, C₃-C₁₂cycloalkyl, C₅-C₁₂cycloalkenyl, R₁₀—C₁-C₁₂alkylene or R₁₀—C₂-C₁₂alkenylene, wherein the alkylene or alkenylene chain may be interrupted by —O—, —S(O)ₖ— and/or —C(O)— and/or mono- to penta-substituted by R₂₀; or phenyl, which may be mono- to penta-substituted by R_{7c};
R_{7c} is halogen, C₁-C₃alkyl, C₁-C₃haloalkyl, hydroxy, C₁-C₃alkoxy, C₁-C₃haloalkoxy, cyano or nitro;
R₁₀ is halogen, cyano, rhodano, hydroxy, C₁-C₆alkoxy, C₂-C₆alkenyloxy, C₂-C₆alkynyloxy, C₁-C₆alkylthio, C₁-C₆alkylsulfinyl, C₁-C₆alkylsulfonyl, C₂-C₆alkenylthio, C₂-C₆alkynylthio, C₁-C₆alkylsulfonyloxy, phenylsulfonyloxy, C₁-C₆alkylcarbonyloxy, benzoyloxy, C₁-C₄alkoxy-carbonyloxy, C₁-C₆alkylcarbonyl, C₁-C₄alkoxycarbonyl, benzoyl, aminocarbonyl, C₁-C₄alkyl-aminocarbonyl, C₃-C₆cycloalkyl, phenyl, phenoxy, phenylthio, phenylsulfinyl or phenylsulfonyl; it being possible for the phenyl-containing groups in turn to be substituted by R_{7d};
R_{7d} is halogen, C₁-C₃alkyl, C₁-C₃haloalkyl, hydroxy, C₁-C₃alkoxy, C₁-C₃haloalkoxy, cyano or nitro;
R₂₀ is hydroxy, halogen, C₁-C₆alkyl, C₁-C₆alkoxy, C₁-C₆alkylthio, C₁-C₆alkylsulfinyl, C₁-C₆alkylsulfonyl, cyano, carbamoyl, carboxy, C₁-C₄alkoxycarbonyl or phenyl; it being possible for phenyl to be substituted by R₇ₑ;
R₇ₑ is halogen, C₁-C₃alkyl, C₁-C₃haloalkyl, hydroxy, C₁-C₃alkoxy, C₁-C₃haloalkoxy, cyano or nitro;
R₁₁ and R₁₇ are each independently of the other hydrogen, C₁-C₄alkyl, C₂-C₄alkenyl, C₂-C₄alkynyl, C₁-C₄alkylthio, C₁-C₄alkylsulfinyl, C₁-C₄alkylsulfonyl, C₁-C₄alkoxycarbonyl, hydroxy, C₁-C₄alkoxy, C₃-C₄alkenyloxy, C₃-C₄alkynyloxy, hydroxy-C₁-C₄alkyl, C₁-C₄alkylsulfonyloxy-C₁-C₄alkyl, halogen, cyano or nitro;
or, when A₂ is C(R₁₄R₁₅)ₘ, R₁₇ together with R₁₁ forms a direct bond or a C₁-C₃alkylene bridge;
R₁₂ and R₁₈ are each independently of the other hydrogen, C₁-C₄alkyl or C₁-C₄alkylthio, C₁-C₄alkylsulfinyl or C₁-C₄alkylsulfonyl;
or R₁₂ together with R₁₁, and/or R₁₈ together with R₁₇ form a C₂-C₅alkylene chain which may be interrupted by —O—, —C(O)—, —O— and —C(O)— or —S(O)ₜ—;
R₁₃ and R_{1g} are each independently of the other hydrogen, C₁-C₄alkyl, C₁-C₄haloalkyl, C₃-C₄alkenyl, C₃-C₄alkynyl or C₁-C₄alkoxy;
R₁₄ is hydrogen, hydroxy, C₁-C₄alkyl, C₁-C₄haloalkyl, C₁-C₃hydroxyalkyl, C₁-C₄alkoxy-C₁-C₃-alkyl, C₁-C₄alkylthio-C₁-C₃alkyl, C₁-C₄alkylcarbonyloxy-C₁-C₃alkyl, C₁-C₄alkylsulfonyloxy-C₁-C₃alkyl, tosyloxy-C₁-C₃alkyl, di(C₁-C₄alkoxy)-C₁-C₃alkyl, C₁-C₄alkoxycarbonyl, C₃-C₅-oxacycloalkyl, C₃-C₅thiacycloalkyl, C₃-C₄dioxacycloalkyl, C₃-C₄dithiacycloalkyl, C₃-C₄oxathiacycloalkyl, formyl, C₁-C₄alkoxyiminomethyl, carbamoyl, C₁-C₄alkylaminocarbonyl or di-(C₁-C₄alkyl)aminocarbonyl;
or R₁₄ together with R₁₁, R₁₂, R₁₃, R₁₅, R₁₇, R₁₈ or R₁₉ or, when m is 2, also together with R₁₄ forms a direct bond or a C₁-C₄alkylene bridge;
R₁₅ is hydrogen, C₁-C₃alkyl or C₁-C₃haloalkyl;
R₁₆ is hydrogen, C₁-C₃alkyl, C₁-C₃haloalkyl, C₁-C₄alkoxycarbonyl, C₁-C₄alkylcarbonyl or N,N-di(C₁-C₄alkyl)aminocarbonyl; or
Q is a group Q₂ 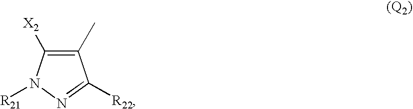
wherein
R₂₁ and R₂ are hydrogen or C₁-C₄alkyl;
X₂ is hydroxy, O⁻M⁺, wherein M⁺ is an alkali metal cation or ammonium cation; halogen, C₁-C₁₂alkylsulfonyloxy, C₁-C₁₂alkylthio, C₁-C₁₂alkylsulfinyl, C₁-C₁₂alkylsulfonyl, C₁-C₁₂haloalkylthio, C₁-C₁₂haloalkylsulfinyl, C₁-C₁₂haloalkylsulfonyl, C₁-C₆alkoxy-C₁-C₆alkylthio, C₁-C₆alkoxy-C₁-C₆alkylsulfinyl, C₁-C₆alkoxy-C₁-C₆alkylsulfonyl, C₃-C₁₂alkenylthio, C₃-C₁₂alkenylsulfinyl, C₃-C₁₂alkenylsulfonyl, C₃-C₁₂alkynylthio, C₃-C₁₂alkynylsulfinyl, C₃-C₁₂alkynylsulfonyl, C₁-C₄alkoxycarbonyl-C₁-C₄alkylthio, C₁-C₄alkoxycarbonyl-C₁-C₄alkylsulfinyl, C₁-C₄alkoxycarbonyl-C₁-C₄alkylsulfonyl, benzyloxy or phenylcarbonylmethoxy; it being possible for the phenyl-containing groups to be substituted by R_{7f};
R_{7f} is halogen, C₁-C₃alkyl, C₁-C₃haloalkyl, hydroxy, C₁-C₃alkoxy, C₁-C₃haloalkoxy, cyano or nitro; or
Q is a group Q₃ 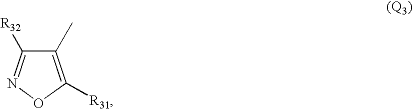
wherein
R₃₁ is C₁-C₆alkyl, C₁-C₆haloalkyl, C₃-C₆cycloalkyl or halo-substituted C₃-C₆cycloalkyl;
R₃₂ is hydrogen, C₁-C₄alkoxycarbonyl, carboxy or a group S(O)_{S}R₃₃;
R₃₃ is C₁-C₆alkyl or C₁-C₃alkylene, which may be substituted by halogen, C₁-C₃alkoxy, C₂-C₃alkenyl or by C₂-C₃alkynyl; and
s is 0, 1 or 2; or
Q is a group Q₄ 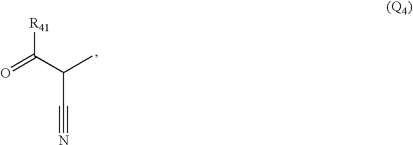
wherein
R₄, is C₁-C₆alkyl, C₁-C₆haloalkyl, C₃-C₆cycloalkyl or halo-substituted C₃-C₆cycloalkyl;
or an agrochemically acceptable salt or any stereoisomer or tautomer of a compound of formula I.

2. A compound of formula II 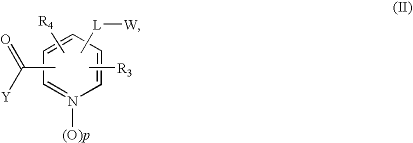
wherein Y is chlorine, cyano, hydroxy, C₁-C₄alkoxy, benzyloxy, phenoxy, allyloxy, a group 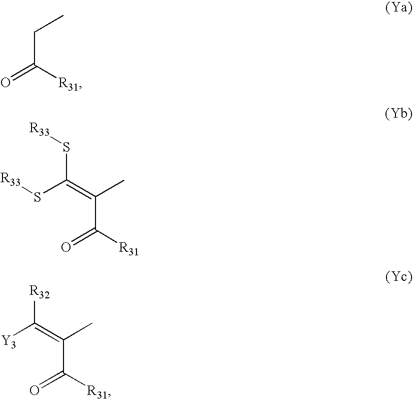
or a group Q₀, wherein Q₀ is accordingly a group Q linked to oxygen and Q, L, U₁, R₁, R₂, R₃, R₄, R₃₁, R₃₂, R₃₃ and p are as defined for formula I in claim 1.

3. A herbicidal and plant-growth-inhibiting composition, which comprises a herbicidally effective amount of a compound of formula I, according to claim 1, on an inert carrier.

4. A method of controlling undesired plant growth, which comprises applying a herbicidally effective amount of a compound of formula I, according to claim 1, or of a composition comprising such a compound, to the plants or to the locus thereof.

5. A method of inhibiting plant growth, which comprises applying a herbicidally effective amount of a compound of formula I, according to claim 1, or of a composition comprising such a compound, to the plants or to the locus thereof.
